(19) United States
(12) Patent Application Publication
(10) Pub. No.: US 2018/0305775 A1
(43) Pub. Date: Oct. 25, 2018

(54) METHODS FOR PREDICTING PALM OIL YIELD OF A TEST OIL PALM PLANT

(71) Applicant: SIME DARBY PLANTATION INTELLECTUAL PROPERTY SDN. BHD., Petaling Jaya (MY)

(72) Inventors: Qi Bin Kwong, Serdang (MY); Ai Ling Ong, Serdang (MY); Chee Keng Teh, Banting (MY); Mohaimi Mohamed, Banting (MY); Fook Tim Chew, National University of Singapore (SG); David Rose Appleton, Serdang (MY); Harikrishna Kulaveerasingam, Carey Island (MY)

(21) Appl. No.: 15/767,597

(22) PCT Filed: Oct. 21, 2016
(86) PCT No.: PCT/MY2016/000072

§ 371 Date: Apr. 11, 2018

(30) Foreign Application Priority Data
Oct. 23, 2015 (MY) PI 2015703819

(51) Int. Cl. C12Q 1/6895; G06F 19/18

(52) U.S. Cl. CPC C12Q 1/6895; C12Q 2600/13; C12Q 2600/156; G06F 19/18

(57) ABSTRACT

Methods for predicting palm oil yield of a test oil palm plant are disclosed. The methods comprise determining, from a sample of a test oil palm plant of a population, at least a first SNP genotype, corresponding to a first SNP marker, located in a first QTL for a high-oil-production trait and associated, after stratification and kinship correction, with the high-oil-production trait with a genome-wide −log₁₀(p-value) of at least 3.0 in the population or having a linkage disequilibrium r² value of at least 0.2 with respect to a first other SNP marker linked thereto and associated, after stratification and kinship correction, with the high-oil-production trait with a genome-wide −log₁₀(p-value) of at least 3.0 in the population. The methods also comprise comparing the first SNP genotype to a corresponding first reference SNP genotype and predicting palm oil yield of the test plant based on extent of matching of the SNP genotypes.

### TECHNICAL FIELD

This application relates to methods for predicting palm oil yield of a test oil palm plant, and more particularly to methods for predicting palm oil yield of a test oil palm plant comprising determining, from a sample of a test oil palm plant of a population of oil palm plants, at least a first single nucleotide polymorphism (SNP) genotype of the test oil palm plant, the first SNP genotype corresponding to a first SNP marker, comparing the first SNP genotype of the test oil palm plant to a corresponding first reference SNP genotype indicative of the high-oil-production trait in the same genetic background as the population, and predicting palm oil yield of the test oil palm plant based on the extent to which the first SNP genotype of the test oil palm plant matches the corresponding first reference SNP genotype.

### BACKGROUND ART

The African oil palm Elaeis guineensis is an important oil-food crop. Oil palm plants are monoecious, i.e. single plants produce both male and female flowers, and are characterized by alternating series of male and female inflorescences. The male inflorescence is made up of numerous spikelets, and can bear well over 100,000 flowers. Oil palm is naturally cross-pollinated by insects and wind. The female inflorescence is a spadix which contains several thousands of flowers borne on thorny spikelets. A bunch carries 500 to 4,000 fruits. The oil palm fruit is a sessile drupe that is spherical to ovoid or elongated in shape and is composed of an exocarp, a mesocarp containing palm oil, and an endocarp surrounding a kernel.

Oil palm is important both because of its high yield and because of the high quality of its oil. Regarding yield, oil palm is the highest yielding oil-food crop, with a recent average yield of 3.67 tonnes per hectare per year and with best progenies known to produce about 10 tonnes per hectare per year. Oil palm is also the most efficient plant known for harnessing the energy of sunlight for producing oil. Regarding quality, oil palm is cultivated for both palm oil, which is produced in the mesocarp, and palm kernel oil, which is produced in the kernel. Palm oil in particular is a balanced oil, having almost equal proportions of saturated fatty acids (≈55% including 45% of palmitic acid) and unsaturated fatty acids (≈45%), and it includes beta carotene. The palm kernel oil is more saturated than the mesocarp oil. Both are low in free fatty acids. The current combined output of palm oil and palm kernel oil is about 50 million tonnes per year, and demand is expected to increase substantially in the future with increasing global population and per capita consumption of oils and fats.

Although oil palm is the highest yielding oil-food crop, current oil palm crops produce well below their theoretical maximum, suggesting potential for improving yields of palm oil through improved selection and identification of high yielding oil palm plants. Conventional methods for identifying potential high-yielding palms, for use in crosses to generate progeny with higher yields as well as for commercial production of palm oil, require cultivation of palms and measurement of production of oil thereby over the course of many years, though, which is both time and labor intensive. Moreover, the conventional methods are based on direct measurement of oil content of sampled fruits, and thus result in destruction of the sampled fruits. In addition, conventional breeding techniques for propagation of oil palm for oil production are also time and labor intensive, particularly because the most productive, and thus commercially relevant, palms exhibit a hybrid phenotype which makes propagation thereof by direct hybrid crosses impractical. Quantitative trait loci (also termed QTL) marker programs based on linkage analysis have been implemented in oil palm with the aim of improving upon conventional breeding techniques, as taught for example by Billotte et al., Theoretical & Applied Genetics 120:1673-1687 (2010). Linkage analysis is based on recombination observed in a family within recent generations and often identifies poorly localized QTLs for complex phenotypes, though, and thus large families are needed for better detection and confirmation of QTLs, limiting practicality of this approach for oil palm. QTL marker programs based on association analysis for the purpose of identifying candidate genes may be a possibility for oil palm too, as discussed for example by Ong et al., WO2014/129885, with respect to plant height. A focus on identifying candidate genes may be of limited benefit in the context of traits that are determined by multiple genes though, particularly genes that exhibit low penetrance with respect to the trait. QTL marker programs based on genome-wide association studies have been carried out in human and rice, among others, as taught by Hirota et al., Nature Genetics 44:1222-1226 (2012), and Huang et al., Nature Genetics 42:961-967 (2010), respectively. Application of this approach to oil palm has not been practical, though, because commercial palms tend to be generated from genetically narrow breeding materials. Accordingly, a need exists to improve oil palm through improved methods for predicting palm oil yields of oil palm plants.

### DISCLOSURE OF INVENTION

In one example embodiment, a method for predicting palm oil yield of a test oil palm plant is disclosed. The method comprises a step of (i) determining, from a sample of a test oil palm plant of a population of oil palm plants, at least a first single nucleotide polymorphism (SNP) genotype of the test oil palm plant. The first SNP genotype corresponds to a first SNP marker. The first SNP marker is located in a first quantitative trait locus (QTL) for a high-oil-production trait. The first SNP marker also is associated, after stratification and kinship correction, with the high-oil-production trait with a genome-wide −log₁₀(p-value) of at least 3.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 3.0 in the population. The method also comprises a step of (ii) comparing the first SNP genotype of the test oil palm plant to a corresponding first reference SNP genotype indicative of the high-oil-production trait in the same genetic background as the population. The method also comprises a step of (iii) predicting palm oil yield of the test oil palm plant based on the extent to which the first SNP genotype of the test oil palm plant matches the corresponding first reference SNP genotype. The first QTL is a region of the oil palm genome corresponding to one of:

(1) QTL region 1, extending from nucleotide 18204491 to 18358401 of chromosome 1;
(2) QTL region 2, extending from nucleotide 18922390 to 19167923 of chromosome 1;
(3) QTL region 3, extending from nucleotide 19188077 to 19685080 of chromosome 1;
(4) QTL region 4, extending from nucleotide 23276098 to 23456770 of chromosome 1;
(5) QTL region 5, extending from nucleotide 26021716 to 26066534 of chromosome 1;
(6) QTL region 6, extending from nucleotide 28110016 to 28234799 of chromosome 1;
(7) QTL region 7, extending from nucleotide 29798161 to 30164329 of chromosome 1;
(8) QTL region 8, extending from nucleotide 30684639 to 31160129 of chromosome 1;
(9) QTL region 9, extending from nucleotide 37811723 to 38637229 of chromosome 1;
(10) QTL region 10, extending from nucleotide 38659012 to 39206652 of chromosome 1;
(11) QTL region 11, extending from nucleotide 39243858 to 39842157 of chromosome 1;
(12) QTL region 12, extending from nucleotide 61305818 to 61572106 of chromosome 1;
(13) QTL region 13, extending from nucleotide 1068379 to 1516571 of chromosome 2;
(14) QTL region 14, extending from nucleotide 1616491 to 2016169 of chromosome 2;
(15) QTL region 15, extending from nucleotide 17637996 to 17959911 of chromosome 2;
(16) QTL region 16, extending from nucleotide 20732085 to 20977490 of chromosome 2;
(17) QTL region 17, extending from nucleotide 31844836 to 31980071 of chromosome 2;
(18) QTL region 18, extending from nucleotide 50449700 to 50857310 of chromosome 2;
(19) QTL region 19, extending from nucleotide 50879601 to 51539414 of chromosome 2;
(20) QTL region 20, extending from nucleotide 52821582 to 52960520 of chromosome 2;
(21) QTL region 21, extending from nucleotide 42585292 to 42728875 of chromosome 3;
(22) QTL region 22, extending from nucleotide 9561644 to 9701199 of chromosome 4;
(23) QTL region 23, extending from nucleotide 12469969 to 13409114 of chromosome 4;
(24) QTL region 24, extending from nucleotide 14672228 to 14789226 of chromosome 4;
(25) QTL region 25, extending from nucleotide 395189 to 842107 of chromosome 5;
(26) QTL region 26, extending from nucleotide 47205529 to 47293291 of chromosome 5;
(27) QTL region 27, extending from nucleotide 48857594 to 48932286 of chromosome 5;
(28) QTL region 28, extending from nucleotide 5943980 to 6002717 of chromosome 6;
(29) QTL region 29, extending from nucleotide 6337822 to 6563232 of chromosome 6;
(30) QTL region 30, extending from nucleotide 6818733 to 7281658 of chromosome 6;
(31) QTL region 31, extending from nucleotide 17578027 to 18209857 of chromosome 6;
(32) QTL region 32, extending from nucleotide 26204516 to 26755007 of chromosome 6;
(33) QTL region 33, extending from nucleotide 36492757 to 36494757 of chromosome 6;
(34) QTL region 34, extending from nucleotide 219790 to 1533149 of chromosome 7;
(35) QTL region 35, extending from nucleotide 8700733 to 9242332 of chromosome 8;
(36) QTL region 36, extending from nucleotide 23767318 to 23957652 of chromosome 8;
(37) QTL region 37, extending from nucleotide 26648547 to 26848102 of chromosome 8;
(38) QTL region 38, extending from nucleotide 606020 to 1309231 of chromosome 9;
(39) QTL region 39, extending from nucleotide 3499347 to 3638435 of chromosome 9;
(40) QTL region 40, extending from nucleotide 28437588 to 28513671 of chromosome 9;
(41) QTL region 41, extending from nucleotide 28581068 to 28912034 of chromosome 9;
(42) QTL region 42, extending from nucleotide 32327318 to 32434321 of chromosome 9;
(43) QTL region 43, extending from nucleotide 32538074 to 32540074 of chromosome 9;
(44) QTL region 44, extending from nucleotide 32775289 to 33054696 of chromosome 9;
(45) QTL region 45, extending from nucleotide 33133902 to 33254107 of chromosome 9;
(46) QTL region 46, extending from nucleotide 15342814 to 15405953 of chromosome 10;
(47) QTL region 47, extending from nucleotide 15933273 to 15943963 of chromosome 11;
(48) QTL region 48, extending from nucleotide 12178551 to 12249693 of chromosome 12;
(49) QTL region 49, extending from nucleotide 2052746 to 2447722 of chromosome 13;
(50) QTL region 50, extending from nucleotide 14345084 to 14709650 of chromosome 13;
(51) QTL region 51, extending from nucleotide 22031000 to 22147560 of chromosome 13;
(52) QTL region 52, extending from nucleotide 23588504 to 24307350 of chromosome 15;
(53) QTL region 53, extending from nucleotide 1511530 to 1596020 of chromosome 16;
(54) QTL region 54, extending from nucleotide 2684531 to 2803682 of chromosome 16;
(55) QTL region 55, extending from nucleotide 5535711 to 5995857 of chromosome 16;
(56) QTL region 56, extending from nucleotide 8379248 to 8554851 of chromosome 16; or
(57) QTL region 57, extending from nucleotide 8883687 to 9269845 of chromosome 16.

In another example embodiment, a method for predicting palm oil yield of a test oil palm plant is disclosed. The method comprises a step of (i) determining, from a sample of a test oil palm plant of a population of oil palm plants, at least a first single nucleotide polymorphism (SNP) genotype to a tenth SNP genotype of the test oil palm plant. The first SNP genotype to the tenth SNP genotype correspond to a first SNP marker to a tenth SNP marker, respectively. The first SNP marker to the tenth SNP marker are located in a first quantitative trait locus (QTL) to a tenth QTL, respectively, for a high-oil-production trait. The first SNP marker to the tenth SNP marker also are associated, after stratification and kinship correction, with the high-oil-production trait with a genome-wide −log₁₀(p-value) of at least 3.0 in the population or have a linkage disequilibrium r² value of at least 0.2 with respect to a first other SNP marker to a tenth other SNP marker, respectively, that are linked thereto and associated, after stratification and kinship correction, with the high-oil-production trait with a genome-wide −log₁₀(p-value) of at least 3.0 in the population. The method also comprises a step of (ii) comparing the first SNP genotype to the tenth SNP genotype of the test oil palm plant to a corresponding first reference SNP genotype to a corresponding tenth reference SNP genotype, respectively, indicative of the high-oil-production trait in the same genetic background as the population. The method also comprises a step of (iii) predicting palm oil yield of the test oil palm plant based on the extent to which the first SNP genotype to the tenth SNP genotype of the test oil palm plant matches the corresponding first reference SNP genotype to the corresponding tenth reference SNP genotype, respectively.

### BRIEF DESCRIPTION OF DRAWINGS

FIG. 1 shows (A, B) quantile-quantile Q-Q plots of observed −log₁₀(p-values) versus expected −log₁₀(p-values) for genome-wide association study (also termed GWAS), based on a compressed mixed linear model (also termed MLM), in (A) an Ulu Remis dura×AVROS pisifera population and (B) a Banting dura×AVROS pisifera population.

FIG. 2 shows (A, B) Manhattan plots, based on a compressed mixed linear model (also termed MLM), in (A) an Ulu Remis dura×AVROS pisifera population and (B) a Banting dura×AVROS pisifera population.

FIG. 3 is an illustration of an approach for defining a range of a QTL region according to a linkage disequilibrium r² value of at least 0.2 as threshold, wherein the highlighted range (including SNP A to SNP D) is the selected QTL region in accordance with the method of predicting palm oil yield of a test oil palm plant.

FIG. 4 is a plot of prediction accuracy (y-axis) versus number of SNP markers (x-axis) for the Ulu Remis dura×AVROS pisifera population, for SNP markers sorted based on their association score to the oil per palm plant (also termed O/P) trait, from high association to low association.

FIG. 5 is a plot of prediction accuracy (y-axis) versus number of SNP markers (x-axis) for the Banting dura×AVROS pisifera population, for SNP markers sorted based on their association score to the O/P trait, from high association to low association.

FIG. 6 is a plot of prediction accuracy (y-axis) versus number of SNP markers (x-axis) for the Ulu Remis dura (UR)×AVROS pisifera population (“⋄” diamond markers) and the Banting dura (BD)×AVROS pisifera population (“□” square markers), for SNP markers in linkage disequilibrium with SNP markers sorted based on their association score to the O/P trait, from high association to low association.

FIG. 7 is a plot of prediction accuracy (y-axis) versus number of SNP markers (x-axis) for the Ulu Remis dura (UR)×AVROS pisifera population (“⋄” diamond markers) and the Banting dura (BD)×AVROS pisifera population (“□” square markers), for a negative control corresponding to randomly selected SNP markers.

### BEST MODE FOR CARRYING OUT THE INVENTION

The application is drawn to methods for predicting palm oil yield of a test oil palm plant. The methods comprise steps of (i) determining, from a sample of a test oil palm plant of a population of oil palm plants, at least a first single nucleotide polymorphism (SNP) genotype of the test oil palm plant, (ii) comparing the first SNP genotype of the test oil palm plant to a corresponding first reference SNP genotype indicative of the high-oil-production trait in the same genetic background as the population, and (iii) predicting palm oil yield of the test oil palm plant based on the extent to which the first SNP genotype of the test oil palm plant matches the corresponding first reference SNP genotype. The first SNP genotype corresponds to a first SNP marker. The first SNP marker is located in a first quantitative trait locus (QTL) for a high-oil-production trait. The first SNP marker also is associated, after stratification and kinship correction, with the high-oil-production trait with a genome-wide −log₁₀(p-value) of at least 3.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 3.0 in the population. The first QTL is a region of the oil palm genome corresponding to one of QTL regions 1 to 57, as described in more detail below.

By conducting genome resequencing and genome-wide association studies of oil palm plants from two commercially relevant oil palm populations, including application of stratification and kinship correction, it has been determined that SNP markers that are located in 57 QTL regions of the oil palm genome and that are associated, after stratification and kinship correction, with a high-oil-production trait can be used to achieve 0.61 accuracy and 0.63 accuracy, measured in correlation, respectively, in the two populations. Moreover, by applying genomic selection, it has been determined that maximum prediction accuracy can be achieved by using approximately 500 SNP markers, sorted based on their association score to the oil per palm plant (also termed O/P) trait, from high association to low association.

Without wishing to be bound by theory, it is believed that identification of the 57 QTL regions and SNP markers therein that are associated, after stratification and kinship correction, with the high-oil-production trait will enable more rapid and efficient selection of candidate agricultural production palms and candidate breeding palms, from among the two commercially relevant oil palm populations and others. Stratification and kinship correction reduce false-positive signals due to recent common ancestry of small groups of individuals within the population of oil palm plants from which a test oil palm plant is sampled, thereby making practical the method for predicting palm oil yield of a test oil palm plant based on association. The methods will enable identification of potential high-yielding palms, for use in crosses to generate progeny with higher yields and for commercial production of palm oil, without need for cultivation of the palms to maturity, thus bypassing the need for the time and labor intensive cultivations and measurements, the destructive sampling of fruits, and the impracticality of direct hybrid crosses that are characteristic of conventional approaches. For example, the methods can be used to choose oil palm plants for germination, cultivation in a nursery, cultivation for commercial production of palm oil, cultivation for further propagation, etc., well before direct measurement of palm oil production by the test oil palm plant could be accomplished. Also for example, the methods can be used to accomplish prediction of palm oil yields with greater efficiency and/or less variability than by direct measurement of palm oil production. The methods can be used advantageously with respect to even a single SNP, given that improvements in palm oil yield that seem small on a percentage basis still can have a dramatic effect on overall palm oil yields, given the large scale of commercial cultivations. The methods also can be used advantageously with respect to combinations of two or more SNPs, e.g. a first SNP genotype and a second SNP genotype, or a first SNP genotype to a fifty-seventh SNP genotype, given additive and/or synergistic effects.

The terms “high-oil-production trait,” “high yield,” “high-yielding,” and “oil yield,” as used with respect to the methods and kits disclosed herein, refer to yields of palm oil in mesocarp tissue of fruits of palm oil plants.

The singular forms “a,” “an,” and “the” are intended to include the plural forms as well, unless the context clearly indicates otherwise.

As noted above, a method for predicting palm oil yield of a test oil palm plant is disclosed. The method comprises a step of (i) determining, from a sample of a test oil palm plant of a population of oil palm plants, at least a first single nucleotide polymorphism (also termed SNP) genotype of the test oil palm plant.

The SNP genotype of the test oil palm plant corresponds to the constitution of SNP alleles at a particular locus, or position, on each chromosome in which the locus occurs in the genome of the test oil palm plant. A SNP is a polymorphic variation with respect to a single nucleotide that occurs at such a locus on a chromosome. A SNP allele is the specific nucleotide present at the locus on the chromosome. For oil palm plants, which are diploid and which thus inherit one set of maternally derived chromosomes and one set of paternally derived chromosomes, the SNP genotype corresponds to two SNP alleles, one at the particular locus on the maternally derived chromosome and the other at the particular locus on the paternally derived chromosome. Each SNP allele may be classified, for example, based on allele frequency, e.g. as a major allele (A) or a minor allele (a). Thus, for example, the SNP genotype can correspond to two major alleles (A/A), one major allele and one minor allele (A/a), or two minor alleles (a/a).

The test oil palm plant can be an oil palm plant corresponding to an important oil-food crop. For example, the test oil palm plant can correspond to African oil palm Elaeis guineensis.

The test oil palm plant can be an oil palm plant in any suitable form. For example, the test oil palm plant can be a seed, a seedling, a nursery phase plant, an immature phase plant, a cell culture plant, a zygotic embryo culture plant, or a somatic tissue culture plant. Also for example, the test oil palm plant can be a production phase plant, a mature palm, a mature mother palm, or a mature pollen donor.

A test oil palm plant in the form of a seed, a seedling, a nursery phase plant, an immature phase plant, a cell culture plant, a zygotic embryo culture plant, or a somatic tissue culture plant is in a form that is not yet mature, and thus that is not yet producing palm oil in amounts typical of commercial production, if at all. Accordingly, the method as applied to a test oil palm plant in such a form can be used to predict palm oil yield of the test oil palm plant before the test oil palm plant has matured sufficiently to allow direct measurement of palm oil production by the test oil palm plant during commercial production.

A test oil palm plant in the form of a production phase plant, a mature palm, a mature mother palm, or a mature pollen donor is in a form that is mature. Accordingly, the method as applied to a test oil palm plant in such a form can be used to predict palm oil yield of the test oil palm as an alternative to direct measurement of palm oil yield.

The population of oil palm plants from which the test oil palm plant is sampled can comprise any suitable population of oil palm plants. The population can be specified in terms of fruit type and/or identity of the breeding material from which the population was generated. In this regard, fruit type is a monogenic trait in oil palm that is important with respect to breeding and commercial production. Oil palms with either of two distinct fruit types are generally used in breeding and seed production through crossing in order to generate palms for commercial production of palm oil, also termed commercial planting materials or agricultural production plants. The first fruit type is dura (genotype: sh+ sh+), which is characterized by a thick shell corresponding to 28 to 35% of the fruit by weight, with no ring of black fibres around the kernel of the fruit. For dura fruits, the ratio of mesocarp to fruit varies from 50 to 60%, with extractable oil content in proportion to bunch weight of 18 to 24%. The second fruit type is pisifera (genotype: sh− sh−), which is characterized by the absence of a shell, the vestiges of which are represented by a ring of fibres around a small kernel. Accordingly, for pisifera fruits, the ratio of mesocarp to fruit is 90 to 100%. The ratio of mesocarp oil to bunch is comparable to the dura at 16 to 28%. Pisiferas are however usually female sterile as the majority of bunches abort at an early stage of development.

Crossing dura and pisifera gives rise to palms with a third fruit type, the tenera (genotype: sh+ sh−). Tenera fruits have thin shells of 8 to 10% of the fruit by weight, corresponding to a thickness of 0.5 to 4 mm, around which is a characteristic ring of black fibres. For tenera fruits, the ratio of mesocarp to fruit is comparatively high, in the range of 60 to 80%. Commercial tenera palms generally produce more fruit bunches than duras, although mean bunch weight is lower. The ratio of mesocarp oil to bunch is in the range of 20 to 30%, the highest of the three fruit types, and thus tenera are typically used as commercial planting materials.

Identity of the breeding material can be based on the source and breeding history of the breeding material. Dura palm breeding populations used in Southeast Asia include Serdang Avenue, Ulu Remis (which incorporated some Serdang Avenue material), Banting, Johor Labis, and Elmina estate, including Deli Dumpy, all of which are derived from Deli dura. Pisifera breeding populations used for seed production are generally grouped as Yangambi, AVROS, Binga and URT. Other dura and pisifera populations are used in Africa and South America.

Oil palm plantation/breeding programs in Southeast Asia are using Deli dura origin, which originated from the four famous dura palms at Bogor in the year 1848. The Deli dura materials were subsequently distributed to several research stations across the region. Each station focused on different selection preferences over generations, leading to some differentiation between subpopulations, termed breeding populations of restricted origin (also termed “BPRO”). The important breeding populations of restricted origin derived from Deli dura are Ulu Remis (also termed “UR”) and Johor Labis (also termed “JL”). The Ulu Remis origin was selected for high bunch number and high sex ratio (defined as ratio of females to total inflorescences) in Marihat Baris, Sumatra. Instead of bunch number, Socfindo in Sumatra had developed Johor Labis origin for bigger bunches (high bunch weight) and thinner shells.

Dura palms were commercially planted in Southeast Asia before the 1960's. The Banting dura (also termed “BD”) was discovered in the commercial Deli dura planted in 1958 in Dusun Durian Estate. The material was selected for good bunch traits and number. Banting dura has become an important maternal source.

African dura materials are inferior to Deli dura. To increase oil yield, the main planting materials in Africa were tenera (dura×pisifera). This provided an opportunity to discover a superior pollen source, i.e. AVROS pisifera. The material originated from the renowned Djongo palms that were planted in Eala Botanical Garden in Yangambi, Zaire, now the Democratic Republic of the Congo. The material was then further selected and produced BM119 at Kelanang Bharu Division of Dusun Durian Estate. The AVROS pisifera confers superiority in growth uniformity, general combining ability, precocity, and mesocarp oil yield in Deli×AVROS progeny (tenera). Thus, the introduction of Deli dura×BM119 AVROS pisifera in the region resulted in an increase in oil per hectare of 30% since the 1960's

Oil palm breeding is primarily aimed at selecting for improved parental dura and pisifera breeding stock palms for production of superior tenera commercial planting materials. Such materials are largely in the form of seeds although the use of tissue culture for propagation of clones continues to be developed. Generally, parental dura breeding populations are generated by crossing among selected dura palms. Based on the monogenic inheritance of fruit type, 100% of the resulting palms will be duras. After several years of yield recording and confirmation of bunch and fruit characteristics, duras are selected for breeding based on phenotype. In contrast, pisifera palms are normally female sterile and thus breeding populations thereof must be generated by crossing among selected teneras or by crossing selected teneras with selected pisiferas. The tenera×tenera cross will generate 25% duras, 50% teneras and 25% pisiferas. The tenera×pisifera cross will generate 50% teneras and 50% pisiferas. The yield potential of pisiferas is then determined indirectly by progeny testing with the elite duras, i.e. by crossing duras and pisiferas to generate teneras, and then determining yield phenotypes of the fruits of the teneras over time. From this, pisiferas with good general combining ability are selected based on the performance of their tenera progenies. Intercrossing among selected parents is also carried out with progenies being carried forward to the next breeding cycle. This allows introduction of new genes into the breeding programme to increase genetic variability.

Oil palm cultivation for commercial production of palm oil can be improved by use of the superior tenera commercial planting materials. Priority selection objectives include high oil yield per unit area in terms of high fresh fruit bunch (also termed FFB) yield and high oil to bunch ratio (also termed O/B) (thin shell, thick mesocarp), high early yield (precocity), and good oil qualities, among other traits. Progeny plants may be cultivated by conventional approaches, e.g. seedlings may be cultivated in polyethylene bags in pre-nursery and nursery settings, raised for about 12 months, and then planted as seedlings, with progeny that are known or predicted to exhibit high yields chosen for further cultivation, among other approaches.

Accordingly, in some examples the population of oil palm plants comprises an Ulu Remis dura×AVROS pisifera population, a Banting dura×AVROS pisifera population, or a combination thereof. Also in some examples the population of oil palm plants comprises an Ulu Remis dura×Ulu Remis dura population, an Ulu Remis dura×Banting dura population, a Banting dura×Banting dura population, an AVROS pisifera×AVROS tenera population, an AVROS tenera×AVROS tenera population, or a combination thereof.

The sample of the test oil palm plant can comprise any organ, tissue, cell, or other part of the test oil palm plant that includes sufficient genomic DNA of the test oil palm plant to allow for determination of one or more SNP genotypes of the test oil palm plant, e.g. the first SNP genotype. For example, the sample can comprise a leaf tissue, among other organs, tissues, cells, or other parts. As one of ordinary skill will appreciate, determining, from a sample of a test oil palm plant, one or more SNP genotypes of the test oil palm plant, is necessarily transformative of the sample. The one or more SNP genotypes cannot be determined, for example, merely based on appearance of the sample. Rather, determination of the one or more SNP genotypes of the test oil palm plant requires separation of the sample from the test oil palm plant and/or separation of genomic DNA from the sample.

Determination of the at least first SNP genotype can be carried out by any suitable technique, including, for example, whole genome resequencing with SNP calling, hybridization-based methods, enzyme-based methods, or other post-amplification methods, among others.

The first SNP genotype corresponds to a first SNP marker. A SNP marker is a SNP that can be used in genetic mapping.

The first SNP marker is located in a first quantitative trait locus (also termed QTL) for a high-oil-production trait. A QTL is a locus extending along a portion of a chromosome that contributes in determining a phenotype of a continuous character, i.e. in this case, the high-oil-production trait.

The high-oil-production trait relates to a trait of production of palm oil by the test oil palm plant upon reaching a mature state, e.g. reaching production phase, and upon being cultivated under conditions suitable for production of palm oil in a high amount, e.g. commercial cultivation, in an amount that is higher than average, with respect to the population of oil palm plants from which the test oil palm plant is sampled, also upon reaching a mature state and upon being cultivated under conditions suitable for production of palm oil in a high amount.

Considering a test oil plant that is a tenera oil palm plant, the high-oil-production trait can correspond, for example, to production of palm oil at greater than 3.67 tonnes of palm oil per hectare per year, i.e. above recent average yields for typical oil palm plants used in commercial production, which also are tenera oil palm plants, as discussed above. The high-oil production trait also can correspond, for example, to production of palm oil at greater than 10 tonnes of palm oil per hectare per year, i.e. above recent average yields for current best-progeny oil palm plants used in commercial production. The high-oil production trait also can correspond, for example, to production of palm oil at greater than 4, 5, 6, 7, 8, or 9 tonnes of palm oil per hectare per year, i.e. above yields that are intermediate between the recent average yields noted above. Considering a test oil palm plant that is a dura oil palm plant or a pisifera oil palm plant, the high-oil production trait can correspond to production of palm oil in correspondingly lower amounts, consistent with lower average yields obtained for dura and pisifera oil palm plants relative to tenera oil palm plants.

The high-oil-production trait can comprise increased oil per palm plant (also termed O/P). As noted above, palm oil is produced in the mesocarp of the oil palm fruit. O/P is a measure of palm oil yield. Accordingly, a relatively high 0/P is an indicator of relatively high production of palm oil.

The first SNP marker is associated, after stratification and kinship correction, with the high-oil-production trait with a genome-wide −log₁₀(p-value) of at least 3.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 3.0 in the population.

A first SNP marker being associated, after stratification and kinship correction, with a trait with a genome-wide −log₁₀(p-value) of at least 3.0 in a population indicates that a high likelihood exists that the first SNP maker and the trait are associated.

A p-value is the probability of observing a test statistic, in this case relating to association of a SNP marker, e.g. the first SNP marker or the first other SNP marker, and the high-oil-production trait, equal to or greater than a test statistic actually observed, if the null hypothesis is true and thus there is no association, as discussed, for example, by Bush & Moore, Chapter 11: Genome-Wide Association Studies, PLOS Computational Biology 8(12):e1002822, 1-11 (2012). A genome-wide −log₁₀(p-value) corresponds to a p-value expressed on a logarithmic scale, for convenience, and corrected to take into account the effective number of statistical tests that have been carried out, based on multiple tests for association conducted with respect to an entire genome of a corresponding specific population, also as discussed by Bush & Moore (2012). Accordingly, a genome-wide −log₁₀(p-value) that is relatively high indicates that the likelihood that the observed test statistic, relating to association, would have been observed in the absence of association is extremely low.

Stratification and kinship correction are taken into account in determining the association. As noted above, stratification and kinship correction reduce false-positive signals due to recent common ancestry of small groups of individuals within the population of oil palm plants from which the test oil palm plant is sampled, thereby making practical the method for predicting palm oil yield of a test oil palm plant based on association.

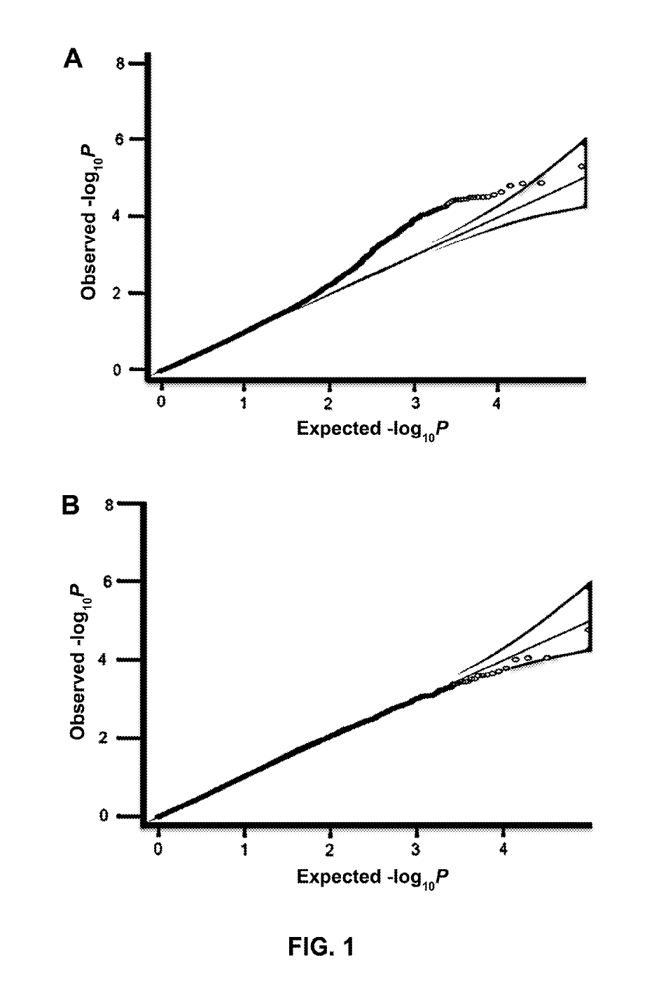

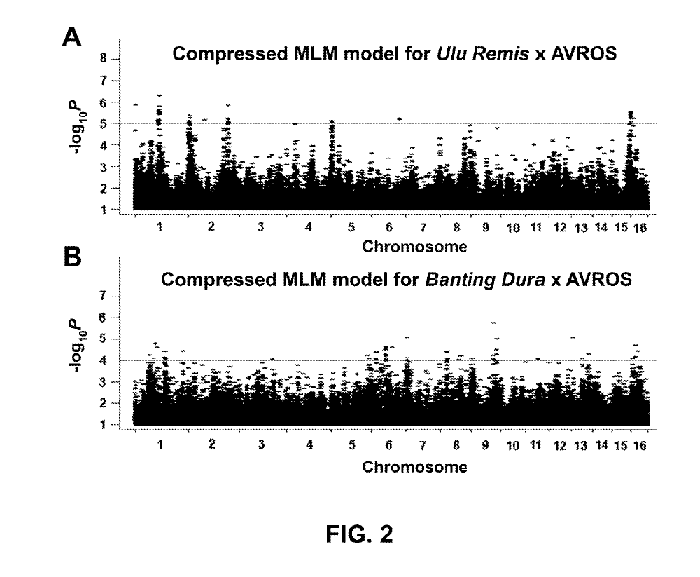

For reference, a genome-wide association study (also termed GWAS) was performed on Ulu Remis×AVROS and Banting Dura×AVROS, which are commercially relevant oil palm populations, respectively using a compressed mixed linear model (also termed MLM) with population parameters previously determined (P3D), to address the problem of genomic inflations using group kinship matrix. Specifically, as shown in FIG. 1, the Q-Q plots in both populations showed that deviation of the observed statistics from the null expectation were delayed significantly. As shown in FIG. 2, the chromosomal distribution of the resulting SNPs for both populations can be visualized in Manhattan plots. Based on this approach, a total of 119 SNPs that are informative with respect to O/P were identified after excluding markers that overlapped in both populations.

Stratification and kinship correction can be applied similarly regarding other oil palm populations, e.g. the populations noted above.

Accordingly, for example, the first SNP marker being located in a first QTL for a high-oil-production trait and being associated, after stratification and kinship correction, with the high-oil-production trait with a genome-wide −log₁₀(p-value) of at least 3.0 in the population can be a SNP marker for which association with the high-oil-production trait (i) has been confirmed based on a model that is not a naive model and/or (ii) would be confirmed based on a model that is not a naive model. Also for example, the first SNP marker being located in a first QTL for a high-oil-production trait and being associated, after stratification and kinship correction, with the high-oil-production trait with a genome-wide −log₁₀(p-value) of at least 3.0 in the population can be a SNP marker for which association with the high-oil-production trait (i) has been confirmed based on a compressed mixed linear model with population parameters previously determined, carried out using principal component analysis and a group kinship matrix and/or (ii) would be confirmed based on a compressed mixed linear model with population parameters previously determined, carried out using principal component analysis and a group kinship matrix.

A first SNP marker having a linkage disequilibrium r² value of at least 0.2 with respect to a first other SNP marker that is linked thereto and associated, after stratification and kinship correction, with the high-oil-production trait with a genome-wide −log₁₀(p-value) of at least 3.0 in the population indicates the following. First, a high likelihood exists that an allele of the first SNP marker and an allele of the first other SNP marker are in linkage disequilibrium. Second, a high likelihood exists that the first other SNP marker and the trait are associated. In this regard, a linkage disequilibrium r² value relates to measuring likelihood that two loci are in linkage disequilibrium as an average pairwise correlation coefficient.

Accordingly, in some examples the first SNP marker is associated, after stratification and kinship correction, with the high-oil-production trait with a genome-wide −log₁₀(p-value) of at least 3.0 in the population. Also, in some examples the first SNP marker has a linkage disequilibrium r² value of at least 0.2 with respect to a first other SNP marker that is linked thereto and associated, after stratification and kinship correction, with the high-oil-production trait with a genome-wide −log₁₀(p-value) of at least 3.0 in the population. Also, in some examples both apply.

The first QTL can be a region of the oil palm genome corresponding to one of:

(1) QTL region 1, extending from nucleotide 18204491 to 18358401 of chromosome 1;
(2) QTL region 2, extending from nucleotide 18922390 to 19167923 of chromosome 1;
(3) QTL region 3, extending from nucleotide 19188077 to 19685080 of chromosome 1;
(4) QTL region 4, extending from nucleotide 23276098 to 23456770 of chromosome 1;
(5) QTL region 5, extending from nucleotide 26021716 to 26066534 of chromosome 1;
(6) QTL region 6, extending from nucleotide 28110016 to 28234799 of chromosome 1;
(7) QTL region 7, extending from nucleotide 29798161 to 30164329 of chromosome 1;
(8) QTL region 8, extending from nucleotide 30684639 to 31160129 of chromosome 1;
(9) QTL region 9, extending from nucleotide 37811723 to 38637229 of chromosome 1;
(10) QTL region 10, extending from nucleotide 38659012 to 39206652 of chromosome 1;
(11) QTL region 11, extending from nucleotide 39243858 to 39842157 of chromosome 1;
(12) QTL region 12, extending from nucleotide 61305818 to 61572106 of chromosome 1;
(13) QTL region 13, extending from nucleotide 1068379 to 1516571 of chromosome 2;
(14) QTL region 14, extending from nucleotide 1616491 to 2016169 of chromosome 2;
(15) QTL region 15, extending from nucleotide 17637996 to 17959911 of chromosome 2;
(16) QTL region 16, extending from nucleotide 20732085 to 20977490 of chromosome 2;
(17) QTL region 17, extending from nucleotide 31844836 to 31980071 of chromosome 2;
(18) QTL region 18, extending from nucleotide 50449700 to 50857310 of chromosome 2;
(19) QTL region 19, extending from nucleotide 50879601 to 51539414 of chromosome 2;
(20) QTL region 20, extending from nucleotide 52821582 to 52960520 of chromosome 2;
(21) QTL region 21, extending from nucleotide 42585292 to 42728875 of chromosome 3;
(22) QTL region 22, extending from nucleotide 9561644 to 9701199 of chromosome 4;
(23) QTL region 23, extending from nucleotide 12469969 to 13409114 of chromosome 4;
(24) QTL region 24, extending from nucleotide 14672228 to 14789226 of chromosome 4;
(25) QTL region 25, extending from nucleotide 395189 to 842107 of chromosome 5;
(26) QTL region 26, extending from nucleotide 47205529 to 47293291 of chromosome 5;
(27) QTL region 27, extending from nucleotide 48857594 to 48932286 of chromosome 5;
(28) QTL region 28, extending from nucleotide 5943980 to 6002717 of chromosome 6;
(29) QTL region 29, extending from nucleotide 6337822 to 6563232 of chromosome 6;
(30) QTL region 30, extending from nucleotide 6818733 to 7281658 of chromosome 6;
(31) QTL region 31, extending from nucleotide 17578027 to 18209857 of chromosome 6;
(32) QTL region 32, extending from nucleotide 26204516 to 26755007 of chromosome 6;
(33) QTL region 33, extending from nucleotide 36492757 to 36494757 of chromosome 6;
(34) QTL region 34, extending from nucleotide 219790 to 1533149 of chromosome 7;
(35) QTL region 35, extending from nucleotide 8700733 to 9242332 of chromosome 8;
(36) QTL region 36, extending from nucleotide 23767318 to 23957652 of chromosome 8;
(37) QTL region 37, extending from nucleotide 26648547 to 26848102 of chromosome 8;
(38) QTL region 38, extending from nucleotide 606020 to 1309231 of chromosome 9;
(39) QTL region 39, extending from nucleotide 3499347 to 3638435 of chromosome 9;
(40) QTL region 40, extending from nucleotide 28437588 to 28513671 of chromosome 9;
(41) QTL region 41, extending from nucleotide 28581068 to 28912034 of chromosome 9;
(42) QTL region 42, extending from nucleotide 32327318 to 32434321 of chromosome 9;
(43) QTL region 43, extending from nucleotide 32538074 to 32540074 of chromosome 9;
(44) QTL region 44, extending from nucleotide 32775289 to 33054696 of chromosome 9;
(45) QTL region 45, extending from nucleotide 33133902 to 33254107 of chromosome 9;
(46) QTL region 46, extending from nucleotide 15342814 to 15405953 of chromosome 10;
(47) QTL region 47, extending from nucleotide 15933273 to 15943963 of chromosome 11;
(48) QTL region 48, extending from nucleotide 12178551 to 12249693 of chromosome 12;
(49) QTL region 49, extending from nucleotide 2052746 to 2447722 of chromosome 13;
(50) QTL region 50, extending from nucleotide 14345084 to 14709650 of chromosome 13;
(51) QTL region 51, extending from nucleotide 22031000 to 22147560 of chromosome 13;
(52) QTL region 52, extending from nucleotide 23588504 to 24307350 of chromosome 15;
(53) QTL region 53, extending from nucleotide 1511530 to 1596020 of chromosome 16;
(54) QTL region 54, extending from nucleotide 2684531 to 2803682 of chromosome 16;
(55) QTL region 55, extending from nucleotide 5535711 to 5995857 of chromosome 16;
(56) QTL region 56, extending from nucleotide 8379248 to 8554851 of chromosome 16; or
(57) QTL region 57, extending from nucleotide 8883687 to 9269845 of chromosome 16.

The numbering of chromosomes, also termed linkage groups, and nucleotides thereof is in accordance with a 1.8 gigabase genome sequence of the African oil palm E. guineensis as described by Singh et al., Nature 500:335-339 (2013) and the supplementary information noted therein, indicating that the E. guineensis BioProject is available for download at http://genomsawit.mpob.gov.my and has been registered at the NCBI under BioProject accession PRJNA192219 and that the Whole Genome Shotgun project has been deposited at DDBJ/EMBL/GenBank under the accession ASJS00000000.

For reference, QTL region 1 corresponds to the region of chromosome 1 of the genome of oil palm extending from the 5′ end of SEQ ID NO: 1 to the 3′ end of SEQ ID NO: 2. Similarly, QTL region 2 corresponds to the region of chromosome 1 extending from the 5′ end of SEQ ID NO: 3 to the 3′ end of SEQ ID NO: 4. QTL region 3 corresponds to the region of chromosome 1 extending from the 5′ end of SEQ ID NO: 5 to the 3′ end of SEQ ID NO: 6. QTL region 4 corresponds to the region of chromosome 1 extending from the 5′ end of SEQ ID NO: 7 to the 3′ end of SEQ ID NO: 8. QTL region 5 corresponds to the region of chromosome 1 extending from the 5′ end of SEQ ID NO: 9 to the 3′ end of SEQ ID NO: 10. QTL region 6 corresponds to the region of chromosome 1 extending from the 5′ end of SEQ ID NO: 11 to the 3′ end of SEQ ID NO: 12. QTL region 7 corresponds to the region of chromosome 1 extending from the 5′ end of SEQ ID NO: 13 to the 3′ end of SEQ ID NO: 14. QTL region 8 corresponds to the region of chromosome 1 extending from the 5′ end of SEQ ID NO: 15 to the 3′ end of SEQ ID NO: 16. QTL region 9 corresponds to the region of chromosome 1 extending from the 5′ end of SEQ ID NO: 17 to the 3′ end of SEQ ID NO: 18. QTL region 10 corresponds to the region of chromosome 1 extending from the 5′ end of SEQ ID NO: 19 to the 3′ end of SEQ ID NO: 20. QTL region 11 corresponds to the region of chromosome 1 extending from the 5′ end of SEQ ID NO: 21 to the 3′ end of SEQ ID NO: 22. QTL region 12 corresponds to the region of chromosome 1 extending from the 5′ end of SEQ ID NO: 23 to the 3′ end of SEQ ID NO: 24. QTL region 13 corresponds to the region of chromosome 2 extending from the 5′ end of SEQ ID NO: 25 to the 3′ end of SEQ ID NO: 26. QTL region 14 corresponds to the region of chromosome 2 extending from the 5′ end of SEQ ID NO: 27 to the 3′ end of SEQ ID NO: 28. QTL region 15 corresponds to the region of chromosome 2 extending from the 5′ end of SEQ ID NO: 29 to the 3′ end of SEQ ID NO: 30. QTL region 16 corresponds to the region of chromosome 2 extending from the 5′ end of SEQ ID NO: 31 to the 3′ end of SEQ ID NO: 32. QTL region 17 corresponds to the region of chromosome 2 extending from the 5′ end of SEQ ID NO: 33 to the 3′ end of SEQ ID NO: 34. QTL region 18 corresponds to the region of chromosome 2 extending from the 5′ end of SEQ ID NO: 35 to the 3′ end of SEQ ID NO: 36. QTL region 19 corresponds to the region of chromosome 2 extending from the 5′ end of SEQ ID NO: 37 to the 3′ end of SEQ ID NO: 38. QTL region 20 corresponds to the region of chromosome 2 extending from the 5′ end of SEQ ID NO: 39 to the 3′ end of SEQ ID NO: 40. QTL region 21 corresponds to the region of chromosome 3 extending from the 5′ end of SEQ ID NO: 41 to the 3′ end of SEQ ID NO: 42. QTL region 22 corresponds to the region of chromosome 4 extending from the 5′ end of SEQ ID NO: 43 to the 3′ end of SEQ ID NO: 44. QTL region 23 corresponds to the region of chromosome 4 extending from the 5′ end of SEQ ID NO: 45 to the 3′ end of SEQ ID NO: 46. QTL region 24 corresponds to the region of chromosome 4 extending from the 5′ end of SEQ ID NO: 47 to the 3′ end of SEQ ID NO: 48. QTL region 25 corresponds to the region of chromosome 5 extending from the 5′ end of SEQ ID NO: 49 to the 3′ end of SEQ ID NO: 50. QTL region 26 corresponds to the region of chromosome 5 extending from the 5′ end of SEQ ID NO: 51 to the 3′ end of SEQ ID NO: 52. QTL region 27 corresponds to the region of chromosome 5 extending from the 5′ end of SEQ ID NO: 53 to the 3′ end of SEQ ID NO: 54. QTL region 28 corresponds to the region of chromosome 6 extending from the 5′ end of SEQ ID NO: 55 to the 3′ end of SEQ ID NO: 56. QTL region 29 corresponds to the region of chromosome 6 extending from the 5′ end of SEQ ID NO: 57 to the 3′ end of SEQ ID NO: 58. QTL region 30 corresponds to the region of chromosome 6 extending from the 5′ end of SEQ ID NO: 59 to the 3′ end of SEQ ID NO: 60. QTL region 31 corresponds to the region of chromosome 6 extending from the 5′ end of SEQ ID NO: 61 to the 3′ end of SEQ ID NO: 62. QTL region 32 corresponds to the region of chromosome 6 extending from the 5′ end of SEQ ID NO: 63 to the 3′ end of SEQ ID NO: 64. QTL region 33 corresponds to the region of chromosome 6 extending from the 5′ end of SEQ ID NO: 65 to the 3′ end of SEQ ID NO: 66. QTL region 34 corresponds to the region of chromosome 7 extending from the 5′ end of SEQ ID NO: 67 to the 3′ end of SEQ ID NO: 68. QTL region 35 corresponds to the region of chromosome 8 extending from the 5′ end of SEQ ID NO: 69 to the 3′ end of SEQ ID NO: 70. QTL region 36 corresponds to the region of chromosome 8 extending from the 5′ end of SEQ ID NO: 71 to the 3′ end of SEQ ID NO: 72. QTL region 37 corresponds to the region of chromosome 8 extending from the 5′ end of SEQ ID NO: 73 to the 3′ end of SEQ ID NO: 74. QTL region 38 corresponds to the region of chromosome 9 extending from the 5′ end of SEQ ID NO: 75 to the 3′ end of SEQ ID NO: 76. QTL region 39 corresponds to the region of chromosome 9 extending from the 5′ end of SEQ ID NO: 77 to the 3′ end of SEQ ID NO: 78. QTL region 40 corresponds to the region of chromosome 9 extending from the 5′ end of SEQ ID NO: 79 to the 3′ end of SEQ ID NO: 80. QTL region 41 corresponds to the region of chromosome 9 extending from the 5′ end of SEQ ID NO: 81 to the 3′ end of SEQ ID NO: 82. QTL region 42 corresponds to the region of chromosome 9 extending from the 5′ end of SEQ ID NO: 83 to the 3′ end of SEQ ID NO: 84. QTL region 43 corresponds to the region of chromosome 9 extending from the 5′ end of SEQ ID NO: 85 to the 3′ end of SEQ ID NO: 86. QTL region 44 corresponds to the region of chromosome 9 extending from the 5′ end of SEQ ID NO: 87 to the 3′ end of SEQ ID NO: 88. QTL region 45 corresponds to the region of chromosome 9 extending from the 5′ end of SEQ ID NO: 89 to the 3′ end of SEQ ID NO: 90. QTL region 46 corresponds to the region of chromosome 10 extending from the 5′ end of SEQ ID NO: 91 to the 3′ end of SEQ ID NO: 92. QTL region 47 corresponds to the region of chromosome 11 extending from the 5′ end of SEQ ID NO: 93 to the 3′ end of SEQ ID NO: 94. QTL region 48 corresponds to the region of chromosome 12 extending from the 5′ end of SEQ ID NO: 95 to the 3′ end of SEQ ID NO: 96. QTL region 49 corresponds to the region of chromosome 13 extending from the 5′ end of SEQ ID NO: 97 to the 3′ end of SEQ ID NO: 98. QTL region 50 corresponds to the region of chromosome 13 extending from the 5′ end of SEQ ID NO: 99 to the 3′ end of SEQ ID NO: 100. QTL region 51 corresponds to the region of chromosome 13 extending from the 5′ end of SEQ ID NO: 101 to the 3′ end of SEQ ID NO: 102. QTL region 52 corresponds to the region of chromosome 15 extending from the 5′ end of SEQ ID NO: 103 to the 3′ end of SEQ ID NO: 104. QTL region 53 corresponds to the region of chromosome 16 extending from the 5′ end of SEQ ID NO: 105 to the 3′ end of SEQ ID NO: 106. QTL region 54 corresponds to the region of chromosome 16 extending from the 5′ end of SEQ ID NO: 107 to the 3′ end of SEQ ID NO: 108. QTL region 55 corresponds to the region of chromosome 16 extending from the 5′ end of SEQ ID NO: 109 to the 3′ end of SEQ ID NO: 110. QTL region 56 corresponds to the region of chromosome 16 extending from the 5′ end of SEQ ID NO: 111 to the 3′ end of SEQ ID NO: 112. QTL region 57 corresponds to the region of chromosome 16 extending from the 5′ end of SEQ ID NO: 113 to the 3′ end of SEQ ID NO: 114.

The method also comprises a step of (ii) comparing the first SNP genotype of the test oil palm plant to a corresponding first reference SNP genotype indicative of the high-oil-production trait in the same genetic background as the population. The genetic background that is the same as the population can correspond, for example, to a population based on crossing oil palm plants of the same types as used to generate the population from which the test oil palm plant is sampled, e.g. an Ulu Remis dura×AVROS pisifera population, a Banting dura×AVROS pisifera population, or a combination thereof, or an Ulu Remis dura×Ulu Remis dura population, an Ulu Remis dura×Banting dura population, a Banting dura×Banting dura population, an AVROS pisifera×AVROS tenera population, an AVROS tenera×AVROS tenera population, or a combination thereof. The genetic background that is the same as the population also can correspond, for example, to a population based on crossing the same individual oil palm plants used to generate the population from which the test oil palm plant is sampled. The genetic background that is the same as the population also can correspond, for example, to the same actual population from which the test oil palm plant is sampled.

The first reference SNP genotype indicative of the high-oil-production trait in the same genetic background as the population can correspond to the same SNP as the first SNP genotype, i.e. both can correspond to the same polymorphic variation with respect to a single nucleotide that occurs at a particular locus of a particular chromosome. The first reference SNP genotype can comprise one or more SNP alleles that, alone or together, indicate a higher likelihood that the test oil palm plant thereof exhibits, if mature, or will exhibit, upon reaching maturity, the high-oil-production trait, in comparison to oil palm plants of the same population that lack the one or more SNP alleles.

The method also comprises a step of (iii) predicting palm oil yield of the test oil palm plant based on the extent to which the first SNP genotype of the test oil palm plant matches the corresponding first reference SNP genotype. The first SNP genotype of the test oil palm plant can match the corresponding first reference SNP genotype based on both SNP genotypes sharing at least a first SNP allele indicative of the high-oil-production trait in the same genetic background as the population. In some examples the first SNP genotype and the first reference SNP genotype are heterozygous for the first allele indicative of the high-oil production trait, i.e. both have only one copy of the SNP allele. Also, in some examples the first SNP genotype and the first reference SNP genotype are homozygous for the first allele indicative of the high-oil production trait, i.e. both have two copies of the SNP allele. Also, in some examples the first SNP genotype is heterozygous for the first allele indicative of the high-oil production trait and the first reference SNP genotype is homozygous for the first allele indicative of the high-oil production trait. Also, in some examples the first SNP genotype is homozygous for the first allele indicative of the high-oil production trait and the first reference SNP genotype is heterozygous for the first allele indicative of the high-oil production trait.

The step of predicting palm oil yield of the test oil palm plant can further comprise applying a model, such as a genotype model, a dominant model, or a recessive model, among others, in order to facilitate the predicting. A genotype model tests the association of a trait, e.g. a high-oil production trait, with the presence of a SNP allele, either a major allele (A) or a minor allele (a). A dominant model tests the association of a trait, e.g. a high-oil production trait, with the presence of a SNP allele either as a homozygous genotype or a heterozygous genotype, e.g. the major allele either as a homozygous genotype (e.g. A/A) or a heterozygous genotype (e.g. A/a). A recessive model tests the association of a trait, e.g. a high-oil production trait, with the presence of a SNP allele as a homozygous genotype, e.g. the minor allele as a homozygous genotype (a/a). Accordingly, in some examples, the predicting of palm oil yield of the test oil palm plant further comprises applying a genotype model. Also in some examples, the predicting of palm oil yield of the test oil palm plant further comprises applying a dominant model. Also in some examples, the predicting of palm oil yield of the test oil palm plant further comprises applying a recessive model.

The degree to which a particular SNP genotype of a SNP marker in QTL regions 1 to 57 can be useful for predicting palm oil yield of a test oil palm plant can depend on the source and breeding history of the breeding materials used to generate the population from which the test oil palm is sampled, including for example the extent to which one or more high-yield variant alleles that result in increases in palm oil yield have arisen within QTL regions 1 to 57 of the breeding materials and/or sources thereof used to generate the population, as well as the proximity of the one or more high-yield variant alleles to SNPs and the extent to which recombination has occurred between the SNPs and the high-yield variant alleles since the high-yield variant alleles arose. Factors such as proximity between a high-yield variant allele that promotes a high-oil-production trait and a SNP allele, a low number of generations since the high-yield variant allele arose, and a strong positive effect of the high-yield variant allele on palm oil production can tend to increase the degree to which a particular SNP can be informative. These factors can vary, for example, depending on whether a high-yield variant allele is dominant or recessive, and thus whether a genotype model, a dominant model, or a recessive model may appropriately be applied with respect to a corresponding SNP allele. These factors also can vary, for example, between different populations generated by crosses of different individual palm plants.

The step of predicting palm oil yield of the test oil palm plant can be used advantageously not just to predict the palm oil yield of the test oil palm plant itself, but also to predict palm oil yields of progeny thereof. In this regard, oil palm breeders can use the method, as applied to a test oil palm plant that is a mother palm or a pollen donor, to determine possible SNP genotypes of progeny to be generated by crossing the test oil palm plant with another oil palm plant, and moreover can choose specific palms, i.e. the test oil palm plant and another specific oil palm plant that has been similarly characterized, to be crossed on this basis.

The method for predicting palm oil yield of a test oil palm plant can be used by focusing on particular QTLs, or combinations thereof, with respect to test oil palm plants derived from particular breeding materials.

For example, in some examples the population of oil palm plants comprises an Ulu Remis dura×AVROS pisifera population, the first QTL corresponds to one of QTL regions 7, 8, 13, 14, 16, 18, 19, 25, 33, 52, or 54, step (iii) further comprises applying a genotype model, thereby predicting the palm oil yield of the test oil palm plant, and the first SNP marker is associated, after stratification and kinship correction, with the high-oil-production trait with a genome-wide −log₁₀(p-value) of at least 4.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 4.0 in the population.

Also, in some examples the population of oil palm plants comprises an Ulu Remis dura×AVROS pisifera population, the first QTL corresponds to QTL region 8, step (iii) further comprises applying a dominant model, thereby predicting the palm oil yield of the test oil palm plant, and the first SNP marker is associated, after stratification and kinship correction, with the high-oil-production trait with a genome-wide −log₁₀(p-value) of at least 4.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 4.0 in the population.

Also in some examples the population of oil palm plants comprises an Ulu Remis dura×AVROS pisifera population, the first QTL corresponds to one of QTL regions 8, 13, 18, 22, 23, or 45, step (iii) further comprises applying a recessive model, thereby predicting the palm oil yield of the test oil palm plant, and the first SNP marker is associated, after stratification and kinship correction, with the high-oil-production trait with a genome-wide −log₁₀(p-value) of at least 4.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 4.0 in the population.

Also, in some examples the population of oil palm plants comprises a Banting dura×AVROS pisifera population, the first QTL corresponds to one of QTL regions 1, 3, 4, 5, 6, 9, 10, 11, 12, 21, 26, 27, 28, 29, 30, 31, 32, 34, 35, 36, 37, 38, 40, 41, 42, 43, 44, 47, 49, 50, 51, 53, 55, or 56, and step (iii) further comprises applying a genotype model, thereby predicting the palm oil yield of the test oil palm plant.

Also, in some examples the population of oil palm plants comprises a Banting dura×AVROS pisifera population, the first QTL corresponds to one of QTL regions 17, 20, 49, or 55, and step (iii) further comprises applying a dominant model, thereby predicting the palm oil yield of the test oil palm plant.

Also, in some examples the population of oil palm plants comprises a Banting dura×AVROS pisifera population, the first QTL corresponds to one of QTL regions 2, 5, 9, 10, 15, 17, 24, 26, 27, 28, 29, 31, 32, 34, 35, 36, 39, 41, 44, 46, 47, 48, 50, 51, 56, or 57, and step (iii) further comprises applying a recessive model, thereby predicting the palm oil yield of the test oil palm plant.

As noted above, crossing dura and pisifera gives rise to palms with a third fruit type, the tenera. As also noted, tenera are typically used as commercial planting materials. Accordingly, in some examples the test oil palm plant is a tenera candidate agricultural production plant. In some examples the population of oil palm plants comprises an Ulu Remis dura×AVROS pisifera population, and the test oil palm plant is a tenera candidate agricultural production plant. Also, in some examples the population of oil palm plants comprises a Banting dura×AVROS pisifera population, and the test oil palm plant is a tenera candidate agricultural production plant.

As also noted above, oil palm breeding is primarily aimed at selecting for improved parental dura and pisifera breeding stock palms for production of superior tenera commercial planting materials. As also noted, parental dura breeding populations are generated by crossing among selected dura palms, whereas pisifera palms are normally female sterile and thus breeding populations thereof must be generated by crossing among selected teneras or by crossing selected teneras with selected pisiferas. Accordingly, in some examples the test oil palm plant is a plant for mother palm selection and propagation, a plant for introgressed mother palm selection and propagation, or a plant for pollen donor selection and propagation. In some examples, the population of oil palm plants comprises an Ulu Remis dura×Ulu Remis dura population, and the test oil palm plant is a plant for mother palm selection and propagation. Also in some examples, the population of oil palm plants comprises Ulu Remis dura×Ulu Remis dura population, and the test oil palm plant is a plant for introgressed mother palm selection and propagation. Also in some examples, the population of oil palm plants comprises an Ulu Remis dura×Banting dura population, and the test oil palm plant is a plant for mother palm selection and propagation. Also in some examples, the population of oil palm plants comprises a Banting dura×Banting dura population, and the test oil palm plant is a plant for mother palm selection and propagation. Also in some examples, the population of oil palm plants comprises a Banting dura×Banting dura population, and the test oil palm plant is a plant for introgressed mother palm selection and propagation. Also in some examples, the population of oil palm plants comprises an AVROS pisifera×AVROS tenera population, and the test oil palm plant is a plant for pollen donor selection and propagation. Also in some examples, the population of oil palm plants comprises an AVROS tenera×AVROS tenera population, and the test oil palm plant is a plant for pollen donor selection and propagation.

The method for predicting palm oil yield of a test oil palm plant also can be carried out by determining additional SNP genotypes, comparing the additional SNP genotypes to corresponding reference genotypes indicative of the high-oil-production trait, and further predicting palm oil yield of the test oil palm plant based on the extent to which the additional SNP genotypes match the corresponding reference SNP genotypes. This is because each SNP genotype can reflect a high-yield variant allele that contributes to a high-oil-production trait additively and/or synergistically with respect to the others.

Accordingly, in some examples step (i) further comprises determining, from the sample of the test oil palm plant, at least a second SNP genotype of the test oil palm plant, the second SNP genotype corresponding to a second SNP marker, the second SNP marker (a) being located in a second QTL for the high-oil-production trait and (b) being associated, after stratification and kinship correction, with the high-oil-production trait with a genome-wide −log₁₀(p-value) of at least 3.0 in the population or having a linkage disequilibrium r² value of at least 0.2 with respect to a second other SNP marker that is linked thereto and associated, after stratification and kinship correction, with the high-oil-production trait with a genome-wide −log₁₀(p-value) of at least 3.0 in the population. Moreover, in these examples step (ii) further comprises comparing the second SNP genotype of the test oil palm plant to a corresponding second reference SNP genotype indicative of the high-oil-production trait in the same genetic background as the population. In addition, in these examples the second QTL corresponds to one of QTL regions 1 to 57, with the proviso that the first QTL and the second QTL correspond to different QTL regions. In some of these examples, step (iii) further comprises predicting palm oil yield of the test oil palm plant based on the extent to which the second SNP genotype of the test oil palm plant matches the corresponding second reference SNP genotype.

Also in some examples, step (i) further comprises determining, from the sample of the test oil palm plant, at least a third SNP genotype to a fifty-seventh SNP genotype of the test oil palm plant, the third SNP genotype to the fifty-seventh SNP genotype corresponding to a third SNP marker to a fifty-seventh SNP marker, respectively, the third SNP marker to the fifty-seventh SNP marker (a) being located in a third QTL to a fifty-seventh QTL, respectively, for the high-oil-production trait and (b) being associated, after stratification and kinship correction, with the high-oil-production trait with a genome-wide −log₁₀(p-value) of at least 3.0 in the population or having linkage disequilibrium r² values of at least 0.2 with respect to a third other SNP marker to a fifty-seventh other SNP marker, respectively, that are linked thereto and associated, after stratification and kinship correction, with the high-oil-production trait with a genome-wide −log₁₀(p-value) of at least 3.0 in the population. Moreover, in these examples step (ii) further comprises comparing the third SNP genotype to the fifty-seventh SNP genotype of the test oil palm plant to a corresponding third reference SNP genotype to a corresponding fifty-seventh reference SNP genotype, respectively, indicative of the high-oil-production trait in the same genetic background as the population. In addition, in these examples the third QTL to the fifty-seventh QTL each correspond to one of QTL regions 1 to 57, with the proviso that the first QTL to the fifty-seventh QTL each correspond to different QTL regions. In some of these examples, step (iii) further comprises predicting palm oil yield of the test oil palm plant based on the extent to which the third SNP genotype to the fifty-seventh SNP genotype of the test oil palm plant match the corresponding third reference SNP genotype to the corresponding fifty-seventh reference SNP genotype, respectively.

Also provided is a method of selecting a high-palm-oil-yielding oil palm plant for agricultural production of palm oil. The method comprises a step of (a) predicting palm oil yield of a test oil palm plant. This step can be carried out according to the method described above, i.e. including a step of (i) determining, from a sample of a test oil palm plant of a population of oil palm plants, at least a first single nucleotide polymorphism (SNP) genotype of the test oil palm plant; a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first QTL is a region of the oil palm genome corresponding to one of QTL regions 1 to 57, as described above. The method also comprises a step of (b) field planting the test oil palm plant for agricultural production of palm oil if the palm oil yield of the test oil palm plant is predicted to be higher than average for the population based on step (a).

Also provided is a method of selecting a high-palm-oil-yielding oil palm plant for cultivation in cell culture. The method comprises a step of (a) predicting palm oil yield of a test oil palm plant. Again, this step can be carried out according to the method described above, i.e. including a step of (i) determining, from a sample of a test oil palm plant of a population of oil palm plants, at least a first single nucleotide polymorphism (SNP) genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first QTL is a region of the oil palm genome corresponding to one of QTL regions 1 to 57, as described above. The method also comprises a step of (b) subjecting at least one cell of the test oil palm plant to cultivation in cell culture if the palm oil yield of the test oil palm plant is predicted to be higher than average for the population based on step (a).

Also provided is a method of selecting a parental oil palm plant for use in breeding to obtain agricultural production plants or improved parental oil palm plants. As noted above, oil palm breeders can use the method, as applied to a test oil palm plant that is a mother palm or a pollen donor, to determine possible SNP genotypes of progeny to be generated by crossing the test oil palm plant with another oil palm plant, and moreover can choose specific palms, i.e. the test oil palm plant and another specific oil palm plant that has been similarly characterized, to be crossed on this basis. The method comprises a step of (a) predicting palm oil yield of a test oil palm plant. Again, this step can be carried out according to the method described above, i.e. including a step of (i) determining, from a sample of a test oil palm plant of a population of oil palm plants, at least a first single nucleotide polymorphism (SNP) genotype of the test oil palm plant, a step of (ii) comparing the first SNP genotype of the test oil palm plant to a corresponding first reference SNP genotype indicative of the high-oil-production trait in the same genetic background as the population, and a step of (iii) predicting palm oil yield of the test oil palm plant based on the extent to which the first SNP genotype of the test oil palm plant matches the corresponding first reference SNP genotype, wherein the first QTL is a region of the oil palm genome corresponding to one of QTL regions 1 to 57, as described above. The method also comprises a step of (b) selecting the test oil palm plant for use in breeding if the palm oil yield of tenera progeny of the test oil palm plant is predicted to be higher than average for the population based on step (a).

The application also is drawn to another method for predicting palm oil yield of a test oil palm plant. The method comprises a step of (i) determining, from a sample of a test oil palm plant of a population of oil palm plants, at least a first single nucleotide polymorphism (SNP) genotype to a tenth SNP genotype of the test oil palm plant, as discussed above. Thus, the first SNP genotype to the tenth SNP genotype of the test oil palm plant correspond to the constitution of SNP alleles at particular loci, or positions, on each chromosome in which the loci occur in the genome of the test oil palm plant, as discussed above. Also, each SNP allele may be classified, for example, based on allele frequency, e.g. as a major allele (A) or a minor allele (a). Thus, for example, each of the first SNP genotype to the tenth SNP genotype can correspond to two major alleles (A/A), one major allele and one minor allele (A/a), or two minor alleles (a/a), respectively.

The test oil palm plant can be an oil palm plant in any suitable form, as discussed above. For example, the test oil palm plant can be a seed, a seedling, a nursery phase plant, an immature phase plant, a cell culture plant, a zygotic embryo culture plant, or a somatic tissue culture plant. Also for example, the test oil palm plant can be a production phase plant, a mature palm, a mature mother palm, or a mature pollen donor.

In some examples, the population of oil palm plants comprises an Ulu Remis dura×AVROS pisifera population, a Banting dura×AVROS pisifera population, or a combination thereof. Also, in some examples the population of oil palm plants comprises an Ulu Remis dura×Ulu Remis dura, an Ulu Remis dura×Banting dura, a Banting dura×Banting dura, an AVROS pisifera×AVROS tenera population, an AVROS tenera×AVROS tenera population, or a combination thereof.

The first SNP genotype to the tenth SNP genotype corresponds to a first SNP marker to a tenth SNP marker, respectively, as discussed above. The first SNP marker to the tenth SNP marker are located in a first quantitative trait locus (QTL) to a tenth QTL, respectively, for a high-oil-production trait, as discussed above. The high-oil-production trait can comprise increased oil per palm plant, as discussed above.

The first SNP marker to the tenth SNP marker also are associated, after stratification and kinship correction, with the high-oil-production trait with a genome-wide −log₁₀(p-value) of at least 3.0 in the population or have a linkage disequilibrium r² value of at least 0.2 with respect to a first other SNP marker to a tenth other SNP marker, respectively, that are linked thereto and associated, after stratification and kinship correction, with the high-oil-production trait with a genome-wide −log₁₀(p-value) of at least 3.0 in the population, as discussed above. Accordingly, in some examples the first SNP marker to the tenth SNP marker are associated, after stratification and kinship correction, with the high-oil-production trait with a genome-wide −log₁₀(p-value) of at least 3.0 in the population. Also, in some examples the first SNP marker to the tenth SNP marker have a linkage disequilibrium r² value of at least 0.2 with respect to a first other SNP marker to a tenth other SNP marker, respectively, that are linked thereto and associated, after stratification and kinship correction, with the high-oil-production trait with a genome-wide −log₁₀(p-value) of at least 3.0 in the population. Also, in some examples a combination of each applies.

The method also comprises a step of (ii) comparing the first SNP genotype to the tenth SNP genotype of the test oil palm plant to a corresponding first reference SNP genotype to a corresponding tenth reference SNP genotype, respectively, indicative of the high-oil-production trait in the same genetic background as the population. The genetic background that is the same as the population can correspond, for example, to a population based on crossing oil palm plants of the same types as used to generate the population from which the test oil palm plant is sampled, e.g. an Ulu Remis dura×AVROS pisifera population, a Banting dura×AVROS pisifera population, or a combination thereof, or an Ulu Remis dura×Ulu Remis dura population, an Ulu Remis dura×Banting dura population, a Banting dura×Banting dura population, an AVROS pisifera×AVROS tenera population, an AVROS tenera×AVROS tenera population, or a combination thereof. The genetic background that is the same as the population also can correspond, for example, to a population based on crossing the same individual oil palm plants used to generate the population from which the test oil palm plant is sampled. The genetic background that is the same as the population also can correspond, for example, to the same actual population from which the test oil palm plant is sampled.

The method also comprises a step of (iii) predicting palm oil yield of the test oil palm plant based on the extent to which the first SNP genotype to the tenth SNP genotype of the test oil palm plant matches the corresponding first reference SNP genotype to the corresponding tenth reference SNP genotype, respectively, as discussed above. Thus, for example, the first SNP genotype to the tenth SNP genotype of the test oil palm plant can match the corresponding first reference SNP genotype to the corresponding tenth reference SNP genotype, respectively, based on both SNP genotypes of each pair sharing at least a first SNP allele indicative of the high-oil-production trait in the same genetic background as the population, as discussed above. Thus, for example, in some examples the first SNP genotype and the first reference SNP genotype are heterozygous for the first allele indicative of the high-oil production trait, i.e. both have only one copy of the SNP allele. Also, in some examples the first SNP genotype and the first reference SNP genotype are homozygous for the first allele indicative of the high-oil production trait, i.e. both have two copies of the SNP allele. Also, in some examples the first SNP genotype is heterozygous for the first allele indicative of the high-oil production trait and the first reference SNP genotype is homozygous for the first allele indicative of the high-oil production trait. Also, in some examples the first SNP genotype is homozygous for the first allele indicative of the high-oil production trait and the first reference SNP genotype is heterozygous for the first allele indicative of the high-oil production trait.

The step of predicting palm oil yield of the test oil palm plant can further comprise applying a model, such as a genotype model, a dominant model, or a recessive model, among others, in order to facilitate the predicting, as discussed above.

The first SNP marker to the tenth SNP marker can be ordered based on genomic selection, such that the first SNP marker to the tenth SNP marker provide the greatest predictive power of SNPs identified within the population.

For example, in accordance with some embodiments the population of oil palm plants comprises an Ulu Remis dura×AVROS pisifera population, the first SNP marker is located at nucleotide 31082003 of chromosome 1, the second SNP marker is located at nucleotide 31064632 of chromosome 1, the third SNP marker is located at nucleotide 50703308 of chromosome 2, the fourth SNP marker is located at nucleotide 31114410 of chromosome 1, the fifth SNP marker is located at nucleotide 31085464 of chromosome 1, the sixth SNP marker is located at nucleotide 29991680 of chromosome 1, the seventh SNP marker is located at nucleotide 23863567 of chromosome 15, the eighth SNP marker is located at nucleotide 23972701 of chromosome 15, the ninth SNP marker is located at nucleotide 31044765 of chromosome 1, the tenth SNP marker is located at nucleotide 23993289 of chromosome 15, and step (iii) further comprises applying a genotype model, thereby predicting the palm oil yield of the test oil palm plant.

Also, in accordance with some embodiments the population of oil palm plants comprises a Banting dura×AVROS pisifera population, the first SNP marker is located at nucleotide 28853893 of chromosome 9, the second SNP marker is located at nucleotide 2331299 of chromosome 13, the third SNP marker is located at nucleotide 1390286 of chromosome 7, the fourth SNP marker is located at nucleotide 32838961 of chromosome 9, the fifth SNP marker is located at nucleotide 26066534 of chromosome 1, the sixth SNP marker is located at nucleotide 5635482 of chromosome 16, the seventh SNP marker is located at nucleotide 18085183 of chromosome 6, the eighth SNP marker is located at nucleotide 28139147 of chromosome 1, the ninth SNP marker is located at nucleotide 26560042 of chromosome 6, the tenth SNP marker is located at nucleotide 18209857 of chromosome 6, and step (iii) further comprises applying a genotype model, thereby predicting the palm oil yield of the test oil palm plant.

The method can also be carried out by determining additional SNP genotypes, e.g. in order to improve prediction accuracy and/or to achieve maximum prediction accuracy. Thus, in some examples, step (i) further comprises determining, from the sample of the test oil palm plant, at least an eleventh SNP genotype to a thirtieth SNP genotype of the test oil palm plant, the eleventh SNP genotype to the thirtieth SNP genotype corresponding to an eleventh SNP marker to a thirtieth SNP marker, respectively, the eleventh SNP marker to the thirtieth SNP marker (a) being located in an eleventh QTL to a thirtieth QTL, respectively, for the high-oil-production trait and (b) being associated, after stratification and kinship correction, with the high-oil-production trait with a genome-wide −log₁₀(p-value) of at least 3.0 in the population or having linkage disequilibrium r² values of at least 0.2 with respect to an eleventh other SNP marker to a thirtieth other SNP marker, respectively, that are linked thereto and associated, after stratification and kinship correction, with the high-oil-production trait with a genome-wide −log₁₀(p-value) of at least 3.0 in the population. In accordance with these examples, step (ii) further comprises comparing the eleventh SNP genotype to the thirtieth SNP genotype of the test oil palm plant to a corresponding eleventh reference SNP genotype to a corresponding thirtieth reference SNP genotype, respectively, indicative of the high-oil-production trait in the same genetic background as the population. Also in accordance with these examples, step (iii) further comprises predicting palm oil yield of the test oil palm plant based on the extent to which the eleventh SNP genotype to the thirtieth SNP genotype of the test oil palm plant match the corresponding eleventh reference SNP genotype to the corresponding thirtieth reference SNP genotype, respectively. This approach can be used to improve prediction accuracy. Similarly, in some examples the method comprises determining and comparing even more SNP genotypes, e.g. a thirty-first SNP genotype to, for example, a fiftieth SNP genotype, a one-hundredth SNP genotype, a two-hundredth SNP genotype, a three-hundredth SNP genotype, a four-hundredth SNP genotype, a five-hundredth SNP genotype, or a one-thousandth SNP genotype. This approach can be used to further improve prediction accuracy and/or to achieve maximum prediction accuracy.

In some examples, the test oil palm plant is a tenera candidate agricultural production plant, as discussed above. Also, in some examples, the test oil palm plant is a plant for mother palm selection and propagation, a plant for introgressed mother palm selection and propagation, or a plant for pollen donor selection and propagation, as discussed above.

Also provided is a method of selecting a high-palm-oil-yielding oil palm plant for agricultural production of palm oil. The method comprises a step of (a) predicting palm oil yield of a test oil palm plant. This step can be carried out as discussed above. The method also comprises a step of (b) field planting the test oil palm plant for agricultural production of palm oil if the palm oil yield of the test oil palm plant is predicted to be higher than average for the population based on step (a).

Also provided is a method of selecting a high-palm-oil-yielding oil palm plant for cultivation in cell culture. The method comprises a step of (a) predicting palm oil yield of a test oil palm plant. This step can be carried out as discussed above. The method also includes a step of (b) subjecting at least one cell of the test oil palm plant to cultivation in cell culture if the palm oil yield of the test oil palm plant is predicted to be higher than average for the population based on step (a).

Also provided is a method of selecting a parental oil palm plant for use in breeding to obtain agricultural production plants or improved parental oil palm plants. The method comprises a step of (a) predicting palm oil yield of a test oil palm plant. This step can be carried out as discussed above. The method also includes a step of (b) selecting the test oil palm plant for use in breeding if the palm oil yield of tenera progeny of the test oil palm plant is predicted to be higher than average for the population based on step (a).

The following examples are for purposes of illustration and are not intended to limit the scope of the claims.

### Example

### Sampling and DNA Preparation

The sampling was conducted on genome-wide association study (also termed GWAS) mapping populations derived from an Ulu Remis dura×AVROS pisifera population (1,218 palms) and a Banting dura×AVROS pisifera population (953 palms). The sample selection was based on a good representation of oil per palm plant (also termed O/P) variants and pedigree recorded by the corresponding breeders. Total genomic DNA was isolated from unopened spear leaves using the DNAeasy® Plant Mini Kit (Qiagen, Limburg, Netherlands).

### Whole-Genome Re-Sequencing

The samples were pooled based on an equal molar concentration of DNA from each sample to form the sequencing DNA pool. A library was prepared for re-sequencing using HiSeq 2000 ™ sequencing systems (Illumina, San Diego, Calif.) to generate 100-bp pair-end reads to a 35× genome coverage, resulting in 1,015,758,056 raw reads. The pair-end reads were trimmed, filtered, and aligned to the published oil palm genome, as described by Singh et al., Nature 500:335-339 (2013), using BWA Mapper, as published by Li & Durbin, Bioinformatics 26:589-595 (2010), with default parameters. A total of approximately 6,846,197 putative SNPs were then called and filtered using SAMtools, as published by Li et al., Bioinformatics 25:2078-2079 (2009), with parameters of minimal mapping quality score of the SNP being 25, minimal depth 3×, and minimal SNP distance from a gap of 2 bp. Of the putative SNPs, 1,085,204 SNPs that were generated from Elaeis oleifera were removed. Also removed were 746,092 SNPs based on coverage (minimal 17 or maximal 53), genotype quality with minimal score of 8, and/or minor allele frequency (also termed “MAF”)<0.05. The other filtering steps were performed to remove 5,274,000 SNPs based on technical requirement of Illumina, including removal of pairs of SNPs with distance less than 60 bp and ambiguous nucleotides. This yielded 664,136 quality SNPs. According to linkage disequilibrium, r² cutoff being set at 0.3, a total of 200K SNPs with an average density of one SNP per 11 Kb were submitted to Illumina for design score calculation using Illumina's Assay Design Tool for Infinium (Illumina).

### SNP Genotyping

An OP100K Infinium array (Illumina) was used to assay the GWAS mapping populations (˜250 ng DNA/sample). The overnight amplified DNA samples were then fragmented by a controlled enzymatic process that did not require gel electrophoresis. The re-suspended DNA samples were hybridized to BeadChips (Illumina) after an overnight incubation in a corresponding capillary flow-through chamber. Allele specific hybridizations were fluorescently labeled and detected by a BeadArray Reader (Illumina). The raw reads were then analyzed using GenomeStudio Data Analysis software (Illumina) for automated genotyping calling and quality control. To generate the genotypic dataset for GWAS, only the SNPs that had minor allele frequency >0.01 and >90% of call rate were accepted. The missing genotype of those SNPs was subsequently imputed based on the mean of each marker, in accordance with Endelman, Plant Genome 4:250-255 (2011).

### Genetic Stratification and Population Analyses

The individuals in the study were first split into different populations based on their respective backgrounds, which addressed population structure effect. Within each population, kinship correction was carried out using relationship matrix between the individuals, which addressed cryptic relatedness.

### Phenotypic Data Compilation and GWAS

O/P corresponds to total yield of palm oil from total bunches harvested per oil palm plant per year. O/P is measured as FFB×O/B. FFB corresponds to total weight of bunches produced per palm per year. Measurement of FFB is typically conducted in the field during bunch harvesting. O/B corresponds to oil content per bunch. Measurement of O/B is carried out according to industry practice, as described by Blaak et al., “Methods of bunch analysis,” Breeding and Inheritance in the Oil Palm (Elaeis guineensis Jacq.) Part II, Vol. 4:146-155 (J. W. Afr. Ins. Oil Palm Res., 1963), with modifications as described by Rao et al., “A Critical Reexamination of the Method of Bunch Analysis in Oil Palm Breeding,” Palm Oil Research Institute Malaysia Occ Paper 9:1-28 (1983). Association analyses were conducted on 1,218 Ulu Remis dura×AVROS pisifera palms and 953 Banting dura×AVROS pisifera palms, respectively, based on a compressed mixed linear model with P3D analysis according to Zhang et al., Nature Genetics 42:355-360 (2010), in the rrBLUP program, in accordance with Endelman (2011). The total number of common SNPs was 48,784 SNPs with minor allele frequency >0.01. Genetic sub-structure resulting from cryptic relatedness was accounted for by including kinship matrix, in accordance with VanRaden, Journal of Dairy Science 91:4414-4423 (2008), as a random effect in the compressed mixed linear model method. The whole-genome significance −log₁₀(p-value) cutoff was fixed at ≥4 and 3 for the population of Ulu Remis dura×AVROS pisifera palms and Banting dura×AVROS pisifera palms, due to complexity nature of the O/P trait. The quantile-quantile (Q-Q) plots and Manhattan plots were then constructed using an R package qqman, in accordance with Turner, qqman: An R package for visualizing GWAS results using Q-Q and Manhattan plots, available at http://biorxiv.org/content/early/2014/05/14/005165 (last accessed Nov. 15, 2014). Inflated false-positive signals were also evaluated for both methods according to the genomic inflated factor (GIF) estimated in an R package GenABEL, in accordance with Aulchenko et al. (2007).

### SNP Effects and Statistical Analyses

The significant SNPs according to −log₁₀(p-value)≥3.0 were further analyzed for the genotype model-based SNP effects on O/P trait, illustrated in boxplots and followed by one-way ANOVA test with multi comparisons using R statistical program available at https://www.r-project.org/. The same analytical method was expanded to determine O/P association with the presence of one SNP allele, either a major allele (A) or a minor allele (a) through dominance model (A/A+A/a, a/a) and recessive model (A/A, A/a+a/a).

### Genomic Selection

For genomic selection, SNP markers were sorted based on their association score to the O/P trait. A total of 994 unique SNP markers were selected to define a range. Analyses were carried out with respect to SNP markers sorted based on their association score to the O/P trait, from high association to low association. Analyses also were carried out with respect to SNP markers in linkage disequilibrium with SNP markers sorted based on their association score to the O/P trait, from high association to low association. For the case of linkage disequilibrium, graphs were generated based on one random SNP per region of linkage disequilibrium, with a total of 100 iterations for marker selection and 50 cycles each for cross validations. A negative control also was carried out, by random selection of 500 SNP markers from among the SNP markers identified.

### Results

Oil production phenotype data for the Ulu Remis dura×AVROS pisifera population and the Banting dura×AVROS pisifera population, expressed as percentage O/P, are provided in TABLE 1. As can be seen, the Ulu Remis dura×AVROS pisifera population exhibited a mean O/P of 49.29 kg/palm/year, and the Banting dura×AVROS pisifera population exhibited a mean O/P of 45.1 kg/palm/year.

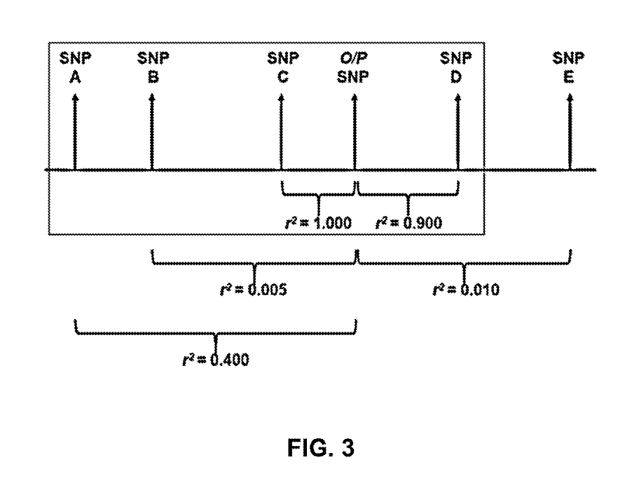

Fifty-seven QTL regions for the O/P trait in the Ulu Remis dura×AVROS pisifera population and the Banting dura×AVROS pisifera population were identified, as shown in TABLE 2, with elaboration in FIG. 3. The numbering of chromosomes and nucleotides thereof is in accordance with the 1.8 gigabase genome sequence of the African oil palm E. guineensis as described by Singh et al., Nature 500:335-339 (2013) and the supplementary information noted therein, as discussed above. The 57 QTL regions span 17,931,276 nucleotides, corresponding to approximately 0.9% of the oil palm genome.

One hundred and nineteen SNP markers that are informative with respect to O/P for the Ulu Remis dura×AVROS pisifera population and/or the Banting dura×AVROS pisifera population and that are located within the 57 QTLs were identified, as shown in TABLE 3, TABLE 4, TABLE 5, and TABLE 6. SNP identifying information and positional information is provided in TABLE 3. As can be seen in TABLE 4 and TABLE 5, each of the SNP markers yielded a genome-wide −log₁₀(p-value) of at least 3.0 in at least one of the Ulu Remis dura×AVROS pisifera population and/or the Banting dura×AVROS pisifera population with respect to at least one of a genotype model, a dominant model, or a recessive model. Indeed, many of the SNP markers yielded a genome-wide −log₁₀(p-value) of at least 3.0 in both populations and/or with respect to more than one of the models. Also, as can be seen in TABLE 6, for each of the SNP markers for which a minor SNP allele was detected in a given population, differences (termed δ) in mean percentage O/P for oil palm plants of the given population including a SNP allele associated with the high-oil-production trait (termed Max) versus oil palm plants of the given population lacking the SNP allele (termed Min), with respect to the genotype model in particular, ranged from 0.56% to 11.18% for the Ulu Remis dura×AVROS pisifera population and ranged from 0.18% to 33.53% for the Banting dura×AVROS pisifera population. Various SNP markers are informative with respect to both populations.

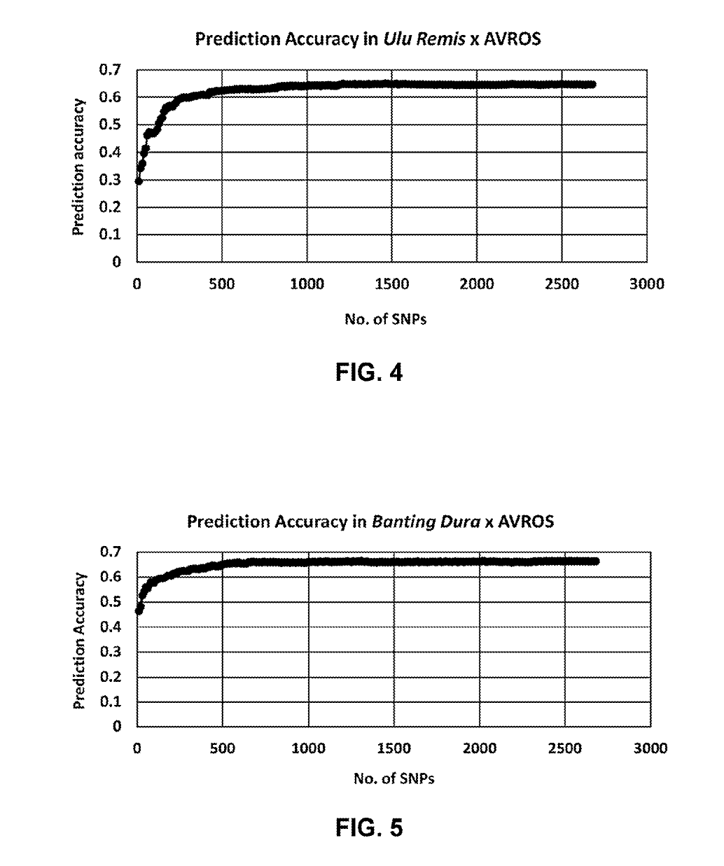

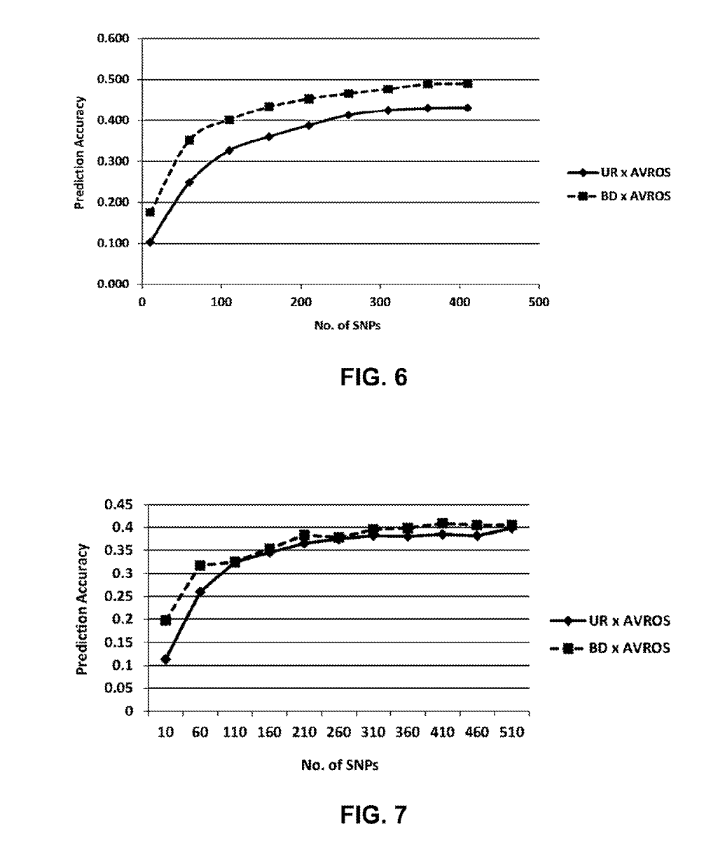

Regarding genomic selection, it was determined that approximately 500 SNP markers were needed in order to reach maximum prediction accuracy for both the Ulu Remis dura×AVROS pisifera population and the Banting dura×AVROS pisifera population, as shown in TABLE 7, FIG. 4, and FIG. 5. Regarding linkage disequilibrium, the results indicate that SNP markers in linkage disequilibrium with SNP markers sorted based on their association score to the O/P trait, from high association to low association, also can be used for prediction, as shown in TABLE 8 and FIG. 6. Regarding the negative control, results indicate a prediction accuracy of up to about 0.4 for both populations, as expected for use of randomly selected markers, as shown in TABLE 9 and FIG. 7.

TABLE 1
Oil per palm plant (also termed O/P), expressed as kg palm oil per
palm plant per year, for the Ulu Remis dura × AVROS pisifera population
and the Banting dura × AVROS pisifera population.

| | Mean | | | Median | Range |
|---|---|---|---|---|---|
| | kg/palm/ | St. | Coef. | kg/palm/ | kg/palm/ |
| Population | year | Dev. | Var. | year | year |
| Ulu Remis dura × | 49.29 | 11.93 | 0.24 | 48.9 | 67.1 |
| AVROS pisifera | | | | | |
| population | | | | | |
| Banting dura × | 45.1 | 10.28 | 0.23 | 45 | 66.1 |
| AVROS pisifera | | | | | |
| population | | | | | |

TABLE 2
QTL regions 1 to 57: Chromosome and
nucleotide position information.

| | | Start | Stop | |
|---|---|---|---|---|
| | | Nucleotide | Nucleotide | Length (bp) |
| QTL region | Chromosome | (bp) | (bp) | (Stop-Start + 1) |
| 1 | 1 | 18204491 | 18358401 | 153911 |
| 2 | 1 | 18922390 | 19167923 | 245534 |
| 3 | 1 | 19188077 | 19685080 | 497004 |
| 4 | 1 | 23276098 | 23456770 | 180673 |
| 5 | 1 | 26021716 | 26066534 | 44819 |
| 6 | 1 | 28110016 | 28234799 | 124784 |
| 7 | 1 | 29798161 | 30164329 | 366169 |
| 8 | 1 | 30684639 | 31160129 | 475491 |
| 9 | 1 | 37811723 | 38637229 | 825507 |
| 10 | 1 | 38659012 | 39206652 | 547641 |
| 11 | 1 | 39243858 | 39842157 | 598300 |
| 12 | 1 | 61305818 | 61572106 | 266289 |
| 13 | 2 | 1068379 | 1516571 | 448193 |
| 14 | 2 | 1616491 | 2016169 | 399679 |
| 15 | 2 | 17637996 | 17959911 | 321916 |
| 16 | 2 | 20732085 | 20977490 | 245406 |
| 17 | 2 | 31844836 | 31980071 | 135236 |
| 18 | 2 | 50449700 | 50857310 | 407611 |
| 19 | 2 | 50879601 | 51539414 | 659814 |
| 20 | 2 | 52821582 | 52960520 | 138939 |
| 21 | 3 | 42585292 | 42728875 | 143584 |
| 22 | 4 | 9561644 | 9701199 | 139556 |
| 23 | 4 | 12469969 | 13409114 | 939146 |
| 24 | 4 | 14672228 | 14789226 | 116999 |
| 25 | 5 | 395189 | 842107 | 446919 |
| 26 | 5 | 47205529 | 47293291 | 87763 |
| 27 | 5 | 48857594 | 48932286 | 74693 |
| 28 | 6 | 5943980 | 6002717 | 58738 |
| 29 | 6 | 6337822 | 6563232 | 225411 |
| 30 | 6 | 6818733 | 7281658 | 462926 |
| 31 | 6 | 17578027 | 18209857 | 631831 |
| 32 | 6 | 26204516 | 26755007 | 550492 |
| 33 | 6 | 36492757 | 36494757 | 2001 |
| 34 | 7 | 219790 | 1533149 | 1313360 |
| 35 | 8 | 8700733 | 9242332 | 541600 |
| 36 | 8 | 23767318 | 23957652 | 190335 |
| 37 | 8 | 26648547 | 26848102 | 199556 |
| 38 | 9 | 606020 | 1309231 | 703212 |
| 39 | 9 | 3499347 | 3638435 | 139089 |
| 40 | 9 | 28437588 | 28513671 | 76084 |
| 41 | 9 | 28581068 | 28912034 | 330967 |
| 42 | 9 | 32327318 | 32434321 | 107004 |
| 43 | 9 | 32538074 | 32540074 | 2001 |
| 44 | 9 | 32775289 | 33054696 | 279408 |
| 45 | 9 | 33133902 | 33254107 | 120206 |
| 46 | 10 | 15342814 | 15405953 | 63140 |
| 47 | 11 | 15933273 | 15943963 | 10691 |
| 48 | 12 | 12178551 | 12249693 | 71143 |
| 49 | 13 | 2052746 | 2447722 | 394977 |
| 50 | 13 | 14345084 | 14709650 | 364567 |
| 51 | 13 | 22031000 | 22147560 | 116561 |
| 52 | 15 | 23588504 | 24307350 | 718847 |
| 53 | 16 | 1511530 | 1596020 | 84491 |
| 54 | 16 | 2684531 | 2803682 | 119152 |
| 55 | 16 | 5535711 | 5995857 | 460147 |
| 56 | 16 | 8379248 | 8554851 | 175604 |
| 57 | 16 | 8883687 | 9269845 | 386159 |

TABLE 3
SNP markers in QTL regions 1 to 57: SNP identifying
information and positional information.

| | | QTL | | Position |
|---|---|---|---|---|
| SNP No. | SNP ID | region | Chromosome | (bp) |
| 1 | SD_SNP_000047088 | 1 | 1 | 18294251 |
| 2 | SD_SNP_000014031 | 2 | 1 | 19021805 |
| 3 | SD_SNP_000053381 | 3 | 1 | 19416298 |
| 4 | SD_SNP_000054224 | 4 | 1 | 23382088 |
| 5 | SD_SNP_000030351 | 5 | 1 | 26066534 |
| 6 | SD_SNP_000024794 | 6 | 1 | 28139147 |
| 7 | SD_SNP_000023238 | 7 | 1 | 29847596 |
| 8 | SD_SNP_000023244 | 7 | 1 | 29889833 |
| 9 | SD_SNP_000045974 | 7 | 1 | 29928222 |
| 10 | SD_SNP_000023235 | 7 | 1 | 29940135 |
| 11 | SD_SNP_000051735 | 7 | 1 | 29991680 |
| 12 | SD_SNP_000052219 | 7 | 1 | 29993615 |
| 13 | SD_SNP_000051520 | 7 | 1 | 30006599 |
| 14 | SD_SNP_000025118 | 8 | 1 | 30718952 |
| 15 | SD_SNP_000025117 | 8 | 1 | 30721069 |
| 16 | SD_SNP_000038788 | 8 | 1 | 31020182 |
| 17 | SD_SNP_000038785 | 8 | 1 | 31031067 |
| 18 | SD_SNP_000002587 | 8 | 1 | 31036295 |
| 19 | SD_SNP_000002585 | 8 | 1 | 31039673 |
| 20 | SD_SNP_000002584 | 8 | 1 | 31044765 |
| 21 | SD_SNP_000002583 | 8 | 1 | 31047554 |
| 22 | SD_SNP_000033086 | 8 | 1 | 31064632 |
| 23 | SD_SNP_000033087 | 8 | 1 | 31067895 |
| 24 | SD_SNP_000012440 | 8 | 1 | 31082003 |
| 25 | SD_SNP_000012441 | 8 | 1 | 31085464 |
| 26 | SD_SNP_000038089 | 8 | 1 | 31114410 |
| 27 | SD_SNP_000039597 | 8 | 1 | 31136302 |
| 28 | SD_SNP_000019139 | 9 | 1 | 38017787 |
| 29 | SD_SNP_000026478 | 10 | 1 | 38770530 |
| 30 | SD_SNP_000051396 | 11 | 1 | 39460825 |
| 31 | SD_SNP_000047235 | 11 | 1 | 39743362 |
| 32 | SD_SNP_000035566 | 12 | 1 | 61505339 |
| 33 | SD_SNP_000018354 | 13 | 2 | 1286832 |
| 34 | SD_SNP_000019443 | 13 | 2 | 1314457 |
| 35 | SD_SNP_000049161 | 14 | 2 | 1956976 |
| 36 | SD_SNP_000025928 | 14 | 2 | 1969929 |
| 37 | SD_SNP_000024448 | 15 | 2 | 17899789 |
| 38 | SD_SNP_000050442 | 16 | 2 | 20945042 |
| 39 | SD_SNP_000030830 | 17 | 2 | 31891554 |
| 40 | SD_SNP_000030831 | 17 | 2 | 31894732 |
| 41 | SD_SNP_000013826 | 18 | 2 | 50670957 |
| 42 | SD_SNP_000045345 | 18 | 2 | 50703308 |
| 43 | SD_SNP_000003849 | 18 | 2 | 50805429 |
| 44 | SD_SNP_000050168 | 19 | 2 | 51046557 |
| 45 | SD_SNP_000050167 | 19 | 2 | 51048301 |
| 46 | SD_SNP_000050166 | 19 | 2 | 51049777 |
| 47 | SD_SNP_000053948 | 19 | 2 | 51054672 |
| 48 | SD_SNP_000032720 | 20 | 2 | 52891093 |
| 49 | SD_SNP_000037233 | 21 | 3 | 42725437 |
| 50 | SD_SNP_000029835 | 22 | 4 | 9598633 |
| 51 | SD_SNP_000040840 | 23 | 4 | 12729488 |
| 52 | SD_SNP_000051461 | 23 | 4 | 13095539 |
| 53 | SD_SNP_000004705 | 24 | 4 | 14769521 |
| 54 | SD_SNP_000054827 | 25 | 5 | 760710 |
| 55 | SD_SNP_000007634 | 25 | 5 | 771100 |
| 56 | SD_SNP_000015048 | 26 | 5 | 47229487 |
| 57 | SD_SNP_000041456 | 27 | 5 | 48874130 |
| 58 | SD_SNP_000052151 | 28 | 6 | 5943980 |
| 59 | SD_SNP_000010796 | 28 | 6 | 5963880 |
| 60 | SD_SNP_000021121 | 28 | 6 | 5983589 |
| 61 | SD_SNP_000020929 | 29 | 6 | 6438250 |
| 62 | SD_SNP_000027651 | 30 | 6 | 6891275 |
| 63 | SD_SNP_000006840 | 31 | 6 | 17898768 |
| 64 | SD_SNP_000044394 | 31 | 6 | 18069342 |
| 65 | SD_SNP_000054985 | 31 | 6 | 18079752 |
| 66 | SD_SNP_000052370 | 31 | 6 | 18081983 |
| 67 | SD_SNP_000026677 | 31 | 6 | 18085183 |
| 68 | SD_SNP_000024441 | 31 | 6 | 18186584 |
| 69 | SD_SNP_000024439 | 31 | 6 | 18201224 |
| 70 | SD_SNP_000024437 | 31 | 6 | 18207393 |
| 71 | SD_SNP_000024436 | 31 | 6 | 18209857 |
| 72 | SD_SNP_000002684 | 32 | 6 | 26560042 |
| 73 | SD_SNP_000019392 | 33 | 6 | 36493757 |
| 74 | SD_SNP_000005015 | 34 | 7 | 935788 |
| 75 | SD_SNP_000030444 | 34 | 7 | 1136742 |
| 76 | SD_SNP_000053247 | 34 | 7 | 1155176 |
| 77 | SD_SNP_000013985 | 34 | 7 | 1379948 |
| 78 | SD_SNP_000013988 | 34 | 7 | 1390286 |
| 79 | SD_SNP_000025320 | 35 | 8 | 8866565 |
| 80 | SD_SNP_000016724 | 35 | 8 | 8946733 |
| 81 | SD_SNP_000018562 | 35 | 8 | 9049260 |
| 82 | SD_SNP_000031643 | 35 | 8 | 9104000 |
| 83 | SD_SNP_000031640 | 35 | 8 | 9116079 |
| 84 | SD_SNP_000009517 | 36 | 8 | 23834828 |
| 85 | SD_SNP_000021176 | 36 | 8 | 23908749 |
| 86 | SD_SNP_000051329 | 37 | 8 | 26648547 |
| 87 | SD_SNP_000033865 | 38 | 9 | 1194948 |
| 88 | SD_SNP_000041090 | 39 | 9 | 3556496 |
| 89 | SD_SNP_000009273 | 40 | 9 | 28449376 |
| 90 | SD_SNP_000003391 | 41 | 9 | 28583785 |
| 91 | SD_SNP_000048022 | 41 | 9 | 28853893 |
| 92 | SD_SNP_000025768 | 42 | 9 | 32347616 |
| 93 | SD_SNP_000054879 | 43 | 9 | 32539074 |
| 94 | SD_SNP_000041233 | 44 | 9 | 32838961 |
| 95 | SD_SNP_000052861 | 44 | 9 | 32950376 |
| 96 | SD_SNP_000038466 | 45 | 9 | 33192161 |
| 97 | SD_SNP_000021798 | 46 | 10 | 15357823 |
| 98 | SD_SNP_000016668 | 47 | 11 | 15933273 |
| 99 | SD_SNP_000001674 | 48 | 12 | 12189967 |
| 100 | SD_SNP_000049785 | 49 | 13 | 2331299 |
| 101 | SD_SNP_000026574 | 50 | 13 | 14571616 |
| 102 | SD_SNP_000031034 | 50 | 13 | 14637777 |
| 103 | SD_SNP_000045935 | 51 | 13 | 22081089 |
| 104 | SD_SNP_000047056 | 51 | 13 | 22146083 |
| 105 | SD_SNP_000040027 | 52 | 15 | 23863567 |
| 106 | SD_SNP_000026833 | 52 | 15 | 23968943 |
| 107 | SD_SNP_000026834 | 52 | 15 | 23972701 |
| 108 | SD_SNP_000026836 | 52 | 15 | 23981284 |
| 109 | SD_SNP_000026838 | 52 | 15 | 23993289 |
| 110 | SD_SNP_000026839 | 52 | 15 | 23995922 |
| 111 | SD_SNP_000031115 | 52 | 15 | 24139328 |
| 112 | SD_SNP_000015475 | 52 | 15 | 24267068 |
| 113 | SD_SNP_000028114 | 53 | 16 | 1596020 |
| 114 | SD_SNP_000010621 | 54 | 16 | 2771681 |
| 115 | SD_SNP_000033563 | 55 | 16 | 5618078 |
| 116 | SD_SNP_000033562 | 55 | 16 | 5620523 |
| 117 | SD_SNP_000033558 | 55 | 16 | 5635482 |
| 118 | SD_SNP_000032602 | 56 | 16 | 8438426 |
| 119 | SD_SNP_000039905 | 57 | 16 | 9042560 |

TABLE 4
SNP markers in QTL regions 1 to 57: Ulu Remis dura × AVROS
pisifera population major allele, minor allele, minor allele
frequency, and genome-wide −log₁₀(p-value) with respect
to a genotype model, a dominant model, and a recessive model.
SNP numbering is in accordance with Table 3.

| | Ulu Remis dura × AVROS pisifera | | | | | |
|---|---|---|---|---|---|---|
| | | | Minor | | | |
| SNP | Major | Minor | allele | [−log₁₀(p-value)] | | |
| No. | allele | allele | frequency | Genotype | Dominant | Recessive |
| 1 | G | A | 0.1942 | 0.786 | 0.000 | 0.582 |
| 2 | C | A | 0.2818 | 0.101 | 0.389 | 0.107 |
| 3 | G | A | 0.2444 | 0.691 | 0.000 | 0.551 |
| 4 | A | C | 0.1293 | 0.149 | 0.000 | 0.184 |
| 5 | A | G | 0.3062 | 0.164 | 0.144 | 0.314 |
| 6 | G | A | 0.2245 | 0.412 | 0.000 | 0.354 |
| 7 | G | A | 0.3777 | 4.176 | 0.000 | 3.549 |
| 8 | G | A | 0.2644 | 4.049 | 1.325 | 3.287 |
| 9 | A | G | 0.2642 | 4.084 | 1.325 | 3.432 |
| 10 | G | A | 0.2644 | 4.049 | 1.325 | 3.287 |
| 11 | A | G | 0.2648 | 4.577 | 2.375 | 2.907 |
| 12 | G | A | 0.3768 | 4.152 | 0.000 | 3.703 |
| 13 | A | C | 0.3771 | 4.195 | 0.000 | 3.703 |
| 14 | G | A | 0.1479 | 3.954 | 0.000 | 4.016 |
| 15 | A | G | 0.1434 | 4.437 | 0.000 | 4.760 |
| 16 | A | G | 0.3703 | 4.463 | 0.000 | 4.185 |
| 17 | A | G | 0.1671 | 2.798 | 0.000 | 4.050 |
| 18 | G | A | 0.3705 | 4.505 | 0.000 | 4.185 |
| 19 | A | G | 0.3703 | 4.463 | 0.000 | 4.185 |
| 20 | G | A | 0.3706 | 4.520 | 0.000 | 4.185 |
| 21 | A | G | 0.3702 | 4.449 | 0.000 | 4.185 |
| 22 | A | G | 0.2902 | 4.877 | 2.326 | 3.518 |
| 23 | A | G | 0.2221 | 3.674 | 1.963 | 4.739 |
| 24 | G | A | 0.4741 | 5.311 | 4.591 | 2.029 |
| 25 | A | G | 0.3729 | 4.646 | 0.000 | 4.185 |
| 26 | C | A | 0.372 | 4.807 | 0.000 | 4.185 |
| 27 | G | A | 0.3706 | 4.447 | 0.000 | 4.111 |
| 28 | A | G | 0.3387 | 0.961 | 0.000 | 0.907 |
| 29 | A | G | 0.3551 | 0.303 | 0.000 | 0.463 |
| 30 | G | A | 0.1881 | 0.790 | 0.000 | 0.580 |
| 31 | G | A | 0.1816 | 1.554 | 0.000 | 1.674 |
| 32 | G | A | 0.4762 | 0.528 | 0.926 | 0.160 |
| 33 | A | C | 0.3854 | 4.058 | 0.000 | 3.914 |
| 34 | A | G | 0.3851 | 4.127 | 0.000 | 4.097 |
| 35 | A | G | 0.2781 | 4.366 | 2.638 | 2.895 |
| 36 | G | A | 0.2798 | 4.270 | 2.638 | 2.833 |
| 37 | G | A | 0.107 | 0.089 | 0.000 | 0.046 |
| 38 | A | G | 0.3175 | 4.178 | 0.338 | 3.872 |
| 39 | G | A | 0.3888 | 0.341 | 0.097 | 0.284 |
| 40 | A | G | 0.2861 | 0.096 | 0.693 | 0.736 |
| 41 | A | G | 0.2344 | 4.226 | 0.000 | 4.234 |
| 42 | A | G | 0.2196 | 4.862 | 0.000 | 5.001 |
| 43 | A | G | 0.3599 | 4.036 | 0.567 | 4.505 |
| 44 | C | A | 0.3028 | 4.069 | 0.000 | 3.741 |
| 45 | G | A | 0.3028 | 4.112 | 0.000 | 3.741 |
| 46 | A | G | 0.298 | 4.016 | 0.000 | 3.893 |
| 47 | A | C | 0.3028 | 4.069 | 0.000 | 3.741 |
| 48 | G | A | 0.3144 | 0.678 | 0.000 | 0.621 |
| 49 | G | A | 0.4758 | 0.016 | 0.882 | 1.247 |
| 50 | G | A | 0.3392 | 2.800 | 0.000 | 4.692 |
| 51 | G | A | 0.3362 | 2.555 | 0.013 | 4.166 |
| 52 | A | G | 0.3251 | 2.523 | 0.020 | 4.078 |
| 53 | A | G | 0.2283 | 0.029 | 0.119 | 0.187 |
| 54 | G | A | 0.05629 | 4.061 | 0.000 | 3.915 |
| 55 | G | A | 0.05752 | 4.135 | 0.000 | 3.946 |
| 56 | A | G | 0.2999 | 0.381 | 0.000 | 0.241 |
| 57 | G | A | 0.4367 | 0.280 | 0.000 | 0.328 |
| 58 | C | A | 0.4232 | 0.193 | 0.168 | 0.801 |
| 59 | A | G | 0.4202 | 0.220 | 0.356 | 0.138 |
| 60 | A | G | 0.4638 | 0.957 | 0.499 | 0.833 |
| 61 | A | G | 0.2898 | 0.931 | 0.247 | 0.780 |
| 62 | A | G | 0.2914 | 0.097 | 0.532 | 0.979 |
| 63 | A | G | 0.009465 | 0.000 | 0.000 | 0.000 |
| 64 | G | A | 0.08703 | 0.185 | 0.000 | 0.153 |
| 65 | A | G | 0.08429 | 0.156 | 0.000 | 0.129 |
| 66 | C | A | 0.08463 | 0.142 | 0.000 | 0.080 |
| 67 | G | A | 0.08456 | 0.147 | 0.000 | 0.097 |
| 68 | A | G | 0.08792 | 0.005 | 0.000 | 0.068 |
| 69 | A | G | 0.08785 | 0.009 | 0.000 | 0.052 |
| 70 | C | A | 0.08622 | 0.011 | 0.000 | 0.001 |
| 71 | C | A | 0.08799 | 0.009 | 0.000 | 0.068 |
| 72 | C | A | 0.1127 | 0.294 | 0.000 | 0.345 |
| 73 | A | G | 0.4975 | 4.213 | 0.000 | 0.000 |
| 74 | A | C | 0.3324 | 1.078 | 0.776 | 0.869 |
| 75 | A | G | 0.3411 | 0.046 | 0.000 | 0.214 |
| 76 | C | A | 0.3396 | 0.027 | 0.000 | 0.197 |
| 77 | G | A | 0.3218 | 0.915 | 0.569 | 0.735 |
| 78 | A | G | 0.3303 | 0.608 | 0.448 | 0.523 |
| 79 | C | A | 0.2797 | 0.496 | 0.573 | 0.175 |
| 80 | G | A | 0.07677 | 0.333 | 0.000 | 0.324 |
| 81 | A | G | 0.4811 | 0.318 | 0.193 | 0.932 |
| 82 | G | A | 0.4117 | 0.108 | 0.000 | 0.318 |
| 83 | G | A | 0.4116 | 0.105 | 0.000 | 0.318 |
| 84 | A | G | 0.1602 | 0.038 | 0.000 | 0.070 |
| 85 | A | G | 0.226 | 0.534 | 0.000 | 0.694 |
| 86 | A | G | 0.3699 | 0.433 | 0.469 | 0.010 |
| 87 | G | A | 0.4975 | 1.157 | 0.565 | 0.522 |
| 88 | G | A | 0.07622 | 0.926 | 0.000 | 1.082 |
| 89 | A | G | 0.4035 | 0.107 | 0.038 | 0.258 |
| 90 | G | A | 0.2651 | 0.073 | 0.000 | 0.132 |
| 91 | G | A | 0.2486 | 0.023 | 0.000 | 0.032 |
| 92 | A | G | 0.424 | 0.566 | 0.806 | 0.121 |
| 93 | G | A | 0.2798 | 0.180 | 0.000 | 0.006 |
| 94 | G | A | 0.486 | 0.071 | 0.104 | 0.088 |
| 95 | G | A | 0.2463 | 0.018 | 0.000 | 0.037 |
| 96 | G | A | 0.08694 | 3.808 | 0.000 | 5.308 |
| 97 | G | A | 0.2516 | 0.691 | 0.041 | 0.955 |
| 98 | G | A | 0.01322 | 0.000 | 0.000 | 0.000 |
| 99 | A | G | 0.2227 | 0.479 | 0.000 | 0.367 |
| 100 | A | G | 0.2699 | 0.111 | 0.012 | 0.133 |
| 101 | G | A | 0.3258 | 0.089 | 0.000 | 0.225 |
| 102 | G | A | 0.3269 | 0.095 | 0.000 | 0.205 |
| 103 | G | A | 0.09252 | 0.044 | 0.000 | 0.013 |
| 104 | G | A | 0.09367 | 0.106 | 0.000 | 0.185 |
| 105 | A | G | 0.3265 | 4.527 | 2.439 | 3.206 |
| 106 | A | C | 0.3237 | 4.445 | 2.568 | 3.206 |
| 107 | C | A | 0.3235 | 4.520 | 2.805 | 3.137 |
| 108 | A | G | 0.3232 | 4.408 | 2.414 | 3.098 |
| 109 | C | A | 0.3248 | 4.509 | 2.805 | 2.990 |
| 110 | A | C | 0.325 | 4.327 | 2.568 | 3.057 |
| 111 | A | G | 0.3366 | 4.087 | 2.982 | 2.335 |
| 112 | G | A | 0.3377 | 4.255 | 3.230 | 2.393 |
| 113 | A | C | 0.02997 | 0.000 | 0.000 | 0.038 |
| 114 | G | A | 0.4376 | 4.244 | 2.827 | 2.149 |
| 115 | A | C | 0.3525 | 1.542 | 0.000 | 1.591 |
| 116 | G | A | 0.3528 | 1.514 | 0.000 | 1.478 |
| 117 | G | A | 0.3531 | 1.528 | 0.000 | 1.478 |
| 118 | A | G | 0.186 | 0.166 | 0.000 | 0.256 |
| 119 | G | A | 0.2685 | 0.218 | 0.470 | 0.020 |

TABLE 5
SNP markers in QTL regions 1 to 57: Banting dura × AVROS
pisifera population major allele, minor allele, minor allele
frequency, and genome-wide −log₁₀(p-value) with respect
to a genotype model, a dominant model, and a recessive model.
SNP numbering is in accordance with Table 3.

| | Banting dura × AVROS pisifera | | | | | |
|---|---|---|---|---|---|---|
| | | | Minor | | | |
| SNP | Major | Minor | allele | [−log₁₀(p-value)] | | |
| No. | allele | allele | frequency | Genotype | Dominant | Recessive |
| 1 | G | A | 0.2623 | 3.265 | 0.000 | 2.855 |
| 2 | C | A | 0.3739 | 2.654 | 0.595 | 3.251 |
| 3 | G | A | 0.2298 | 3.023 | 0.000 | 2.532 |
| 4 | A | C | 0.2874 | 3.116 | 1.343 | 2.277 |
| 5 | A | G | 0.3619 | 3.791 | 0.812 | 3.431 |
| 6 | G | A | 0.2986 | 3.623 | 0.000 | 2.987 |
| 7 | G | A | 0.3492 | 0.994 | 0.144 | 1.306 |
| 8 | G | A | 0.3229 | 0.354 | 0.179 | 0.388 |
| 9 | A | G | 0.3548 | 0.237 | 0.411 | 0.099 |
| 10 | G | A | 0.3248 | 0.060 | 0.146 | 0.080 |
| 11 | A | G | 0.3282 | 0.457 | 0.377 | 0.374 |
| 12 | G | A | 0.2987 | 0.827 | 0.350 | 0.728 |
| 13 | A | C | 0.2697 | 0.363 | 0.030 | 0.489 |
| 14 | G | A | 0.2994 | 0.125 | 0.374 | 0.049 |
| 15 | A | G | 0.2372 | 0.695 | 0.000 | 0.928 |
| 16 | A | G | 0.3076 | 0.697 | 0.049 | 0.992 |
| 17 | A | G | 0.2963 | 0.203 | 0.114 | 0.490 |
| 18 | G | A | 0.3013 | 0.803 | 0.081 | 1.029 |
| 19 | A | G | 0.3459 | 1.079 | 0.509 | 0.955 |
| 20 | G | A | 0.3059 | 0.976 | 0.064 | 1.293 |
| 21 | A | G | 0.3365 | 0.714 | 0.375 | 0.643 |
| 22 | A | G | 0.2995 | 0.136 | 0.493 | 0.026 |
| 23 | A | G | 0.3365 | 0.689 | 0.614 | 0.879 |
| 24 | G | A | 0.486 | 0.996 | 0.374 | 1.275 |
| 25 | A | G | 0.3534 | 0.917 | 0.982 | 0.501 |
| 26 | C | A | 0.3243 | 0.654 | 0.271 | 0.633 |
| 27 | G | A | 0.3012 | 0.637 | 0.002 | 0.858 |
| 28 | A | G | 0.3374 | 3.162 | 0.000 | 3.143 |
| 29 | A | G | 0.3404 | 3.445 | 0.000 | 3.243 |
| 30 | G | A | 0.15 | 3.089 | 0.000 | 2.924 |
| 31 | G | A | 0.1774 | 3.104 | 0.000 | 2.984 |
| 32 | A | G | 0.4897 | 3.456 | 2.348 | 2.018 |
| 33 | A | C | 0.2748 | 0.895 | 0.000 | 0.662 |
| 34 | A | G | 0.2787 | 1.036 | 0.000 | 0.797 |
| 35 | A | G | 0.3009 | 0.213 | 0.000 | 0.587 |
| 36 | G | A | 0.2641 | 0.193 | 0.000 | 0.432 |
| 37 | G | A | 0.2491 | 2.776 | 0.000 | 3.010 |
| 38 | A | G | 0.3444 | 0.325 | 0.435 | 0.384 |
| 39 | A | G | 0.4843 | 2.576 | 3.224 | 0.337 |
| 40 | A | G | 0.4543 | 2.096 | 0.056 | 3.390 |
| 41 | A | G | 0.3021 | 1.270 | 0.000 | 1.303 |
| 42 | A | G | 0.1542 | 1.258 | 0.000 | 1.264 |
| 43 | A | G | 0.3694 | 0.637 | 0.000 | 1.126 |
| 44 | C | A | 0.2658 | 0.010 | 0.000 | 0.103 |
| 45 | G | A | 0.2725 | 0.009 | 0.000 | 0.080 |
| 46 | A | G | 0.2429 | 0.114 | 0.000 | 0.208 |
| 47 | A | C | 0.2748 | 0.089 | 0.000 | 0.026 |
| 48 | G | A | 0.4652 | 2.710 | 3.105 | 0.942 |
| 49 | A | G | 0.465 | 3.064 | 2.714 | 0.812 |
| 50 | G | A | 0.135 | 0.045 | 0.000 | 0.094 |
| 51 | G | A | 0.4989 | 0.270 | 0.223 | 0.072 |
| 52 | A | G | 0.4494 | 0.048 | 0.207 | 0.090 |
| 53 | A | G | 0.1172 | 2.775 | 2.189 | 3.043 |
| 54 | G | A | 0.03162 | 0.000 | 0.000 | 0.096 |
| 55 | G | A | 0.03404 | 0.000 | 0.000 | 0.013 |
| 56 | A | G | 0.2519 | 3.246 | 0.000 | 3.351 |
| 57 | G | A | 0.3905 | 3.105 | 0.000 | 3.123 |
| 58 | A | C | 0.4693 | 3.112 | 0.595 | 3.973 |
| 59 | G | A | 0.4683 | 2.873 | 0.518 | 3.798 |
| 60 | A | G | 0.4401 | 3.393 | 0.525 | 3.706 |
| 61 | A | G | 0.2503 | 3.041 | 1.107 | 3.878 |
| 62 | A | G | 0.2056 | 3.408 | 0.000 | 2.918 |
| 63 | A | G | 0.06136 | 3.522 | 0.000 | 2.879 |
| 64 | G | A | 0.1656 | 3.298 | 0.000 | 4.769 |
| 65 | A | G | 0.1005 | 3.213 | 0.000 | 3.574 |
| 66 | C | A | 0.09914 | 2.982 | 0.000 | 3.299 |
| 67 | G | A | 0.1011 | 3.655 | 0.000 | 4.055 |
| 68 | A | G | 0.1368 | 3.082 | 0.000 | 4.459 |
| 69 | A | G | 0.1392 | 2.746 | 0.000 | 4.014 |
| 70 | C | A | 0.1278 | 2.612 | 0.000 | 3.679 |
| 71 | C | A | 0.1358 | 3.603 | 0.000 | 5.045 |
| 72 | C | A | 0.117 | 3.610 | 0.000 | 3.056 |
| 73 | A | G | 0.4297 | 1.141 | 0.000 | 1.170 |
| 74 | C | A | 0.4316 | 3.146 | 0.537 | 3.075 |
| 75 | A | G | 0.1294 | 3.023 | 0.000 | 3.303 |
| 76 | C | A | 0.1176 | 2.831 | 0.000 | 3.060 |
| 77 | A | G | 0.4745 | 3.033 | 0.859 | 2.665 |
| 78 | G | A | 0.4323 | 4.061 | 1.461 | 3.467 |
| 79 | C | A | 0.3158 | 3.109 | 0.708 | 2.867 |
| 80 | G | A | 0.1511 | 3.395 | 0.000 | 3.078 |
| 81 | A | G | 0.3742 | 3.446 | 1.352 | 2.500 |
| 82 | G | A | 0.3969 | 3.035 | 1.267 | 2.490 |
| 83 | G | A | 0.393 | 3.105 | 1.893 | 2.222 |
| 84 | A | G | 0.04615 | 2.824 | 0.000 | 3.011 |
| 85 | A | G | 0.1096 | 3.046 | 0.000 | 2.998 |
| 86 | G | A | 0.4871 | 3.230 | 2.054 | 1.424 |
| 87 | A | G | 0.3754 | 3.094 | 0.000 | 2.940 |
| 88 | G | A | 0.3428 | 2.449 | 1.303 | 3.167 |
| 89 | G | A | 0.4143 | 3.246 | 2.085 | 2.857 |
| 90 | G | A | 0.2958 | 3.082 | 0.000 | 1.986 |
| 91 | G | A | 0.3079 | 4.780 | 0.000 | 3.484 |
| 92 | G | A | 0.366 | 3.297 | 1.127 | 2.702 |
| 93 | G | A | 0.3243 | 3.514 | 0.000 | 2.799 |
| 94 | A | G | 0.4797 | 4.020 | 2.057 | 2.708 |
| 95 | G | A | 0.3013 | 3.288 | 0.000 | 3.326 |
| 96 | G | A | 0.2923 | 0.611 | 0.819 | 0.143 |
| 97 | G | A | 0.1317 | 2.188 | 0.000 | 3.691 |
| 98 | G | A | 0.07538 | 3.085 | 2.664 | 3.106 |
| 99 | A | G | 0.2421 | 2.213 | 0.000 | 3.363 |
| 100 | A | G | 0.4808 | 4.061 | 4.679 | 1.003 |
| 101 | G | A | 0.3077 | 3.114 | 0.000 | 3.197 |
| 102 | G | A | 0.2999 | 3.002 | 0.000 | 2.983 |
| 103 | G | A | 0.1898 | 3.015 | 0.000 | 3.094 |
| 104 | G | A | 0.1857 | 3.316 | 0.000 | 3.452 |
| 105 | A | G | 0.2288 | 0.416 | 0.000 | 0.005 |
| 106 | A | C | 0.2284 | 0.234 | 0.000 | 0.097 |
| 107 | C | A | 0.23 | 0.025 | 0.000 | 0.362 |
| 108 | A | G | 0.2286 | 0.140 | 0.000 | 0.203 |
| 109 | C | A | 0.2809 | 0.012 | 0.405 | 0.196 |
| 110 | A | C | 0.2781 | 0.007 | 0.459 | 0.201 |
| 111 | A | G | 0.2793 | 0.054 | 0.459 | 0.134 |
| 112 | G | A | 0.2753 | 0.175 | 0.497 | 0.505 |
| 113 | A | C | 0.07273 | 3.103 | 0.000 | 2.982 |
| 114 | A | G | 0.4569 | 0.372 | 0.055 | 0.512 |
| 115 | C | A | 0.3662 | 2.379 | 3.005 | 0.712 |
| 116 | A | G | 0.372 | 3.171 | 3.225 | 1.185 |
| 117 | A | G | 0.3766 | 3.711 | 3.404 | 1.562 |
| 118 | A | G | 0.3786 | 3.448 | 0.000 | 3.484 |
| 119 | G | A | 0.4838 | 2.233 | 0.125 | 3.937 |

TABLE 6
SNP markers in QTL regions 1 to 57: Differences (termed δ) in
mean percentage O/P for oil palm plants including a SNP allele
associated with the high-oil-production trait (termed Max) versus
oil palm plants lacking the SNP allele (termed Min), with respect
to the genotype model for the Ulu Remis dura × AVROS pisifera
population and the Banting dura × AVROS pisifera population.

| | SNP effects (Genotype): | | | SNP effects (Genotype): | | |
|---|---|---|---|---|---|---|
| | Ulu Remis dura × AVROS | | | Banting dura × AVROS | | |
| | pisifera mean O/P (%) | | | pisifera mean O/P (%) | | |
| SNP No. | Min | Max | δ | Min | Max | δ |
| 1 | n.s. | n.s. | n.s. | 36.760 | 46.720 | 9.960 |
| 2 | n.s. | n.s. | n.s. | n.s. | n.s. | n.s. |
| 3 | n.s. | n.s. | n.s. | 34.860 | 46.680 | 11.820 |
| 4 | n.s. | n.s. | n.s. | 38.870 | 46.710 | 7.840 |
| 5 | n.s. | n.s. | n.s. | 35.530 | 48.250 | 12.720 |
| 6 | n.s. | n.s. | n.s. | 13.100 | 46.630 | 33.530 |
| 7 | 48.290 | 52.090 | 3.810 | n.s. | n.s. | n.s. |
| 8 | 47.630 | 51.970 | 4.340 | n.s. | n.s. | n.s. |
| 9 | 47.630 | 51.970 | 4.340 | n.s. | n.s. | n.s. |
| 10 | 47.630 | 51.970 | 4.340 | n.s. | n.s. | n.s. |
| 11 | 47.880 | 51.580 | 3.700 | n.s. | n.s. | n.s. |
| 12 | 48.230 | 52.230 | 4.000 | n.s. | n.s. | n.s. |
| 13 | 48.230 | 52.240 | 4.010 | n.s. | n.s. | n.s. |
| 14 | n.s. | n.s. | n.s. | n.s. | n.s. | n.s. |
| 15 | 47.790 | 53.030 | 5.250 | n.s. | n.s. | n.s. |
| 16 | 48.120 | 52.500 | 4.380 | n.s. | n.s. | n.s. |
| 17 | n.s. | n.s. | n.s. | n.s. | n.s. | n.s. |
| 18 | 48.120 | 52.520 | 4.400 | n.s. | n.s. | n.s. |
| 19 | 48.120 | 52.500 | 4.380 | n.s. | n.s. | n.s. |
| 20 | 48.120 | 52.520 | 4.400 | n.s. | n.s. | n.s. |
| 21 | 48.120 | 52.500 | 4.380 | n.s. | n.s. | n.s. |
| 22 | 47.880 | 51.470 | 3.590 | n.s. | n.s. | n.s. |
| 23 | n.s. | n.s. | n.s. | n.s. | n.s. | n.s. |
| 24 | 48.390 | 50.570 | 2.190 | n.s. | n.s. | n.s. |
| 25 | 48.170 | 52.270 | 4.100 | n.s. | n.s. | n.s. |
| 26 | 48.130 | 52.470 | 4.340 | n.s. | n.s. | n.s. |
| 27 | 48.120 | 52.500 | 4.380 | n.s. | n.s. | n.s. |
| 28 | n.s. | n.s. | n.s. | 41.030 | 46.720 | 5.690 |
| 29 | n.s. | n.s. | n.s. | 41.240 | 46.880 | 5.640 |
| 30 | n.s. | n.s. | n.s. | 41.910 | 46.380 | 4.470 |
| 31 | n.s. | n.s. | n.s. | 41.410 | 47.000 | 5.590 |
| 32 | n.s. | n.s. | n.s. | 41.540 | 48.320 | 6.790 |
| 33 | 45.610 | 50.380 | 4.770 | n.s. | n.s. | n.s. |
| 34 | 45.570 | 50.400 | 4.830 | n.s. | n.s. | n.s. |
| 35 | 48.590 | 49.750 | 1.170 | n.s. | n.s. | n.s. |
| 36 | 48.610 | 49.740 | 1.130 | n.s. | n.s. | n.s. |
| 37 | n.s. | n.s. | n.s. | n.s. | n.s. | n.s. |
| 38 | 46.920 | 51.270 | 4.350 | n.s. | n.s. | n.s. |
| 39 | n.s. | n.s. | n.s. | n.s. | n.s. | n.s. |
| 40 | n.s. | n.s. | n.s. | n.s. | n.s. | n.s. |
| 41 | 47.280 | 50.990 | 3.720 | n.s. | n.s. | n.s. |
| 42 | 47.100 | 50.940 | 3.840 | n.s. | n.s. | n.s. |
| 43 | 47.940 | 50.730 | 2.780 | n.s. | n.s. | n.s. |
| 44 | 47.370 | 51.290 | 3.920 | n.s. | n.s. | n.s. |
| 45 | 47.370 | 51.290 | 3.920 | n.s. | n.s. | n.s. |
| 46 | 47.400 | 51.310 | 3.920 | n.s. | n.s. | n.s. |
| 47 | 47.370 | 51.290 | 3.920 | n.s. | n.s. | n.s. |
| 48 | n.s. | n.s. | n.s. | n.s. | n.s. | n.s. |
| 49 | n.s. | n.s. | n.s. | 43.300 | 47.640 | 4.340 |
| 50 | n.s. | n.s. | n.s. | n.s. | n.s. | n.s. |
| 51 | n.s. | n.s. | n.s. | n.s. | n.s. | n.s. |
| 52 | n.s. | n.s. | n.s. | n.s. | n.s. | n.s. |
| 53 | n.s. | n.s. | n.s. | n.s. | n.s. | n.s. |
| 54 | 45.230 | 49.800 | 4.570 | n.s. | n.s. | n.s. |
| 55 | 45.260 | 49.810 | 4.560 | n.s. | n.s. | n.s. |
| 56 | n.s. | n.s. | n.s. | 43.170 | 47.380 | 4.200 |
| 57 | n.s. | n.s. | n.s. | 40.100 | 46.430 | 6.330 |
| 58 | n.s. | n.s. | n.s. | 43.490 | 45.960 | 2.470 |
| 59 | n.s. | n.s. | n.s. | n.s. | n.s. | n.s. |
| 60 | n.s. | n.s. | n.s. | 43.470 | 45.950 | 2.480 |
| 61 | n.s. | n.s. | n.s. | 42.970 | 49.020 | 6.040 |
| 62 | n.s. | n.s. | n.s. | 43.100 | 48.420 | 5.320 |
| 63 | n.s. | n.s. | n.s. | 39.450 | 45.450 | 6.000 |
| 64 | n.s. | n.s. | n.s. | 41.680 | 46.810 | 5.130 |
| 65 | n.s. | n.s. | n.s. | 41.130 | 45.990 | 4.860 |
| 66 | n.s. | n.s. | n.s. | n.s. | n.s. | n.s. |
| 67 | n.s. | n.s. | n.s. | 40.830 | 46.090 | 5.260 |
| 68 | n.s. | n.s. | n.s. | 41.620 | 46.810 | 5.200 |
| 69 | n.s. | n.s. | n.s. | n.s. | n.s. | n.s. |
| 70 | n.s. | n.s. | n.s. | n.s. | n.s. | n.s. |
| 71 | n.s. | n.s. | n.s. | 41.790 | 46.810 | 5.020 |
| 72 | n.s. | n.s. | n.s. | 36.680 | 45.830 | 9.150 |
| 73 | 49.220 | 60.400 | 11.180 | n.s. | n.s. | n.s. |
| 74 | n.s. | n.s. | n.s. | 42.920 | 45.560 | 2.640 |
| 75 | n.s. | n.s. | n.s. | 41.730 | 46.210 | 4.470 |
| 76 | n.s. | n.s. | n.s. | n.s. | n.s. | n.s. |
| 77 | n.s. | n.s. | n.s. | 41.510 | 45.470 | 3.960 |
| 78 | n.s. | n.s. | n.s. | 39.840 | 45.490 | 5.640 |
| 79 | n.s. | n.s. | n.s. | 44.890 | 45.330 | 0.450 |
| 80 | n.s. | n.s. | n.s. | 37.340 | 45.780 | 8.440 |
| 81 | n.s. | n.s. | n.s. | 43.480 | 46.410 | 2.930 |
| 82 | n.s. | n.s. | n.s. | 40.770 | 45.860 | 5.090 |
| 83 | n.s. | n.s. | n.s. | 41.540 | 45.710 | 4.180 |
| 84 | n.s. | n.s. | n.s. | n.s. | n.s. | n.s. |
| 85 | n.s. | n.s. | n.s. | 42.100 | 45.910 | 3.820 |
| 86 | n.s. | n.s. | n.s. | 42.840 | 48.070 | 5.220 |
| 87 | n.s. | n.s. | n.s. | 43.860 | 45.480 | 1.620 |
| 88 | n.s. | n.s. | n.s. | n.s. | n.s. | n.s. |
| 89 | n.s. | n.s. | n.s. | 44.200 | 52.610 | 8.410 |
| 90 | n.s. | n.s. | n.s. | 37.290 | 46.480 | 9.190 |
| 91 | n.s. | n.s. | n.s. | 37.290 | 47.030 | 9.740 |
| 92 | n.s. | n.s. | n.s. | 43.530 | 46.140 | 2.610 |
| 93 | n.s. | n.s. | n.s. | 36.840 | 47.360 | 10.520 |
| 94 | n.s. | n.s. | n.s. | 41.390 | 48.770 | 7.370 |
| 95 | n.s. | n.s. | n.s. | 43.900 | 46.980 | 3.080 |
| 96 | n.s. | n.s. | n.s. | n.s. | n.s. | n.s. |
| 97 | n.s. | n.s. | n.s. | n.s. | n.s. | n.s. |
| 98 | n.s. | n.s. | n.s. | 40.080 | 45.250 | 5.170 |
| 99 | n.s. | n.s. | n.s. | n.s. | n.s. | n.s. |
| 100 | n.s. | n.s. | n.s. | 39.000 | 56.330 | 17.330 |
| 101 | n.s. | n.s. | n.s. | 41.950 | 46.290 | 4.340 |
| 102 | n.s. | n.s. | n.s. | 41.950 | 46.070 | 4.120 |
| 103 | n.s. | n.s. | n.s. | 45.020 | 45.200 | 0.180 |
| 104 | n.s. | n.s. | n.s. | 44.970 | 45.300 | 0.340 |
| 105 | 47.830 | 50.400 | 2.570 | n.s. | n.s. | n.s. |
| 106 | 47.910 | 50.320 | 2.410 | n.s. | n.s. | n.s. |
| 107 | 47.960 | 50.270 | 2.310 | n.s. | n.s. | n.s. |
| 108 | 47.910 | 50.320 | 2.410 | n.s. | n.s. | n.s. |
| 109 | 48.010 | 50.250 | 2.240 | n.s. | n.s. | n.s. |
| 110 | 47.950 | 50.300 | 2.360 | n.s. | n.s. | n.s. |
| 111 | 48.280 | 50.090 | 1.810 | n.s. | n.s. | n.s. |
| 112 | 48.240 | 50.120 | 1.870 | n.s. | n.s. | n.s. |
| 113 | n.s. | n.s. | n.s. | 30.800 | 45.440 | 14.640 |
| 114 | 48.860 | 49.420 | 0.560 | n.s. | n.s. | n.s. |
| 115 | n.s. | n.s. | n.s. | n.s. | n.s. | n.s. |
| 116 | n.s. | n.s. | n.s. | 42.210 | 45.500 | 3.290 |
| 117 | n.s. | n.s. | n.s. | 42.110 | 45.620 | 3.510 |
| 118 | n.s. | n.s. | n.s. | 42.310 | 45.920 | 3.610 |
| 119 | n.s. | n.s. | n.s. | n.s. | n.s. | n.s. |

SNP numbering is in accordance with Table 3.
Abbreviation “n.s.” means not statistically significant.

TABLE 7
Prediction accuracy of genomic selection based on SNP
markers sorted based on association score to O/P trait.

| | Ulu Remis dura × AVROS | | Banting dura × AVROS | |
|---|---|---|---|---|
| | pisifera population | | pisifera population | |
| | Predic- | Stan- | Predic- | Stan- |
| Number of | tion | dard | tion | dard |
| SNP markers | accuracy | deviation | accuracy | deviation |
| 10 | 0.296 | 0.034 | 0.463 | 0.034 |
| 20 | 0.343 | 0.034 | 0.483 | 0.034 |
| 30 | 0.358 | 0.033 | 0.526 | 0.033 |
| 40 | 0.395 | 0.032 | 0.539 | 0.032 |
| 50 | 0.415 | 0.031 | 0.558 | 0.031 |
| 60 | 0.463 | 0.027 | 0.554 | 0.029 |
| 70 | 0.473 | 0.027 | 0.567 | 0.029 |
| 80 | 0.471 | 0.027 | 0.579 | 0.029 |
| 90 | 0.469 | 0.027 | 0.576 | 0.028 |
| 100 | 0.469 | 0.027 | 0.576 | 0.028 |
| 110 | 0.475 | 0.027 | 0.588 | 0.027 |
| 120 | 0.484 | 0.027 | 0.590 | 0.027 |
| 130 | 0.505 | 0.026 | 0.593 | 0.027 |
| 140 | 0.518 | 0.025 | 0.594 | 0.027 |
| 150 | 0.527 | 0.025 | 0.594 | 0.027 |
| 160 | 0.549 | 0.025 | 0.596 | 0.027 |
| 170 | 0.561 | 0.025 | 0.601 | 0.027 |
| 180 | 0.560 | 0.024 | 0.606 | 0.027 |
| 190 | 0.569 | 0.024 | 0.606 | 0.027 |
| 200 | 0.569 | 0.024 | 0.604 | 0.027 |
| 210 | 0.566 | 0.024 | 0.612 | 0.027 |
| 220 | 0.576 | 0.024 | 0.615 | 0.027 |
| 230 | 0.580 | 0.024 | 0.613 | 0.027 |
| 240 | 0.590 | 0.024 | 0.620 | 0.026 |
| 250 | 0.593 | 0.024 | 0.622 | 0.026 |
| 260 | 0.596 | 0.024 | 0.623 | 0.026 |
| 270 | 0.599 | 0.024 | 0.625 | 0.026 |
| 280 | 0.600 | 0.025 | 0.623 | 0.026 |
| 290 | 0.599 | 0.025 | 0.624 | 0.025 |
| 300 | 0.599 | 0.025 | 0.624 | 0.025 |
| 310 | 0.599 | 0.023 | 0.631 | 0.025 |
| 320 | 0.602 | 0.023 | 0.632 | 0.025 |
| 330 | 0.603 | 0.023 | 0.633 | 0.025 |
| 340 | 0.607 | 0.023 | 0.633 | 0.025 |
| 350 | 0.606 | 0.023 | 0.632 | 0.025 |
| 360 | 0.607 | 0.023 | 0.632 | 0.025 |
| 370 | 0.608 | 0.023 | 0.634 | 0.025 |
| 380 | 0.609 | 0.023 | 0.635 | 0.025 |
| 390 | 0.610 | 0.023 | 0.635 | 0.025 |
| 400 | 0.608 | 0.023 | 0.635 | 0.025 |
| 410 | 0.609 | 0.023 | 0.639 | 0.025 |
| 420 | 0.608 | 0.023 | 0.641 | 0.025 |
| 430 | 0.619 | 0.023 | 0.641 | 0.025 |
| 440 | 0.618 | 0.023 | 0.645 | 0.025 |
| 450 | 0.621 | 0.023 | 0.644 | 0.025 |
| 460 | 0.620 | 0.022 | 0.643 | 0.025 |
| 470 | 0.624 | 0.022 | 0.642 | 0.025 |
| 480 | 0.623 | 0.022 | 0.645 | 0.025 |
| 490 | 0.624 | 0.022 | 0.644 | 0.025 |
| 500 | 0.625 | 0.022 | 0.651 | 0.025 |
| 510 | 0.625 | 0.022 | 0.651 | 0.025 |
| 520 | 0.627 | 0.022 | 0.652 | 0.025 |
| 530 | 0.627 | 0.022 | 0.654 | 0.025 |
| 540 | 0.627 | 0.022 | 0.654 | 0.025 |
| 550 | 0.628 | 0.022 | 0.654 | 0.025 |
| 560 | 0.628 | 0.022 | 0.656 | 0.025 |
| 570 | 0.627 | 0.022 | 0.656 | 0.025 |
| 580 | 0.630 | 0.022 | 0.655 | 0.025 |
| 590 | 0.629 | 0.022 | 0.658 | 0.025 |
| 600 | 0.628 | 0.022 | 0.656 | 0.025 |

TABLE 8
Prediction accuracy of genomic selection based on SNP
markers in linkage disequilibrium with SNP markers
sorted based on association score to O/P trait.

| | Ulu Remis dura × AVROS | | Banting dura × AVROS | |
|---|---|---|---|---|
| | pisifera population | | pisifera population | |
| | Predic- | Stan- | Predic- | Stan- |
| Number of | tion | dard | tion | dard |
| SNP markers | accuracy | deviation | accuracy | deviation |
| 10 | 0.104 | 0.037 | 0.176 | 0.040 |
| 60 | 0.249 | 0.039 | 0.352 | 0.028 |
| 110 | 0.326 | 0.028 | 0.402 | 0.031 |
| 160 | 0.361 | 0.030 | 0.433 | 0.030 |
| 210 | 0.388 | 0.028 | 0.453 | 0.029 |
| 260 | 0.414 | 0.025 | 0.465 | 0.030 |
| 310 | 0.425 | 0.031 | 0.477 | 0.032 |
| 360 | 0.430 | 0.034 | 0.489 | 0.038 |
| 410 | 0.431 | 0.032 | 0.490 | 0.038 |

TABLE 9
Prediction accuracy of genomic selection
based on randomly selected SNP markers.

| | Ulu Remis dura × AVROS | | Banting dura × AVROS | |
|---|---|---|---|---|
| | pisifera population | | pisifera population | |
| | Predic- | Stan- | Predic- | Stan- |
| Number of | tion | dard | tion | dard |
| SNP markers | accuracy | deviation | accuracy | deviation |
| 10 | 0.113 | 0.040 | 0.197 | 0.039 |
| 60 | 0.259 | 0.033 | 0.318 | 0.034 |
| 110 | 0.325 | 0.035 | 0.325 | 0.033 |
| 160 | 0.345 | 0.032 | 0.354 | 0.030 |
| 210 | 0.366 | 0.032 | 0.384 | 0.029 |
| 260 | 0.375 | 0.031 | 0.378 | 0.033 |
| 310 | 0.382 | 0.032 | 0.395 | 0.031 |
| 360 | 0.381 | 0.036 | 0.399 | 0.033 |
| 410 | 0.385 | 0.035 | 0.409 | 0.033 |
| 460 | 0.382 | 0.033 | 0.405 | 0.032 |
| 510 | 0.398 | 0.032 | 0.405 | 0.035 |

### INDUSTRIAL APPLICABILITY

The methods disclosed herein are useful for predicting oil yield of a test oil palm plant, and thus for improving commercial production of palm oil.

1. A method for predicting palm oil yield of a test oil palm plant, the method comprising the steps of:
(i) determining, from a sample of a test oil palm plant of a population of oil palm plants, at least a first single nucleotide polymorphism (SNP) genotype of the test oil palm plant, the first SNP genotype corresponding to a first SNP marker, the first SNP marker (a) being located in a first quantitative trait locus (QTL) for a high-oil-production trait and (b) being associated, after stratification and kinship correction, with the high-oil-production trait with a genome-wide −log₁₀ (p-value) of at least 3.0 in the population or having a linkage disequilibrium r² value of at least 0.2 with respect to a first other SNP marker that is linked thereto and associated, after stratification and kinship correction, with the high-oil-production trait with a genome-wide −log₁₀(p-value) of at least 3.0 in the population;
(ii) comparing the first SNP genotype of the test oil palm plant to a corresponding first reference SNP genotype indicative of the high-oil-production trait in the same genetic background as the population; and
(iii) predicting palm oil yield of the test oil palm plant based on the extent to which the first SNP genotype of the test oil palm plant matches the corresponding first reference SNP genotype,
wherein the first QTL is a region of the oil palm genome corresponding to one of:
(1) QTL region 1, extending from nucleotide 18204491 to 18358401 of chromosome 1;
(2) QTL region 2, extending from nucleotide 18922390 to 19167923 of chromosome 1;
(3) QTL region 3, extending from nucleotide 19188077 to 19685080 of chromosome 1;
(4) QTL region 4, extending from nucleotide 23276098 to 23456770 of chromosome 1;
(5) QTL region 5, extending from nucleotide 26021716 to 26066534 of chromosome 1;
(6) QTL region 6, extending from nucleotide 28110016 to 28234799 of chromosome 1;
(7) QTL region 7, extending from nucleotide 29798161 to 30164329 of chromosome 1;
(8) QTL region 8, extending from nucleotide 30684639 to 31160129 of chromosome 1;
(9) QTL region 9, extending from nucleotide 37811723 to 38637229 of chromosome 1;
(10) QTL region 10, extending from nucleotide 38659012 to 39206652 of chromosome 1;
(11) QTL region 11, extending from nucleotide 39243858 to 39842157 of chromosome 1;
(12) QTL region 12, extending from nucleotide 61305818 to 61572106 of chromosome 1;
(13) QTL region 13, extending from nucleotide 1068379 to 1516571 of chromosome 2;
(14) QTL region 14, extending from nucleotide 1616491 to 2016169 of chromosome 2;
(15) QTL region 15, extending from nucleotide 17637996 to 17959911 of chromosome 2;
(16) QTL region 16, extending from nucleotide 20732085 to 20977490 of chromosome 2;
(17) QTL region 17, extending from nucleotide 31844836 to 31980071 of chromosome 2;
(18) QTL region 18, extending from nucleotide 50449700 to 50857310 of chromosome 2;
(19) QTL region 19, extending from nucleotide 50879601 to 51539414 of chromosome 2;
(20) QTL region 20, extending from nucleotide 52821582 to 52960520 of chromosome 2;
(21) QTL region 21, extending from nucleotide 42585292 to 42728875 of chromosome 3;
(22) QTL region 22, extending from nucleotide 9561644 to 9701199 of chromosome 4;
(23) QTL region 23, extending from nucleotide 12469969 to 13409114 of chromosome 4;
(24) QTL region 24, extending from nucleotide 14672228 to 14789226 of chromosome 4;
(25) QTL region 25, extending from nucleotide 395189 to 842107 of chromosome 5;
(26) QTL region 26, extending from nucleotide 47205529 to 47293291 of chromosome 5;
(27) QTL region 27, extending from nucleotide 48857594 to 48932286 of chromosome 5;
(28) QTL region 28, extending from nucleotide 5943980 to 6002717 of chromosome 6;
(29) QTL region 29, extending from nucleotide 6337822 to 6563232 of chromosome 6;
(30) QTL region 30, extending from nucleotide 6818733 to 7281658 of chromosome 6;
(31) QTL region 31, extending from nucleotide 17578027 to 18209857 of chromosome 6;
(32) QTL region 32, extending from nucleotide 26204516 to 26755007 of chromosome 6;
(33) QTL region 33, extending from nucleotide 36492757 to 36494757 of chromosome 6;
(34) QTL region 34, extending from nucleotide 219790 to 1533149 of chromosome 7;
(35) QTL region 35, extending from nucleotide 8700733 to 9242332 of chromosome 8;
(36) QTL region 36, extending from nucleotide 23767318 to 23957652 of chromosome 8;
(37) QTL region 37, extending from nucleotide 26648547 to 26848102 of chromosome 8;
(38) QTL region 38, extending from nucleotide 606020 to 1309231 of chromosome 9;
(39) QTL region 39, extending from nucleotide 3499347 to 3638435 of chromosome 9;
(40) QTL region 40, extending from nucleotide 28437588 to 28513671 of chromosome 9;
(41) QTL region 41, extending from nucleotide 28581068 to 28912034 of chromosome 9;
(42) QTL region 42, extending from nucleotide 32327318 to 32434321 of chromosome 9;
(43) QTL region 43, extending from nucleotide 32538074 to 32540074 of chromosome 9;
(44) QTL region 44, extending from nucleotide 32775289 to 33054696 of chromosome 9;
(45) QTL region 45, extending from nucleotide 33133902 to 33254107 of chromosome 9;
(46) QTL region 46, extending from nucleotide 15342814 to 15405953 of chromosome 10;
(47) QTL region 47, extending from nucleotide 15933273 to 15943963 of chromosome 11;
(48) QTL region 48, extending from nucleotide 12178551 to 12249693 of chromosome 12;
(49) QTL region 49, extending from nucleotide 2052746 to 2447722 of chromosome 13;
(50) QTL region 50, extending from nucleotide 14345084 to 14709650 of chromosome 13;
(51) QTL region 51, extending from nucleotide 22031000 to 22147560 of chromosome 13;
(52) QTL region 52, extending from nucleotide 23588504 to 24307350 of chromosome 15;
(53) QTL region 53, extending from nucleotide 1511530 to 1596020 of chromosome 16;
(54) QTL region 54, extending from nucleotide 2684531 to 2803682 of chromosome 16;
(55) QTL region 55, extending from nucleotide 5535711 to 5995857 of chromosome 16;
(56) QTL region 56, extending from nucleotide 8379248 to 8554851 of chromosome 16; or
(57) QTL region 57, extending from nucleotide 8883687 to 9269845 of chromosome 16.

2. The method of claim 1, wherein the high-oil-production trait comprises increased oil per palm plant.

3. The method of claim 1 or 2, wherein the population of oil palm plants comprises an Ulu Remis dura×AVROS pisifera population, a Banting dura×AVROS pisifera population, or a combination thereof.

4. The method of claim 1, 2, or 3, wherein:
the population of oil palm plants comprises an Ulu Remis dura×AVROS pisifera population;
the first QTL corresponds to one of QTL regions 7, 8, 13, 14, 16, 18, 19, 25, 33, 52, or 54;
step (iii) further comprises applying a genotype model, thereby predicting the palm oil yield of the test oil palm plant; and
the first SNP marker is associated, after stratification and kinship correction, with the high-oil-production trait with a genome-wide −log₁₀(p-value) of at least 4.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 4.0 in the population.

5. The method of claim 1, 2, or 3, wherein:
the population of oil palm plants comprises an Ulu Remis dura×AVROS pisifera population;
the first QTL corresponds to QTL region 8;
step (iii) further comprises applying a dominant model, thereby predicting the palm oil yield of the test oil palm plant; and
the first SNP marker is associated, after stratification and kinship correction, with the high-oil-production trait with a genome-wide −log₁₀(p-value) of at least 4.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 4.0 in the population.

6. The method of claim 1, 2, or 3, wherein:
the population of oil palm plants comprises an Ulu Remis dura×AVROS pisifera population;
the first QTL corresponds to one of QTL regions 8, 13, 18, 22, 23, or 45;
step (iii) further comprises applying a recessive model, thereby predicting the palm oil yield of the test oil palm plant; and
the first SNP marker is associated, after stratification and kinship correction, with the high-oil-production trait with a genome-wide −log₁₀(p-value) of at least 4.0 in the population or has a linkage disequilibrium r² value of at least 0.2 with respect to a first other SNP marker that is linked thereto and associated, after stratification and kinship correction, with the high-oil-production trait with a genome-wide −log₁₀(p-value) of at least 4.0 in the population.

7. The method of claim 1, 2, or 3, wherein:
the population of oil palm plants comprises a Banting dura×AVROS pisifera population;
the first QTL corresponds to one of QTL regions 1, 3, 4, 5, 6, 9, 10, 11, 12, 21, 26, 27, 28, 29, 30, 31, 32, 34, 35, 36, 37, 38, 40, 41, 42, 43, 44, 47, 49, 50, 51, 53, 55, or 56; and
step (iii) further comprises applying a genotype model, thereby predicting the palm oil yield of the test oil palm plant.

8. The method of claim 1, 2, or 3, wherein:
the population of oil palm plants comprises a Banting dura×AVROS pisifera population;
the first QTL corresponds to one of QTL regions 17, 20, 49, or 55; and
step (iii) further comprises applying a dominant model, thereby predicting the palm oil yield of the test oil palm plant.

9. The method of claim 1, 2, or 3, wherein:
the population of oil palm plants comprises a Banting dura×AVROS pisifera population;
the first QTL corresponds to one of QTL regions 2, 5, 9, 10, 15, 17, 24, 26, 27, 28, 29, 31, 32, 34, 35, 36, 39, 41, 44, 46, 47, 48, 50, 51, 56, or 57; and
step (iii) further comprises applying a recessive model, thereby predicting the palm oil yield of the test oil palm plant.

10. The method of any one of claims 1-9, wherein the test oil palm plant is a tenera candidate agricultural production plant.

11. The method of claim 1 or 2, wherein the population of oil palm plants comprises an Ulu Remis dura×Ulu Remis dura population, an Ulu Remis dura×Banting dura population, a Banting dura×Banting dura population, an AVROS pisifera×AVROS tenera population, an AVROS tenera×AVROS tenera population, or a combination thereof.

12. The method of claim 1, 2, or 11, wherein the test oil palm plant is a plant for mother palm selection and propagation, a plant for introgressed mother palm selection and propagation, or a plant for pollen donor selection and propagation.

13. The method of any one of claims 1-12, wherein the test oil palm plant is a seed, a seedling, a nursery phase plant, an immature phase plant, a cell culture plant, a zygotic embryo culture plant, or a somatic tissue culture plant.

14. The method of any one of claims 1-12, wherein the test oil palm plant is a production phase plant, a mature palm, a mature mother palm, or a mature pollen donor.

15. The method of any one of claims 1-14, wherein:
step (i) further comprises determining, from the sample of the test oil palm plant, at least a second SNP genotype of the test oil palm plant, the second SNP genotype corresponding to a second SNP marker, the second SNP marker (a) being located in a second QTL for the high-oil-production trait and (b) being associated, after stratification and kinship correction, with the high-oil-production trait with a genome-wide −log₁₀(p-value) of at least 3.0 in the population or having a linkage disequilibrium r² value of at least 0.2 with respect to a second other SNP marker that is linked thereto and associated, after stratification and kinship correction, with the high-oil-production trait with a genome-wide −log₁₀(p-value) of at least 3.0 in the population; and
step (ii) further comprises comparing the second SNP genotype of the test oil palm plant to a corresponding second reference SNP genotype indicative of the high-oil-production trait in the same genetic background as the population,
wherein the second QTL corresponds to one of QTL regions 1 to 57, with the proviso that the first QTL and the second QTL correspond to different QTL regions.

16. The method of claim 15, wherein step (iii) further comprises predicting palm oil yield of the test oil palm plant based on the extent to which the second SNP genotype of the test oil palm plant matches the corresponding second reference SNP genotype.

17. The method of claim 15 or 16, wherein:
step (i) further comprises determining, from the sample of the test oil palm plant, at least a third SNP genotype to a fifty-seventh SNP genotype of the test oil palm plant, the third SNP genotype to the fifty-seventh SNP genotype corresponding to a third SNP marker to a fifty-seventh SNP marker, respectively, the third SNP marker to the fifty-seventh SNP marker (a) being located in a third QTL to a fifty-seventh QTL, respectively, for the high-oil-production trait and (b) being associated, after stratification and kinship correction, with the high-oil-production trait with a genome-wide −log₁₀(p-value) of at least 3.0 in the population or having linkage disequilibrium r² values of at least 0.2 with respect to a third other SNP marker to a fifty-seventh other SNP marker, respectively, that are linked thereto and associated, after stratification and kinship correction, with the high-oil-production trait with a genome-wide −log₁₀(p-value) of at least 3.0 in the population; and
step (ii) further comprises comparing the third SNP genotype to the fifty-seventh SNP genotype of the test oil palm plant to a corresponding third reference SNP genotype to a corresponding fifty-seventh reference SNP genotype, respectively, indicative of the high-oil-production trait in the same genetic background as the population,
wherein the third QTL to the fifty-seventh QTL each correspond to one of QTL regions 1 to 57, with the proviso that the first QTL to the fifty-seventh QTL each correspond to different QTL regions.

18. The method of claim 17, wherein step (iii) further comprises predicting palm oil yield of the test oil palm plant based on the extent to which the third SNP genotype to the fifty-seventh SNP genotype of the test oil palm plant match the corresponding third reference SNP genotype to the corresponding fifty-seventh reference SNP genotype, respectively.

19. A method of selecting a high-palm-oil-yielding oil palm plant for agricultural production of palm oil, the method comprising the steps of:
(a) predicting palm oil yield of a test oil palm plant according to the method of any one of claims 1-18; and
(b) field planting the test oil palm plant for agricultural production of palm oil if the palm oil yield of the test oil palm plant is predicted to be higher than average for the population based on step (a).

20. A method of selecting a high-palm-oil-yielding oil palm plant for cultivation in cell culture, the method comprising the steps of:
(a) predicting palm oil yield of a test oil palm plant according to the method of any one of claims 1-18; and
(b) subjecting at least one cell of the test oil palm plant to cultivation in cell culture if the palm oil yield of the test oil palm plant is predicted to be higher than average for the population based on step (a).

21. A method of selecting a parental oil palm plant for use in breeding to obtain agricultural production plants or improved parental oil palm plants, the method comprising the steps of:
(a) predicting palm oil yield of a test oil palm plant according to the method of any one of claims 1-18; and
(b) selecting the test oil palm plant for use in breeding if the palm oil yield of tenera progeny of the test oil palm plant is predicted to be higher than average for the population based on step (a).


SEQUENCE LISTING (114 sequences)

SEQ ID NO: 1 - DNA, 240 bp, Elaeis guineensis
  misc_feature (1)..(240): Elaeis guineensis linkage group LG1 18204491 to 18204730
tgatgtgcctttttcgtagctatattcatccggagacaaagcatcttcaaggaatccgttgtggctatatttctctagggacgaagcatccttctcgtggatatattcatccggagacgaagcatcttcgaggaactcgttgacgtcaaaggattagatgtgaggcaacataaaacaaatctaattcaatttttttatgacatcatgttcagccagcagtatataaccacatgtgcctat

SEQ ID NO: 2 - DNA, 240 bp, Elaeis guineensis
  misc_feature (1)..(240): Elaeis guineensis linkage group LG1 18358162 to 18358401
ctcctcgtccttagataggttgagtgagagttggtaaaagttaatgtcggccaacggacatcccctatccttgaacaaatcgaagaagagaccttaaaacgccgattggctgcagtgctcttcctcttattgcaaaatcttgaatatcgacatcggaggtcggcaaaagccaatgcctatcgcaaaatacttcattttcaaaccaagacaaaagggatgacgatgcaccaaactccaaac

SEQ ID NO: 3 - DNA, 240 bp, Elaeis guineensis
  misc_feature (1)..(240): Elaeis guineensis linkage group LG1 18922390 to 18922629
tgatagtttcttccaatcatttcctaaaggtgttagaatttttctttctttgcatgaaaaaatgggataaagttgtttagattagataataaccaacacattagcatgagtgcaagcaaatatcccaagtccaattaagatgttgcttgtttgttacactaaacctgctttcgacatgaagcaatcctagatgcttcaaactaaaaatatttcatttttatagttatgaagtatttcttc

SEQ ID NO: 4 - DNA, 240 bp, Elaeis guineensis
  misc_feature (1)..(240): Elaeis guineensis linkage group LG1 19167684 to 19167923
acaatagaggcctcacgatgattcaaatttttggatgtgatatattatttttaaactatttatatacaaaatgctatataatctaaaaaattctaatctattttcataaatttccaacctaaaagtttaggctattaagaggaggactgactcatgtgaataaacctatgtgacattcttttattagtcaatataagtttatgataattatctccctcacccattctcgtgacaccctcg

SEQ ID NO: 5 - DNA, 240 bp, Elaeis guineensis
  misc_feature (1)..(240): Elaeis guineensis linkage group LG1 19188077 to 19188316
cctttctcactctattgaataataccatgccaaatcaagaagatcatcagatcgactgttttatatcttttgatggtattggtgaagcaggcaagtagctgataatgaactacatagctggctgaaaaccactgcatattgttaactaagcttgcctcagaataaggattccaattttgttgtgtttatgatatatttttgtaatcgacattggaattgtttagagttaattttttagtg

SEQ ID NO: 6 - DNA, 240 bp, Elaeis guineensis
  misc_feature (1)..(240): Elaeis guineensis linkage group LG1 19684841 to 19685080
ataatcataattttatatgaaaaataaattgttagaagatgcattactggcaaaagaagacgtaggactcatccgcggatggaaataaggtattcaagagactatcacactcgtgcacgaaaaaggtaacactgctcgatcgaaatggagagtgagatggaggtcgtcgtcagaatttcgtcagaaaataataatcgaaagtaagagatgccaaacaacagatattgaaatagagagaat

SEQ ID NO: 7 - DNA, 240 bp, Elaeis guineensis
  misc_feature (1)..(240): Elaeis guineensis linkage group LG1 23276098 to 23276337
gtacttcaattcatatgtcgagtctaaggcactggagagattccatcttccagcagataaggaagccctcaccaattatgtcttcatgatacatacgattctcatgcggttgcttttgatgatgtcgcaaccttcctgggatcattctcaacacgcagactgttttctctccgataaagagatttcgcaccctaattaaatggcaacaaattagacaatttactcacattgatgattgca

SEQ ID NO: 8 - DNA, 240 bp, Elaeis guineensis
  misc_feature (1)..(240): Elaeis guineensis linkage group LG1 23456531 to 23456770
ccacttttctactttcatccaaaaagaactcccgttaataagatgaatacaatgtaaagatcttacatgaactgaatctgatctctttataccctaaaggcctactctgccaatatcaacggtaataatggaaaaatatcgtgatgagaccttaccagagtagtttgttgtggaaccgtaaatactcaaaggaaaggggaaaggaaaggttggagatctctggaatgcaaggaggaagca

SEQ ID NO: 9 - DNA, 240 bp, Elaeis guineensis
  misc_feature (1)..(240): Elaeis guineensis linkage group LG1 26021716 to 26021955
cctacattgtgaaccgatgatcatggatatcttctgaaggtttcttaaaatcacacaaacgtctgacctctatgggtatctacacgaggtctcgatctgatcagaagcttcttgatctcaccaggatgctagcttctttgcagagatcatatctcgatggttgaaaaaattttttttttctcaagaccctcttagagaagaagaaaaaaataggaggacgaagatctctatgctagagat

SEQ ID NO: 10 - DNA, 240 bp, Elaeis guineensis
  misc_feature (1)..(240): Elaeis guineensis linkage group LG1 26066295 to 26066534
gtgtaacaattatatgggtggtgattaattattatttgtaactactgcaaaacttccttaggtgccattaccttcgctagtcaccgcaacaacgaatacaaggaaggcaaggactgggagcttcctttatttgatttggggacaatcattgtagcgaccaacaacttctcaatcgaaaacaagcttggaaagggtggcttcggccccgtgtacaaggtattttcttaatttaccaaccga

SEQ ID NO: 11 - DNA, 240 bp, Elaeis guineensis
  misc_feature (1)..(240): Elaeis guineensis linkage group LG1 28110016 to 28110255
gcgcagactggaagattcttcttgttgtccaatcttctatcatagatctcatgcttagttgtttaaaatcctgcgcaacatgggctgtcttctccaagaaggggttgtgtgcgaatttttttttctttaagaggtcaacgagagatccttcagtgtccacctgtttataaacattgttccccaacttatattcctgcactccatggagagaccatcatatataacatagttgcaccttaa

SEQ ID NO: 12 - DNA, 240 bp, Elaeis guineensis
  misc_feature (1)..(240): Elaeis guineensis linkage group LG1 28234560 to 28234799
cagacccagctgactctctcttcatccataaatggtgttcttttttcccactcttcttcaactttctgcaatccacatgaaggtaaacgaatttattatcaaaatggaaacgaaatatttactaatggaaaagaagtggcagactccaaactatcacatttttattgaaatatgggaaaatcatccaaaaacttcataggtgtggttagaggtccaatccttcatctagatttaaatgaa

SEQ ID NO: 13 - DNA, 240 bp, Elaeis guineensis
  misc_feature (1)..(240): Elaeis guineensis linkage group LG1 29798161 to 29798400
tcatgctatcattagacttattttcggccacattacttcctaaggttacaaaaaattcttgcaggaaggtactcctaggttaatgcggagactaaatttgatataacattgcaacgaaggacaagcctgattaaaggacaatggtggccaaggctaagaaacctagtgcgtgactggcttaaagcaccaggccagggccagcatggggaaaaccccgcccgggtatgtgaggtttgaaag

SEQ ID NO: 14 - DNA, 240 bp, Elaeis guineensis
  misc_feature (1)..(240): Elaeis guineensis linkage group LG1 30164090 to 30164329
gtccaattgataggaggttccagtcagtatttaattatcaaaaagcacaatattaaattgcaatgttgagagtttatatgtaacagtattcagagaaatggaagcgttaagaagtgaaaaatgtttccatagatcagctaattgatttattcatgaatttcctagtattcacaaatcgatacactatattgatcatattattatcttataagacaaagtaagaagagaagaaaagcaccc

SEQ ID NO: 15 - DNA, 240 bp, Elaeis guineensis
  misc_feature (1)..(240): Elaeis guineensis linkage group LG1 30684639 to 30684878
ggcttcatgaatccaccataactacaataaccattttgataagccaaataaaatacatcactcatatttgtgtagcctaaacatcagattataaacaaagtatactggctgacaaacatgttagtaacaagatatctcatatatgagacttaattccatgtgaaaaacatagaaaaccttccttagaaggactcagacttagtgctaatattttaagccagcatttgaaaatcataatga

SEQ ID NO: 16 - DNA, 240 bp, Elaeis guineensis
  misc_feature (1)..(240): Elaeis guineensis linkage group LG1 31159890 to 31160129
tttatatgtgacattttctcgggtgagggaatctgtattcagtttaaaattaatcattcatgcctgtgaattttacaagccagtttccacgttgaaacatcacagcatcaaaagtgcagcatgatgttaggttgactgtcttgcttaccacgtataataacttgtatccatatgctcaccttacccctcaaactatccaatagttatactcagtcatcttggaattctaaaattattgtt

SEQ ID NO: 17 - DNA, 240 bp, Elaeis guineensis
  misc_feature (1)..(240): Elaeis guineensis linkage group LG1 37811723 to 37811962
gacactctagaaatgcatgagttcacaaatttgttttcgtaccattttccaggaagaactgcttcacatgtcgatttgtattatcttgatattgtatcgaagaatttgaggatacacagggtttatatccttactatattctatgatggacttaccttatacctatcatttaggcatataggaaacaatgaaagattgcttccattaaaaaggggaagtgcattcaacaatataaaagca

SEQ ID NO: 18 - DNA, 240 bp, Elaeis guineensis
  misc_feature (1)..(240): Elaeis guineensis linkage group LG1 38636990 to 38637229
ataaataataatggaatcttaggtcaatctagtagcattaagaaagatagaagtccatgattgatgtaatgtgttcaaaggaatgattgaatcttaacaaaatctgtttgaaaacttgtgagcaaattatttttgtaaatccttgccaattgttttttatttgacatggaatttttgaaataagcactaatggaggattaggctatcaagagaacccctgctccacaaaatgcagtccta

SEQ ID NO: 19 - DNA, 240 bp, Elaeis guineensis
  misc_feature (1)..(240): Elaeis guineensis linkage group LG1 38659012 to 38659251
attttcgattcttctcttccatgaaaaaatagctttatgaacgttagcaagtaatagagattacttcaccttaggtgagtgatgaccttgatattgctaagcttgttgaacaacttccaagactccaggattttcaaatcaaaatggaatagagcctctactaatcatattcattatggagataagtaagggataatgatcagaggcaaatcgagtaaggtgtgatacagtggattcagg

SEQ ID NO: 20 - DNA, 240 bp, Elaeis guineensis
  misc_feature (1)..(240): Elaeis guineensis linkage group LG1 39206413 to 39206652
agatgcttcaacatgattactactggttgatattgcgatgaggcagtcaatctgatacagaagtgtgatagatgccagagaaatgctcacatccaaagccgacctaccactcccttgacatctattggtgcaccctggcccttcattcaatggtgaatggatattcgtaggtgctgtttggtatatcaccaatgagatgatcactaagtgatgtgatcactagggtgatatgaatactaa

SEQ ID NO: 21 - DNA, 240 bp, Elaeis guineensis
  misc_feature (1)..(240): Elaeis guineensis linkage group LG1 39243858 to 39244097
tgtcaggtgagccaacgggactgttgtgccatgtcatatcgctggttccccatttcctggtacgtccttctttattgcccaaacatatgagatatggaggatagctgtttgtataaaggaaagaaagggtggaggaaaccaaatatgataatgggtagggtgaggaagagtatacttcaaagtggtgaagggagaaaaacaaaatcagaaaagaagaaaggaaaaaataagaatgatatg

SEQ ID NO: 22 - DNA, 240 bp, Elaeis guineensis
  misc_feature (1)..(240): Elaeis guineensis linkage group LG1 39841918 to 39842157
ccatgcctcctccttcaatacggagagctacgtcgacaacaagcaacacgacgatgtgcggtgggccaacatcatcgtcgctcgtgtggtggcgaacgttgtgcgcaccagcctcggccccaagggcatggacaagatgatcgcctccgacgaggttatcattaccaacaacggagccaccatccttaacaagatggaggtcctccagcccaccgccaagatgctcatcgacctctttgg

SEQ ID NO: 23 - DNA, 240 bp, Elaeis guineensis
  misc_feature (1)..(240): Elaeis guineensis linkage group LG1 61305818 to 61306057
ggacacagatttttgtgggttcaataattttttttagacatcaaccattggatagcatcttgcacagattttagagcacttcaactccttcctttcatctcgaagccttcattatgctatctgatggttggtgttcaaaaagaaggtgaatgcttattttgggcgaaagaaacaacctgtacccatatttttttcactcggatctttcctttccaatttggaattgatcacaagcttctt

SEQ ID NO: 24 - DNA, 240 bp, Elaeis guineensis
  misc_feature (1)..(240): Elaeis guineensis linkage group LG1 61571867 to 61572106
gaaatcaatttgttttgtgggactagaattattatgcagcaaagtcatatcagtatcaatgttactttgtcccacagtcaatttggtacacactcatatctttgtttttggctataatattaaagatcatagtagaggtttactaaacaaaatataaatgttgctatatctttgaagcaagcaaaaggctggcattcccctggagtgaatgctccatttctgtttttatgccggacctgt

SEQ ID NO: 25 - DNA, 240 bp, Elaeis guineensis
  misc_feature (1)..(240): Elaeis guineensis linkage group LG2 1068379 to 1068618
gtatgtatgtgttctctttttattttaattcatatatgtcaatattctcttcttacagctccggttctgtttagtgcctactagattttgttcaattctacttacaagaggacgaactggaagacttcactatcaaactacattcctgtcatgtatcctttatttttgttacagttaaaaaccagccattggtgtaatcccttgaggaaagatcttctgcttgttgtcatggacgtactc

SEQ ID NO: 26 - DNA, 240 bp, Elaeis guineensis
  misc_feature (1)..(240): Elaeis guineensis linkage group LG2 1516332 to 1516571
tttttccctttttttcttttttttttttgcgataacaagagcgcaattgaaggcagtaaggagggaagaagagatagggagaccttgagggtgccattacaagcgcaaggggcttccatctcgctctcctccccttcttcttggcatatcctgcactctataacatctccacccttcttcttcttcttagactgagccttcttctcggaagacccctcctcgccccgaccaacatcgacc

SEQ ID NO: 27 - DNA, 240 bp, Elaeis guineensis
  misc_feature (1)..(240): Elaeis guineensis linkage group LG2 1616491 to 1616730
ggtaccacaactgcataaaaggactaaatcagaaattgctacatataagataatatcaggattgtagtcaagaagcaaaatgaaaagaaggttttatgccaagcatgtggagagataaagagagaaaccatttgctttcaaactattgatctagtagcgctaaaagtagaatttattaagttttgacttcaaatgctgtaaaataatgttaattaaatagattcgtagaaatgcttgtag

SEQ ID NO: 28 - DNA, 240 bp, Elaeis guineensis
  misc_feature (1)..(240): Elaeis guineensis linkage group LG2 2015930 to 2016169
agataatgtgcccatttgagaatctgaatatgcatgtaaatggtaagaagactgatcggaacactgcaataaacatttagtggctatttaagcagctttttatgaattaaatattaggaagatcttgtattcacatgaaatggtgttgagtgttgcttatgatttgatgtaaataggagggatgattcatggtttgcagcactaggaagatcttgcattcacatgacttggagtgatcgc

SEQ ID NO: 29 - DNA, 240 bp, Elaeis guineensis
  misc_feature (1)..(240): Elaeis guineensis linkage group LG2 17637996 to 17638235
tgccaacaacattaattttctatggagcaagataccagaacttttaaaaaatatcaaataatcagctatatttcctcaaatcacttaaattatgttaagaaagaggaacatgacaaagatagagatttgcttgtcatcagttggattttgataggttcagtaaaattctcaatttatgcagtgcccatttcaccacatataccatttggttcgtaaaatatattagtgacaatttcccaa

SEQ ID NO: 30 - DNA, 240 bp, Elaeis guineensis
  misc_feature (1)..(240): Elaeis guineensis linkage group LG2 17959672 to 17959911
attcaatgatgaaaacattcccacactttccactcattagcttggggaatcgacaaacatatagaattatagattgtctcgcatggcacattgttgaaaataattaactagcttcaacaagaacagattgaacaataaacaatatcttacctattgctttgacgaaagtcgtaaccaaagccaaacaatgtggttcctttctcatatccacattcaacaagcatatcaatcatatcatga

SEQ ID NO: 31 - DNA, 240 bp, Elaeis guineensis
  misc_feature (1)..(240): Elaeis guineensis linkage group LG2 20732085 to 20732324
gaaacaagaaaaaacccaaaccatcagactgaaccagtttcatctacatggctcacagaagagtaggctgcatatgtggctcatagtgctagaagagctgcataaggagctgcagaacgttaaaagtgctgtatatgcatttagtattaggtggaccaccttcgggcatatatgtagtatcatctcatataataaccttatatcagcaatgtggtgaagtcaaggggctgaaactctgag

SEQ ID NO: 32 - DNA, 240 bp, Elaeis guineensis
  misc_feature (1)..(240): Elaeis guineensis linkage group LG2 20977251 to 20977490
ggtgagctgagcaccagcactggccagcaagatggattccccattttcttctccacacggcatcagaagggcagctactggaatatatctgttccatggcccacacctcatcaaatatctggtataatatggtacgagagcaacaggtggtggcgagaaacatacttcctagctccccaccgagataccgttccttttctatccatcccaagtctcgttacaaattccacgaaaaccaat

SEQ ID NO: 33 - DNA, 240 bp, Elaeis guineensis
  misc_feature (1)..(240): Elaeis guineensis linkage group LG2 31844836 to 31845075
ataccacctagctctaataccacttgttgtgcaggatctggtatcatacattgcaacaggaagaaagaagaaataaaacaagaaacacaatcaaatacgtggatcagccaacaggctcacttctacggggcatgcaagcttcactatgaaaaaaaaatttacaaaagaagatcacaccctcaatcctcatataccaatccctctatctcaacaagaagctctcctttacaaaagctccct

SEQ ID NO: 34 - DNA, 240 bp, Elaeis guineensis
  misc_feature (1)..(240): Elaeis guineensis linkage group LG2 31979832 to 31980071
ctttcttcgagataaagatccccgaactatttgcctagacaccgatcgatcagtcagttagaacccaaaccacccttgggcttccttgtcctagtaagctcgaatgttcctagggtcagcatctaattgatttcactttaggcttgggttatgcaaatgcagggggatagtttatgggctaagaaagggtgatcaaatcctcaccttatcttgtattaggtttaatactcttaaaatcaa

SEQ ID NO: 35 - DNA, 240 bp, Elaeis guineensis
  misc_feature (1)..(240): Elaeis guineensis linkage group LG2 50449700 to 50449939
gtttggtacacaattcagcttcccaatatagcggattttgctcctgaattatgtccactcctacttgtagtgaccaaagtcgttcacatcaaatctaataatccttctggatcaacttttttctagattctattcttgaaaacatcttagtaatagccctagaacatctcttacctatcatcatatcatgtgctatcttaagtttttaacttaatatgacggttactatcatcctaaata

SEQ ID NO: 36 - DNA, 240 bp, Elaeis guineensis
  misc_feature (1)..(240): Elaeis guineensis linkage group LG2 50857071 to 50857310
tctttgtgtttattctccaatattattgtttattatgaatgagccccagtcgaattcctgtttatttctatggtatttatatttctcaccactgactaggtatttatcctaaaaggtttgtattttttttagagtggacgtccaatgagggagaggaccctagacgggcataggtccaacaattaatgactaacctttttttaaatgatgtggtagtttttacaatgaagagagagtgaa

SEQ ID NO: 37 - DNA, 240 bp, Elaeis guineensis
  misc_feature (1)..(240): Elaeis guineensis linkage group LG2 50879601 to 50879840
gcaggtcaaatccaaacaactttcagaaaatcggaaaaattacaataagatgattaatgtgcaaaaaaagagaaagatcaaactgacagtcaatttagcagttgtggacaatctggcaaagatcctgtacccgcgctcaatccgaacgtgtgttaaattcaaattgggcttgaccacaactcaagtaatttcaaatccacaagctgttaatgttttaaaaaacaattacaaatttcgata

SEQ ID NO: 38 - DNA, 240 bp, Elaeis guineensis
  misc_feature (1)..(240): Elaeis guineensis linkage group LG2 51539175 to 51539414
acatgtggatgcaagtcaaaagatgtcttgattccagtagcttataatccaagacctacagtatttacaaaaactatggtaagttgatcaatacaggaaataaattctgaaataatccaaggttccaaagaagttgttcaaatgctatgcagccaatatatgacatcaagaagggaaaataattgttatcacagccaactaaaacatcctaacattacaatggaaaataatttcaatgcg

SEQ ID NO: 39 - DNA, 240 bp, Elaeis guineensis
  misc_feature (1)..(240): Elaeis guineensis linkage group LG2 52821582 to 52821821
gaaattctggacgatgtggggaagatcggagggatcgattggagagaattcagattttgtgaggtgggggatcgatgggacaggcgatgtcagtctctctgtatctctccttgtctccctttcgatgagagatcgacggaaggagcaacatcgatctctctatctctccttgtctcactttcgacaggagatcgatgggaggagcgatgccgatctctccctatctcttctgattctact

SEQ ID NO: 40 - DNA, 240 bp, Elaeis guineensis
  misc_feature (1)..(240): Elaeis guineensis linkage group LG2 52960281 to 52960520
catgactcaattatgttttagtgtatccgaaatggtgaggatgtatggtccatgaatatttcccctccttgaatatttctcaccagatggaaaacagtttaatccagtgtctcttgtcaacaaacaacttccaaacttcaacttgagttctgatgctgcaatctgatcttcaaccaagggctctgtgtccccagctgtttctgacaatttggtgtactcttcaaccaattgatctctgta

SEQ ID NO: 41 - DNA, 240 bp, Elaeis guineensis
  misc_feature (1)..(240): Elaeis guineensis linkage group LG3 42585292 to 42585531
tttcttggaaagcagtgagggtactatttacttatcgaatggatcgagttatatgatcagaggcatcgaaacggtcagctggaggatatatgatggtatagtgaggaggttggaggaggtccgatacatatccaatttcagatgaaattttatctcactaagcagactagattcgagaggctagatgatgatagttggtggagaaattttgaaggtgttgcacggcaatagggttattct

SEQ ID NO: 42 - DNA, 240 bp, Elaeis guineensis
  misc_feature (1)..(240): Elaeis guineensis linkage group LG3 42728636 to 42728875
tacctccaattattatatctagaataattcaaatgtactgatttttcgcctctcattttaaacggataaactccaaactatttttcttaatatataaccaagctcaaattcatcgaggaagggaacatatataaagagaaggcatatgtagtgtacagtgacaatatagtcctctcgaagtaataatgtatcttccattcaatgtctcctagctttaatgctgtagtgaccgccttgcac

SEQ ID NO: 43 - DNA, 240 bp, Elaeis guineensis
  misc_feature (1)..(240): Elaeis guineensis linkage group LG4 9561644 to 9561883
ctgataggatcttttatattctgattttctggactgctttaacactagagtggtgtggtcgtccgcatcaatcaaaaaaaataaatctaaatattttttcttaagataaaaaaagaaaaaatataacccggtacttgttgaatcgtttcttactttctcttaactatggtaaaaagaaaaaaaatattttctctttatctctatattatatctcaattttagattcttcatttctatttt

SEQ ID NO: 44 - DNA, 240 bp, Elaeis guineensis
  misc_feature (1)..(240): Elaeis guineensis linkage group LG4 9700960 to 9701199
cagcatgcctcaacctcagaccaccaatttcttaatgcagaatccctgatcattctcagaatcaataatatcagcacaaacattaagtgtccgggagagagttgttccctcaatcctatctatggattctattgcaaagtatgatatttttttcaatcataattaacaagcagtaaatttatgtaacttaaacttttaagacttgattaaaattttggctgatacgattttgtgccacat

SEQ ID NO: 45 - DNA, 240 bp, Elaeis guineensis
  misc_feature (1)..(240): Elaeis guineensis linkage group LG4 12469969 to 12470208
aagggtttctactcctcagtgagatgtctatctcccataattaattagttggttctttcctttgctggacgcaatggttgaaaaggtcaatggttacacatcacaatgtgtggcaaataactactgaattgcaaaaaaaaggtcctccattcttcgccaaatggtgctaaaactatccgtaaatttggtgttatgtctgcttaccagaagtaaaacaaccaaatatgatgatttcacata

SEQ ID NO: 46 - DNA, 240 bp, Elaeis guineensis
  misc_feature (1)..(240): Elaeis guineensis linkage group LG4 13408875 to 13409114
aggttcagattcactgtttggagtaggatgcacctagatgggatctatcgaccttaatagaaaaagaagtcctatgcgatttgtgagactgagttcagaaagtctttggtcaaagcaaatatgaatactgaaaaagagttttcacagtattcacaaataaactcgagtcgagccaatctagcatataactgacaataaggtctgacaagctctccatgatcttcatcaagtcagaactcg

SEQ ID NO: 47 - DNA, 240 bp, Elaeis guineensis
  misc_feature (1)..(240): Elaeis guineensis linkage group LG4 14672228 to 14672467
aatgaagaacttccagatttgaattgcatgaaagcatgcaacttggagttggctcattgtttattaattttggtagcagagaaggttttcttcttcttcttcttcttcttcctttctttttttttttttttttctctccatatatacaattttgtatgattttgaagctctgttctttcccaaatcacaaaaattaaaggcatatttctctaagtcgaagacttttccatcaaattgaag

SEQ ID NO: 48 - DNA, 240 bp, Elaeis guineensis
  misc_feature (1)..(240): Elaeis guineensis linkage group LG4 14788987 to 14789226
ggttcaggcatgggttcttgggggtgttttaacatagaaaaatcagatttaagatgtgggtttgaggagccctttcaatgtgagaaagcagctggttttacgagaaaaatcagagaaaggctccttccttggcatgagaaaaagggagagtaagaaagtcttcctcttgggctgaagaaaaggaaaaggcatctttcttcttgtgcataattttgagagagaaaaggttcctcttaaaga

SEQ ID NO: 49 - DNA, 240 bp, Elaeis guineensis
  misc_feature (1)..(240): Elaeis guineensis linkage group LG5 395189 to 395428
taaatttattcctatatccccagccttagctctaggattcgattaaagtgtgattggtttgaagggagttggcctctagaatgaaaactgaaataaataacttcaatttcagctatttggccagagagagtcttatttctattttgattcaagagaaaaataggaaccaaatgcattgacgatataaagggtccaattcaatccaattctgatttcatttgtgatttcgactgcgaacca

SEQ ID NO: 50 - DNA, 240 bp, Elaeis guineensis
  misc_feature (1)..(240): Elaeis guineensis linkage group LG5 841868 to 842107
gacaaggtggggtattgaagctaattttgtgaaaattaagataattttggatatgaagcccccaacttctaggtgagacatccaaaaattgatcgggtgcattgcatctctcagccaattcatttctaagtcggcagagcaatgccttccattcttcaaaactctcaggcaaatgaaagactttacttgtatagaagaatgtcaaaaagctttcaaagacttgaagctatatttaagctt

SEQ ID NO: 51 - DNA, 240 bp, Elaeis guineensis
  misc_feature (1)..(240): Elaeis guineensis linkage group LG5 47205529 to 47205768
gatcaactgcacagtcacatctgtctggctatctatagcttctcttatttttctaacagtcgctggatctgtttcagggccctggagaaatctgaaagttgcccaggaaaaaacaacagtttaaaagcaatcaggtgaagaaaattttaaaaactttatcctggatcagaaaaagtagcttgtgatattaaataggctgataataaaaaaatattaaagtatctaccggtccaaatgtgc

SEQ ID NO: 52 - DNA, 240 bp, Elaeis guineensis
  misc_feature (1)..(240): Elaeis guineensis linkage group LG5 47293052 to 47293291
ttcagatcatcgattaacggtcatagatatacatcaattttattatctgatattttcggatctaaaattaatagtgacataaaaataaatgattcttacatgcactttcaaggtgatacattatatacaactagcatcacaggccacatgctgtaggaagtactcaaattaccaaagggattaaatctatctgttgctagaccaagctggatatttagcgggtcactcgcaaagtgtggc

SEQ ID NO: 53 - DNA, 240 bp, Elaeis guineensis
  misc_feature (1)..(240): Elaeis guineensis linkage group LG5 48857594 to 48857833
ccctcaacaatctttacaaatctgctcccataaatagatgaataggtcgggactaatcaacaaagtaacaacaagaagtggggcaaatttaatgtttaaatctgcagataaacagaaaaattgaagagaagtttgatgatatttatgcatcatggttcaaggtccggccaaatctatttcgcagtattaaatctcattacagataaagcggcagaaagagaagctatcaagcacttgata

SEQ ID NO: 54 - DNA, 240 bp, Elaeis guineensis
  misc_feature (1)..(240): Elaeis guineensis linkage group LG5 48932047 to 48932286
agatcatttagaaaatgattcctagagtggtcctagtggaggtcttgaaggcatacaagcagtggtacaatctgttcttatgaagaatgattagcagtggacttgcaaagatgaatgtagtgctacatcaaattaggatggattttgatcagtctcatgtggatcatcattggagggcttctactttggagtcttgtggagatctcatgattaccagatccttgtcttcttgttcgtatg

SEQ ID NO: 55 - DNA, 240 bp, Elaeis guineensis
  misc_feature (1)..(240): Elaeis guineensis linkage group LG6 5943980 to 5944219
atcggtttcagtattgctagactccttgttgctaaggtcccagaaatatctaaatccacacatgggactatccgatccttgaggtttagaagatcagtgtccagatgaggagccagctttggtggtggtataaggggtatcctcgaacctagcctcgaaaagaggcagtcaaagactttggccaccttgacgactttcacccctaaccttgtcaccatcaaaagtcatggaaaagaaaga

SEQ ID NO: 56 - DNA, 240 bp, Elaeis guineensis
  misc_feature (1)..(240): Elaeis guineensis linkage group LG6 6002478 to 6002717
gcatgtccgaatagcaacctactagcacaaagttattcgatactacaacttcagagtttggaacaagattttcaaggtcagcgacctagtcctatgcaggacagaagtttcttaacctagaaaatgagacaagctaaccccaaactgggagggcccataccagatagatgaagtcatcaaatcagaaatctacaggctgaaacaactagattgaatcccactctcctgaccataaaattt

SEQ ID NO: 57 - DNA, 240 bp, Elaeis guineensis
  misc_feature (1)..(240): Elaeis guineensis linkage group LG6 6337822 to 6338061
tattatatttaaaaagaactttatgaattaaaattaatgatctaattagtttagtatgataatattctgattgtaagctttagggaattataattaatgcattaagcataaaaaactttgaagaaatgtgtcagtttatactcctttggtggttccttaagacaggcatgttcccaccgcactgttctttgtgaatttatatgtttggaagcttcataaagttaaataggggagttaaag

SEQ ID NO: 58 - DNA, 240 bp, Elaeis guineensis
  misc_feature (1)..(240): Elaeis guineensis linkage group LG6 6562993 to 6563232
atttagaagcatccttgcacttctgcatgaacatcttctttgctgatgctttgccccatgttgtcgactccttcaaatgctatggtactgtaagagaggcaagctatcttaagttttaggccactatatccttcccaaatattattctgcttcagagcaatctttgcatgtatcacttcccaaggttaccatagaaagtaattttttaagtataggtactaatcttgaaggtcatccctg

SEQ ID NO: 59 - DNA, 240 bp, Elaeis guineensis
  misc_feature (1)..(240): Elaeis guineensis linkage group LG6 6818733 to 6818972
aaggcaatatcattattctttaggaggtcgactgtcagagtctctgtactggcatgccttttgattgatggaatgataattttaagtacaaactgttccttttttttttagccttttgttgttttgtgcacgactctagtaaaataaggcttctggaaccagggattaatttctgaatttctgatggaaaaacaggttggatttataaattcacttgcatttgcaaaatctggacgtttc

SEQ ID NO: 60 - DNA, 240 bp, Elaeis guineensis
  misc_feature (1)..(240): Elaeis guineensis linkage group LG6 7281419 to 7281658
cttggtgcaatggtagctgtatgggcaggtagatgcttgaagttgctggtaagtgctttttggtgttggcttcgtttgttgatcaactgcaattaaaagtttgagaaaatgaaaggaagctgttggttgctttccatctcacgtggaatcatggatatcatgcaatgcaagctcaatcgtatatgatgtaaatgctggtatatttgttctttttgcagtaaattatttgtttatttacac

SEQ ID NO: 61 - DNA, 240 bp, Elaeis guineensis
  misc_feature (1)..(240): Elaeis guineensis linkage group LG6 17578027 to 17578266
caattctttatttttcaaactaatctttttaaattcaatcataaattttaaaaatatatcttataattcatcaaatataaaatctaaagtattttcagaacttacctcatctatatgagccatcaaacataagttagccacctcttgtgtttcctctcagagcttgaatcttcattgtcgctccatgtagctatcatttccttctacttcatcttcttgactaattttttaggaaagggg

SEQ ID NO: 62 - DNA, 240 bp, Elaeis guineensis
  misc_feature (1)..(240): Elaeis guineensis linkage group LG6 18209618 to 18209857
aatggaagctcatcagtgataataccaaacatggatcaatgtcatacccaacacggaagtagtcttataatatcataccaattatgatattggaacttctattgtgcagcccaaatactcagacctttacaacgcaagggcggtagcaagggctgtcaaatgaaccaatcgtcgagctcagctcgagaaaagctcatatcaagctcaatttgaagctaaacaaatcaagtttaaaccaga

SEQ ID NO: 63 - DNA, 240 bp, Elaeis guineensis
  misc_feature (1)..(240): Elaeis guineensis linkage group LG6 26204516 to 26204755
cgttccactcctctccagagccctaaacttaatagactcaaacctacatcacaatctgaaacgatattgttcgcatcaagattttcccttgataccaagatactgtcctccgtaaattggtttcacttcatgttctgtgagatgcatctcccttgtccttatctgtggccctgctagagactttgaagcagcagcaggcaagctccggacgttgtttgaaatagacctcagcatattttt

SEQ ID NO: 64 - DNA, 240 bp, Elaeis guineensis
  misc_feature (1)..(240): Elaeis guineensis linkage group LG6 26754768 to 26755007
cttatttttttctccttatatgtgtgatattttgaggagattttttgtaaaaaattagttggagaactgatttactgtgaaaaaaatatgggaagaggcatcccatatttttttggatgtgttttgggtgtgaaaattatgaggaggcaatatctcttgaagagtccaagatttctaggactcttctcctctacccttacatgccataataaaagggttttatttgtatgtgaatgtaag

SEQ ID NO: 65 - DNA, 240 bp, Elaeis guineensis
  misc_feature (1)..(240): Elaeis guineensis linkage group LG6 36492757 to 36492996
ctgccatccccacttccgcatccacatattctcacatgaaggaattgacccagctaatacaattcggcctaatgatctcacacaataaaactagcttcaactatgttagcatgaactgatgtatcgaggttcaatttcacttgcccggggctggggaactccggacgccatggcctagcgaagaacttgctgttgttctggttagaatacttcttagaataaggccactatcacttcaat

SEQ ID NO: 66 - DNA, 240 bp, Elaeis guineensis
  misc_feature (1)..(240): Elaeis guineensis linkage group LG6 36494518 to 36494757
ttgaattgtagtttttcctatacttatatggggtttcccaaagaaagattggttcatagctgtaggttggctccgaagattccaccaaatatagcagagaagggcttaatcttgttagttccagcgaacattccagctttcaacatgctttccaagcaaattcgacttgaagttggggtaatattttgtgcaggcctactcttagtcgagtgaaaataatcaacgtgcagtgattaattg

SEQ ID NO: 67 - DNA, 240 bp, Elaeis guineensis
  misc_feature (1)..(240): Elaeis guineensis linkage group LG7 219790 to 220029
atggtttggtctaaaggtataaatgggtcagatcagatcgaatcatgaattattttgattcaatccgattttttgatcgagtcttgatattggacttagattcagcccaataaaagattggatcggataggcttggatctataatcaaatttcttgatccaatggattattcagatcgaatcggatccatgtataatccgatcttaattcataaaatataaatccgattcggatctagat

SEQ ID NO: 68 - DNA, 240 bp, Elaeis guineensis
  misc_feature (1)..(240): Elaeis guineensis linkage group LG7 1532910 to 1533149
atcagggggaaagatacagttttagaatcaagttcagctggataggggtatgaaggaagatacagttctgctggatggatatagctactgatgtatttatgaagaagaacgaataacaagagatagcataaaagggttttaggtgcaaatggctcaatatatagatgctataggaaaggtgctacattggcaacataatcaaagtgcaaattcaagtatttcaggctacagcaaacacca

SEQ ID NO: 69 - DNA, 240 bp, Elaeis guineensis
  misc_feature (1)..(240): Elaeis guineensis linkage group LG8 8700733 to 8700972
gaaaatagggtaggggttgtatcatgcaaaagaataattgatcttctttcggagtgtgaaccattggatcatactttgtacaactttcaaaatgctccaacctcttctttctatttatcagcatatatcatgaaatagacatcgtgctctgaaagctgtggaaagtgcgatcaaatgattcactccatgaaggaaggtcaatcattctttcatgtgaaagacacaatctgaaccacatat

SEQ ID NO: 70 - DNA, 240 bp, Elaeis guineensis
  misc_feature (1)..(240): Elaeis guineensis linkage group LG8 9242093 to 9242332
aattatgttagcttccagagcaaggtagtattttaattttttcagaaaaacccctttattatttttaccttcataagtgttgaatgatgctaaaaataattgattcatactatcaagggcaaattccattcatggagcttttgccttaatcttggtcttaaaatagttttggtaacaatcaaggatttcatctaaaaaggccagctagtggatattattttagttccttgatcttgtatg

SEQ ID NO: 71 - DNA, 240 bp, Elaeis guineensis
  misc_feature (1)..(240): Elaeis guineensis linkage group LG8 23767318 to 23767557
tgtctcccagatgtaaagaaaagcacaactagtcctggaccaacctgatatgtatgatgcaaaaaatggatcaggtacatggtacgttaaagaaaaatattggacgaatactattattcggatctttgtcttcagacaatcgaacttctaccaagttaatttgatcattttaatgtatattgatattattgatgctcgagtaatcgttgctcttgcttggaaggaaaagaagacaaaata

SEQ ID NO: 72 - DNA, 240 bp, Elaeis guineensis
  misc_feature (1)..(240): Elaeis guineensis linkage group LG8 23957413 to 23957652
aaaaattatttaaaaaaattgaattaatttaaaaattagaaatattatttatccagtaatttttttggatcaatctgatatttttttaaagcagaactggacttttataccctcattgtgtttgtacagcgatctttcgctggaactggaccggccaatggcccggtttgatgaatttctagcccggccaggcaatccggtccttgatttcccgttagggttagggtttgttcaaacata

SEQ ID NO: 73 - DNA, 240 bp, Elaeis guineensis
  misc_feature (1)..(240): Elaeis guineensis linkage group LG8 26648547 to 26648786
ggcctgttttaatggccatgaaaaactttagcgaaattaaaaacggggacagtgcatatgaaacaggaacggctacgtatatatacaccggggactgaaatgaaatattgttagaccccaatcattatatggcgatccaacgaagggccataattatttcttcccatgtttttaacaaggccttcccaaggatcgcctgggactttttgctcttgaaggttgggaagtttatatattaaa

SEQ ID NO: 74 - DNA, 240 bp, Elaeis guineensis
  misc_feature (1)..(240): Elaeis guineensis linkage group LG8 26847863 to 26848102
gatctattgtatgtgggtgacaagttacaatgcgaatctggtgtgagtgggaatgcaggatgtatgtgaagtctactaccgcaatctgcttgtaactggagtgtaaagctaagagcaacaacattcagtgtgtacttgtcacatcatcctatcatgcaagaccaagacagggatatagggttatttttcctttcttctgttctctatacaaaaacttggaattatgctatgtctataggg

SEQ ID NO: 75 - DNA, 240 bp, Elaeis guineensis
  misc_feature (1)..(240): Elaeis guineensis linkage group LG9 606020 to 606259
tgctcttggccaaacttggtaccaaggaaaacactacataattgaggcctgccatgggacaaaagctaattgttgtcattgaactttaaaatgaagtaaccttccatttcttcctttctgcattatcaaacaaactgcaatcactagacaattacatgctatgtttgatatgtttatggggatttgatttcgcctctctttcataatttttctatgattttcggggtacaagggagtttt

SEQ ID NO: 76 - DNA, 240 bp, Elaeis guineensis
  misc_feature (1)..(240): Elaeis guineensis linkage group LG9 1308992 to 1309231
ttcaacccaaggtggcacagtcgtggctccctaattgaagctcactggtgatggatgatgccggacggcatcgaaaaaaagcatgaaaatggacggtaaaataagggttcccaaaatctcaattttctttcaaaatacgtcaaataaaaatccaaaatctcagtttaaatttcagaaagagatcaaaaagaaattttttaaaagattacctgggtcttcaggatctttggacctcaaaac

SEQ ID NO: 77 - DNA, 240 bp, Elaeis guineensis
  misc_feature (1)..(240): Elaeis guineensis linkage group LG9 3499347 to 3499586
tctccaattgttcataagctattagttcctcagtcaaatgtcttctcaagttctttttgaataattttattcttcaaaacccgcttaataattcgcatgtacatgtaagtcaatggtatgagttatttatttgttatttacttattctaaattttgtgagaatgaggataacatgaacaagtgataattgaaccaaatatatatatatatatatacacacacacacacacgatgaatttt

SEQ ID NO: 78 - DNA, 240 bp, Elaeis guineensis
  misc_feature (1)..(240): Elaeis guineensis linkage group LG9 3638196 to 3638435
atgatctagaatcaaaagcactgctgttattcaaagatatcatcataactccaaaccaaggggctaacactagactaaaggaactatatcattaggttgtaaactgtaaatcaaatgtacttatcaaacaagttctgatcaaaagtttagaaaaggttggaagtgagttagttagctacttgactttgaacatggttcaagaagcatttagatgatcataccacatataggtttcatctt

SEQ ID NO: 79 - DNA, 240 bp, Elaeis guineensis
  misc_feature (1)..(240): Elaeis guineensis linkage group LG9 28437588 to 28437827
cccatttctgtacagcaaagacaataaccaaataactctttttcatatactgatagcatctggtgctttagccccaaagatctacttatgaaggcaatgggatggttgtcctgcatcagcactgctcttatcccatagttgcttgcatcagtttctaccacaaatatcttgctgaaatcaagaaaagctagcactgaagctgaaaaaagtgctactttaaggtcctcaaaagcctcattt

SEQ ID NO: 80 - DNA, 240 bp, Elaeis guineensis
  misc_feature (1)..(240): Elaeis guineensis linkage group LG9 28513432 to 28513671
gacacacctatacaggtttggaaggggggttgcttcctaggggaatttaggctgtttcatagatcaaccatgagactgagataggcacaaaagccttaatgtgcaaacatgttatctgtgatctgtgttgtatattgaatttgtaattttggtccaatgctcaagacattagtcagctttcccttgcattaattgtcacatatgtatactaggtaaagattcaagttatgaagtaacgtt

SEQ ID NO: 81 - DNA, 240 bp, Elaeis guineensis
  misc_feature (1)..(240): Elaeis guineensis linkage group LG9 28581068 to 28581307
atgctcggcctgctatctttattaatgcttgaagaatatatgagattcttggtctttctattttttatgattgaatgcaggtaagtccttcaataatatgagacacacataaattcaagagtggttgattgaacaggatgggtctttggctggagaggatccaaactcaaccagacatttgctcctttaacaatttagttagattgtagtgataaacagtaacaatcacaatgatatagt

SEQ ID NO: 82 - DNA, 240 bp, Elaeis guineensis
  misc_feature (1)..(240): Elaeis guineensis linkage group LG9 28911795 to 28912034
gctgagttatctattttgggccaaaaattgggctgacacgaacattctacgtgaaattcttatttctacgaacactcaaacccctccatgcatgggaagatagagaagatgccatgaatgagagtacagaaaatgaatccaaattaccacatccgaaaagccatagcaagcggtttctcttacaagtgatttcattaagatcgatttagtgaccaaccattcacctcccaaacattgcta

SEQ ID NO: 83 - DNA, 240 bp, Elaeis guineensis
  misc_feature (1)..(240): Elaeis guineensis linkage group LG9 32327318 to 32327557
ttgcccggtcctcactgatggctatccactgttgcccgccgacgtgttgtcgtccattatcgaacctctggttgccggaccactgaccattctgccacccttattcaaccaaaggagaaaacaagaagaaaacaaggagtaagaagaagagagagaagaggacatgtctctctctctattgtgtttctctcacttcttgttctttctctctctaagtggatatagggacccaagaaaagg

SEQ ID NO: 84 - DNA, 240 bp, Elaeis guineensis
  misc_feature (1)..(240): Elaeis guineensis linkage group LG9 32434082 to 32434321
ccgcctttttcccggaaaagggaaggaatttgaagttgcggtgagaggagagagtgcgggagaggaaacttccgtgctgaccgggtcaaccgagtcaacactaaaatcaacggctatgattagtggacccagtgcgagtagatggagagagattatgggatgaatcagaattattggctgataataaaaataatctggaaatatactacatcgtgtgtcgtggctgggagagatgaacca

SEQ ID NO: 85 - DNA, 240 bp, Elaeis guineensis
  misc_feature (1)..(240): Elaeis guineensis linkage group LG9 32538074 to 32538313
gggtcatagatatgagtcatgtattttaatctattcgatattgattttgaattgatcatgtttgatgttcatgtgtatatgatttaaacaagatgtttttatgatgtgttaggtcattttttgtatcattttttatccaatcgagttatctagtatcatatctatctctgattgcagtgtaggtgaggttgctttaatgcaaagaccttcaatcactcgaagacagaaacataaagcaaa

SEQ ID NO: 86 - DNA, 240 bp, Elaeis guineensis
  misc_feature (1)..(240): Elaeis guineensis linkage group LG9 32539835 to 32540074
aagcatctgctccttccaagccttcgaaatcaaattcttcaaaaagcatgcagctatatttatcgcattactctcaattccttggagaccttaaattccagagtccagtatttatgcataatttattgatgcctaagctcatcaaataaatctctgaatcaaaattctcagatatttctacttatgcaataaaatcattcaactatcagaaagaagaagaaaaattcagaaacctcttta

SEQ ID NO: 87 - DNA, 240 bp, Elaeis guineensis
  misc_feature (1)..(240): Elaeis guineensis linkage group LG9 32775289 to 32775528
caaagaaaacttgcacaagctgattacttatatgtaactcaacttttgtgttgacccacaaagtatggaatgtgcaacttgacatttttgtaaaaagtttgatatcatattgttcttgcgtacttggctcaaaaagatactgtgcttgtgcttccaaggagggcacgcaacctttttttttttttgatcttttcgagaataggaggatttgcagcttgaaactgtttcccaagcatccta

SEQ ID NO: 88 - DNA, 240 bp, Elaeis guineensis
  misc_feature (1)..(240): Elaeis guineensis linkage group LG9 33054457 to 33054696
ctgttacataaatacaatgtgccaattgtgcgtttctcaccatatttagaatttttattttggtatattgttggctaagcattctgtgcaaaatgcagtaagatttcttccaagcagaagttcctgataagcagtgcatttcctaagtcacatgcatggtgagcctagcaattagaaggtcaagattatgtcatatttttcagggatttgcaaatgtaccttgactcttggtcaacctat

SEQ ID NO: 89 - DNA, 240 bp, Elaeis guineensis
  misc_feature (1)..(240): Elaeis guineensis linkage group LG9 33133902 to 33134141
ttatcgttagagtgtagtcacaaaagactgcctcgatccaatagccctacctatgttgatcaataaaatttgatttcaacaatcatgaacgaatgcatgatgatctggtagatagagatcactgatagatctgaaaatttacaaagatgatctcaatgatgtttgtagtatactatcaaaattttacttcaatcggacttcattatcataatcaatttagcatgaaataatttcgattgt

SEQ ID NO: 90 - DNA, 240 bp, Elaeis guineensis
  misc_feature (1)..(240): Elaeis guineensis linkage group LG9 33253868 to 33254107
tttatctatttatagcaacttcctgctttaatttggtaaggtaaaatacctttgtctcccgttgttccagaaaaagattgcacagagttgtacacataaggctttcttttagggtaaaatggtcacacacaaggctttgcagcgaactaggtatcatagcaagtgatgggtgtgatcacgaattctctctttgtttttactcaaggaaatccaagataaaaattgggatgcatttatggt

SEQ ID NO: 91 - DNA, 240 bp, Elaeis guineensis
  misc_feature (1)..(240): Elaeis guineensis linkage group LG10 15342814 to 15343053
ctttgtagatctaagctgttttggtaggagatttatctttctccttccatcttctttttcaaatttacctattccttcaagaggtgggcattaaaagaccattagaaatagtaatattctttcctttggatgcttctaaacatgtgcttttatctgtattttctttctttcttccatgttttctttctgtctttcgaagggaagctgataactgggcggaaaaaaatggaagttttattt

SEQ ID NO: 92 - DNA, 240 bp, Elaeis guineensis
  misc_feature (1)..(240): Elaeis guineensis linkage group LG10 15405714 to 15405953
ggttctgagggttgtccatcactaacattcatgaactctatgtcaatccgatctacctcatcctcggagatgtcattgtcagaaacccccttcgaaagagtagatgtgtccttgtgcctaccagtgactgtgttctttgagaaaaggatgtcaatgtcatcataggtagggaaggacttacccttaatacgagcatactccttgtttacctattatgaatacaaataaacaagtatgatc

SEQ ID NO: 93 - DNA, 240 bp, Elaeis guineensis
  misc_feature (1)..(240): Elaeis guineensis linkage group LG11 15933273 to 15933512
taatgacttattggtcaggcggtatcactcaatcgatttattttcagatcgactgagaaatatctctctttctttaaaccttaagataaattcaagattttaactgtttagaggaatatcgagtcattttcgatgatctcaaaatatatcttagctcaccgtcattgttatcaaaatcaattgaagatgaggagttatatatatatttattcatcttatctaatgttgttagctcggttc

SEQ ID NO: 94 - DNA, 240 bp, Elaeis guineensis
  misc_feature (1)..(240): Elaeis guineensis linkage group LG11 15943724 to 15943963
tctcaaaaccttcagaacaaaaaaaacttgctcccaattaggaaggaccttatcgaattatcaaagtagttcatctaggagcatatagaatcaaaaatttggatggctcaactattccatatacttggaatgctgaaaatttcaaaatgtactatcaatgagttttcaataatttttttttcaatatctcaattcttattcaaatgaagttgaatttttatgaccaaaccacagagtcac

SEQ ID NO: 95 - DNA, 240 bp, Elaeis guineensis
  misc_feature (1)..(240): Elaeis guineensis linkage group LG12 12178551 to 12178790
ctcagaaactgagttttcagctgtcgcgcaatccctgacaactccgaccagtatccagcgaacattgccggccaaaaatgggttgcaggacatgcgagacgcccaaagtgaattataggaaaggcttgtggtctcctgaagaagatcagaaactgagggattacatgcttaagcatgggcacagctgctggagtgcagttcctgtcaaggctggtgagttcctacattatagcagaattc

SEQ ID NO: 96 - DNA, 240 bp, Elaeis guineensis
  misc_feature (1)..(240): Elaeis guineensis linkage group LG12 12249454 to 12249693
atttgatgggctaggaggtttctgggtgatgtgaggagtcagaaatcgatttctacttcgatcaggactcagattcaggcatttaaaactcctgtgggcaacagagaaaagcagaccgtggtagcagcagcgtgatctgcagagaatcctatggtttggcagtagcagcctgaagagcattggcagagagcagaggtacaggggtacctcgggcagactatcagatagcgatcgttccag

SEQ ID NO: 97 - DNA, 240 bp, Elaeis guineensis
  misc_feature (1)..(240): Elaeis guineensis linkage group LG13 2052746 to 2052985
agagtcgtcaagctattagaagaattcttttaagtatttttgctcccaagaagttggccgtaattcttcttgtgcattcttttctttctcttaataaattgttgggggaaatccttcttctcccactgtcccacaataaaaataatatttgtaaaatgatattatttacatatgttactgagcataaaaattgccttctttgtaggttccaaattgggcaatgagatgggagcattcata

SEQ ID NO: 98 - DNA, 240 bp, Elaeis guineensis
  misc_feature (1)..(240): Elaeis guineensis linkage group LG13 2447483 to 2447722
ttatccagacaacatactacgaatcttatattaaaaagaattcttgatgctagagatgcttcttcaggaatatcaataactctcagcacacaaaacaatcattgccagaatgctacgactggaatccactttgacaattaggaaaaattctaatgttccatgcagtggatgtcacatcacgacagaataagcatctttgaaccttcaataatccaacaaacagagaaggtaacaaaccat

SEQ ID NO: 99 - DNA, 240 bp, Elaeis guineensis
  misc_feature (1)..(240): Elaeis guineensis linkage group LG13 14345084 to 14345323
cagattagacttatattttttgcatggaagagccattcacttatgtgggaccatccattagttgtcttgaggtttgtgcatatggatgaatgatgaactttggttggaatgatgaactataattactttgagatctctatagtggttgatctaacaagaaattatattctaaaccgcatgcatgcactatagaaaattttaatctgattgtgaaaataaatgtacctccagctattattg

SEQ ID NO: 100 - DNA, 240 bp, Elaeis guineensis
  misc_feature (1)..(240): Elaeis guineensis linkage group LG13 14709411 to 14709650
ctgctggacctcctgctgctcctccatgccatctcacctttggagaactaaattttcatcaaaggtgacatctctagtcataacaaccttcttctccaagggatcccacagtcggtatcccttaactcctcgcagatatcccaagaacaccatcttccgtgacttggcatccaatttgctttgctcaccagcaccaatgtgcacaaatgccatgcacctaaagacccttagatggctccg

SEQ ID NO: 101 - DNA, 240 bp, Elaeis guineensis
  misc_feature (1)..(240): Elaeis guineensis linkage group LG13 22031000 to 22031239
gttttgaaattaattttccatcgtctgccaccttccatttatgcaggattttggaaattatccaatgttgcagttttagaggatagaataaacttcatggttaaatttgctgtcaaaataagtgatcaccttttagcacaaataagaaagcccttaatttgtttataaatccagtacatcctcctagcctttcaaacaaatataggtagcccctacccttcaccattattgttagttcta

SEQ ID NO: 102 - DNA, 240 bp, Elaeis guineensis
  misc_feature (1)..(240): Elaeis guineensis linkage group LG13 22147321 to 22147560
taacaatgttcacccgggcagattagaagtaaagcagacctatcaaagattctggatgactataatgttggagataaagttgttctgaaggttcaaagaggtaaccggagcttggaactgcctttggtcttagaggagacaagcatttgatgagcacccaaaatcctccatctgtctaattgtgcatatccaagcttgtgaaattaataggtaattcattctttcactattttccctctt

SEQ ID NO: 103 - DNA, 240 bp, Elaeis guineensis
  misc_feature (1)..(240): Elaeis guineensis linkage group LG15 23588504 to 23588743
tctaattgctaatatggaggcacttagaaaatacaatcaggtaaagctatttttgaggaacttaaatgggccgtcaacagaacaatctttaatcaatttcattccttctgtgctcgatataggtcatgagcttcctgttaaaggttaagcgtgcaaaatttgttcttgacaaagctcggaagtggatgtggaaggtttatacccatttaatttcttcttgttatccgtttgtatcatcca

SEQ ID NO: 104 - DNA, 240 bp, Elaeis guineensis
  misc_feature (1)..(240): Elaeis guineensis linkage group LG15 24307111 to 24307350
agtactaccactcccctcttccacgtgaggcgccttttgcggggcaaaaccgtgcggggcgggtgttaaccgggtcaaaggcctgggccccgttccagagtggacaatacctcacgctgggtggaggagatgggatcggtacgactgagcagtagacctcaacacatagcttatgaccaaattcaacattttgcctaacaaacatatgcatagagtgtttaagctattagtcctaagaaa

SEQ ID NO: 105 - DNA, 240 bp, Elaeis guineensis
  misc_feature (1)..(240): Elaeis guineensis linkage group LG16 1511530 to 1511769
taaactaaggtatagagggaacattggcatattagcaagattgttagaagaacgaagaccaacttattaatagggaagtgcttaatagacctacttacctgatgcttccatgccttctcccactccatcagtgatgagtctgcttcgatcgctcggatggccacgattaatcccctgttgtcccccataagtatagttggggcattgccatttggtaagagaagccatggattttggcgg

SEQ ID NO: 106 - DNA, 240 bp, Elaeis guineensis
  misc_feature (1)..(240): Elaeis guineensis linkage group LG16 1595781 to 1596020
tcttgaagctcgagaactccccacatgaaaagaacttgcactgttgcgagaatgcttcagtactaaaaagatgaagattagcacaccaaagcagctaaccgacacggtccaatattgcatgttcttataagcaagctctgcccatgcaaagagagattattagattcagataaagtaataacataaattattcaaggcaagaaaatgaagatgaaatataaaagaagtacaagacactgc

SEQ ID NO: 107 - DNA, 240 bp, Elaeis guineensis
  misc_feature: Elaeis guineensis linkage group LG16 2684531 to 2684770
tgctgtcactttaactaaagactagacaaatgacaaatggaaaatttcttggataacttaagactgaccatggtccagttgctatcagaagaacaagaaaataaatatatatatttcagcatggagtattggtatcttacattttcagtggacatggtagcccgaaaaaaaagaaaaaaagactttgcttttttttggtgacgttcgaatcttgacatttatcaactaaagctctgcatg

SEQ ID NO: 108 - DNA, 240 bp, Elaeis guineensis
  misc_feature (1)..(240): Elaeis guineensis linkage group LG16 2803443 to 2803682
attgtgatgttcagtatggttgaagttatgtctatttcaagccaaatcttccaaatatgatcacggcttcatgaatcacagaaatttcaaggacttgccaaatttctagctttgatcgatcacagcttgtagtaccaatctaattcttatactatctaatttttcttgacatatatgtataaaactatgtttagaactcccatcatcagtcttaaaatagaaaaaagaaaaatctacgat

SEQ ID NO: 109 - DNA, 240 bp, Elaeis guineensis
  misc_feature (1)..(240): Elaeis guineensis linkage group LG16 5535711 to 5535950
aattagaagttgaagtatgaatgcatccattctacaccatgcatcaaactcgcttcacttattatgataaggcaatcaacgaggagaattactgccgcccaccagggcctcattcctaattgaagtaaattcttgctctgactccagatgaagatgatgagatcctcttgctgaactttctaagtagcatctatcaatgaaaaatagcaacttaggataatcgatggaggggctggtatt

SEQ ID NO: 110 - DNA, 240 bp, Elaeis guineensis
  misc_feature (1)..(240): Elaeis guineensis linkage group LG16 5995618 to 5995857
gcaaactcaggtataagttttgcatctccttcaagcagttaccactgggtactatttcataaatgttggcctccccaatatttttgtaatttccacatgattaaagaggtgttggttttagaccaagttttatttatggtaagagacaggttgatggctttgaaatccacttgatattataggtgaatcattacagaggctactgcttgctgctgaaaattttataaagcccctgaactt

SEQ ID NO: 111 - DNA, 240 bp, Elaeis guineensis
  misc_feature (1)..(240): Elaeis guineensis linkage group LG16 8379248 to 8379487
tcactctcatatcagtatgaaatcctttcacaactggtcccacgcgcacattattctcctgtttacctggtgcacgtttctgtgaacgttgaaccacatgcacccttggcaggttgaaaaatctgaagtgggtccctgtttaatttccgtcaagcctcgtggggttggtttaacgtatggtaggtcttaccgtgatgtggccatcaatacagaaatccaagcttcgctccttatgccaag

SEQ ID NO: 112 - DNA, 240 bp, Elaeis guineensis
  misc_feature (1)..(240): Elaeis guineensis linkage group LG16 8554612 to 8554851
gtgttatgacaaagaatgatagtcttttttgattttttttttggtttgatgattaggagagttcttgttccttttcataatagatttatcattttcaattaacatattctttcccttttaaagaaaaagatcttgttttcaagtccgcccaagttgctgtttgctagaatttttacaccaatgcatagagttatgtgagattaagggtgtgttggttctcaaacttcaatggaccatgct

SEQ ID NO: 113 - DNA, 240 bp, Elaeis guineensis
  misc_feature (1)..(240): Elaeis guineensis linkage group LG16 8883687 to 8883926
cgctcgattgatctagagatgaggagagccatttaatcatgaggggttgccaaaaggttagccccattgtcattatcttcggcctctgcttgactcatctggagaggagtgccatttgaccgtgtggggttcttagagggttggctccatcgtcatcatccttggcttccatttgactcatctggagatgaggagtatcatttggccaagaggggtttctagagggttagcccatggttg

SEQ ID NO: 114 - DNA, 240 bp, Elaeis guineensis
  misc_feature (1)..(240): Elaeis guineensis linkage group LG16 9269606 to 9269845
gttttaagtatttctttagaggactaagattatatagagatcatagagattgattttaaatctaaggggatctaaggatatggaaagaatattgaagatgtggatgatggagacataatgagggtgtgattatctatttttttaaattttctcctcaaaaggctaatattaaaggagagaaggaagggttaggattatagaagactttatttaaggatgaacaggggatgtggatggaga